SEQUENCE LISTING (53 sequences)

SEQ ID NO: 1 - DNA, 35 bp, Artificial Sequence
  Primer
ccggaattcggntggvanraytggrtnrtnkcncc

SEQ ID NO: 2 - DNA, 33 bp, Artificial Sequence
  Primer
ccggaattcrcanscrcarctntcnacnrycat

SEQ ID NO: 3 - DNA, 31 bp, Artificial Sequence
  primer
cgcggatccagagtcaaggtgacagacacac

SEQ ID NO: 4 - DNA, 33 bp, Artificial Sequence
  primer
cgcggatcctcctcatgagcacccacagcggtc

SEQ ID NO: 5 - DNA, 550 bp, Mus musculus
  CDS (59)..(436)
ttaaggtaggaaggatttcaggctctatttacataattgttctttccttttcacacagaatccctttttagaagtcaaggtgacagacacacccaagaggtcccggagagactttgggcttgactgcgatgagcactccacggaatcccggtgctgccgctaccccctcacggtcgattttgaagcctttggatgggactggattatcgcacccaaaagatataaggccaattactgctcaggagagtgtgaatttgtgtttttacaaaaatatccgcatactcatcttgtgcaccaagcaaaccccagaggctcagcaggcccttgctgcactccgacaaaaatgtctcccattaatatgctatattttaatggcaaagaacaaataatatatgggaaaattccagccatggtagtagaccgctgtgggtgctcatgagctttgcattaggttagaaacttcccaagtcatggaaggtcttcccctcaatttcgaaactgtgaattcctgcagcccgggggatccactagttctagagcggccgccacc

SEQ ID NO: 6 - protein, 126 aa, Mus musculus
NPFLEVKVTDTPKRSRRDFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 7 - DNA, 326 bp, Homo sapiens
  CDS (3)..(326)
caaaaagatccagaagggattttggtcttgactgtgatgagcactcaacagaatcacgatgctgtcgttaccctctaactgtggattttgaagcttttggatgggattggattatcgctcctaaaagatataaggccaattactgctctggagagtgtgaatttgtatttttacaaaaatatcctcatactcatctggtacaccaagcaaaccccagaggttcagcaggcccttgctgtactcccacaaagatgtctccaattaatatgctatattttaatggcaaagaacaaataatatatgggaaaattccagcgatggtagta

SEQ ID NO: 8 - protein, 108 aa, Homo sapiens
KRSRRDFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVV

SEQ ID NO: 9 - protein, 9 aa, Artificial Sequence
  amino acid encoded by oligonucleotide for PCR
GWXXWXXXP

SEQ ID NO: 10 - protein, 8 aa, Artificial Sequence
  amino acid encoded by oligonucleotide for PCR
MXVXSCXC

SEQ ID NO: 11 - DNA, 2676 bp, Mus musculus
  CDS (104)..(1231)
gtctctcggacggtacatgcactaatatttcacttggcattactcaaaagcaaaaagaagaaataagaacaagggaaaaaaaaagattgtgctgatttttaaaatgatgcaaaaactgcaaatgtatgtttatatttacctgttcatgctgattgctgctggcccagtggatctaaatgagggcagtgagagagaagaaaatgtggaaaaagaggggctgtgtaatgcatgtgcgtggagacaaaacacgaggtactccagaatagaagccataaaaattcaaatcctcagtaagctgcgcctggaaacagctcctaacatcagcaaagatgctataagacaacttctgccaagagcgcctccactccgggaactgatcgatcagtacgacgtccagagggatgacagcagtgatggctctttggaagatgacgattatcacgctaccacggaaacaatcattaccatgcctacagagtctgactttctaatgcaagcggatggcaagcccaaatgttgcttttttaaatttagctctaaaatacagtacaacaaagtagtaaaagcccaactgtggatatatctcagacccgtcaagactcctacaacagtgtttgtgcaaatcctgagactcatcaaacccatgaaagacggtacaaggtatactggaatccgatctctgaaacttgacatgagcccaggcactggtatttggcagagtattgatgtgaagacagtgttgcaaaattggctcaaacagcctgaatccaacttaggcattgaaatcaaagctttggatgagaatggccatgatcttgctgtaaccttcccaggaccaggagaagatgggctgaatccctttttagaagtcaaggtgacagacacacccaagaggtcccggagagactttgggcttgactgcgatgagcactccacggaatcccggtgctgccgctaccccctcacggtcgattttgaagcctttggatgggactggattatcgcacccaaaagatataaggccaattactgctcaggagagtgtgaatttgtgtttttacaaaaatatccgcatactcatcttgtgcaccaagcaaaccccagaggctcagcaggcccttgctgcactccgacaaaaatgtctcccattaatatgctatattttaatggcaaagaacaaataatatatgggaaaattccagccatggtagtagaccgctgtgggtgctcatgagctttgcattaggttagaaacttcccaagtcatggaaggtcttcccctcaatttcgaaactgtgaattcaagcaccacaggctgtaggccttgagtatgctctagtaacgtaagcacaagctacagtgtatgaactaaaagagagaatagatgcaatggttggcattcaaccaccaaaataaaccatactataggatgttgtatgatttccagagtttttgaaatagatggagatcaaattacatttatgtccatatatgtatattacaactacaatctaggcaaggaagtgagagcacatcttgtggtctgctgagttaggagggtatgattaaaaggtaaagtcttatttcctaacagtttcacttaatatttacagaagaatctatatgtagcctttgtaaagtgtaggattgttatcatttaaaaacatcatgtacacttatatttgtattgtatacttggtaagataaaattccacaaagtaggaatggggcctcacatacacattgccattcctattataattggacaatccaccacggtgctaatgcagtgctgaatggctcctactggacctctcgatagaacactctacaaagtacgagtctctctctcccttccaggtgcatctccacacacacagcactaagtgttcaatgcattttctttaaggaaagaagaatctttttttctagaggtcaactttcagtcaactctagcacagcgggagtgactgctgcatcttaaaaggcagccaaacagtattcattttttaatctaaatttcaaaatcactgtctgcctttatcacatggcaattttgtggtaaaataatggaaatgactggttctatcaatattgtataaaagactctgaaacaattacatttatataatatgtatacaatattgttttgtaaataagtgtctccttttatatttactttggtatatttttacactaatgaaatttcaaatcattaaagtacaaagacatgtcatgtatcacaaaaaaggtgactgcttctatttcagagtgaattagcagattcaatagtggtcttaaaactctgtatgttaagattagaaggttatattacaatcaatttatgtattttttacattatcaacttatggtttcatggtggctgtatctatgaatgtggctcccagtcaaatttcaatgccccaccattttaaaaattacaagcattactaaacataccaacatgtatctaaagaaatacaaatatggtatctcaataacagctacttttttattttataatttgacaatgaatacatttcttttatttacttcagttttataaattggaactttgtttatcaaatgtattgtactcatagctaaatgaaattatttcttacataaaaatgtgtagaaactataaattaaagtgttttcacatttttgaaaggc

SEQ ID NO: 12 - protein, 376 aa, Mus musculus
MMQKLQMYVYIYLFMLIAAGPVDLNEGSEREENVEKEGLCNACAWRQNTRYSRIEAIKIQILSKLRLETAPNISKDAIRQLLPRAPPLRELIDQYDVQRDDSSDGSLEDDDYHATTETIITMPTESDFLMQADGKPKCCFFKFSSKIQYNKVVKAQLWIYLRPVKTPTTVFVQILRLIKPMKDGTRYTGIRSLKLDMSPGTGIWQSIDVKTVLQNWLKQPESNLGIEIKALDENGHDLAVTFPGPGEDGLNPFLEVKVTDTPKRSRRDFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 13 - DNA, 2743 bp, Homo sapiens
  CDS (59)..(1183)
aagaaaagtaaaaggaagaaacaagaacaagaaaaaagattatattgattttaaaatcatgcaaaaactgcaactctgtgtttatatttacctgtttatgctgattgttgctggtccagtggatctaaatgagaacagtgagcaaaaagaaaatgtggaaaaagaggggctgtgtaatgcatgtacttggagacaaaacactaaatcttcaagaatagaagccattaagatacaaatcctcagtaaacttcgtctggaaacagctcctaacatcagcaaagatgttataagacaacttttacccaaagctcctccactccgggaactgattgatcagtatgatgtccagagggatgacagcagcgatggctctttggaagatgacgattatcacgctacaacggaaacaatcattaccatgcctacagagtctgattttctaatgcaagtggatggaaaacccaaatgttgcttctttaaatttagctctaaaatacaatacaataaagtagtaaaggcccaactatggatatatttgagacccgtcgagactcctacaacagtgtttgtgcaaatcctgagactcatcaaacctatgaaagacggtacaaggtatactggaatccgatctctgaaacttgacatgaacccaggcactggtatttggcagagcattgatgtgaagacagtgttgcaaaattggctcaaacaacctgaatccaacttaggcattgaaataaaagctttagatgagaatggtcatgatcttgctgtaaccttcccaggaccaggagaagatgggctgaatccgtttttagaggtcaaggtaacagacacaccaaaaagatccagaagggattttggtcttgactgtgatgagcactcaacagaatcacgatgctgtcgttaccctctaactgtggattttgaagcttttggatgggattggattatcgctcctaaaagatataaggccaattactgctctggagagtgtgaatttgtatttttacaaaaatatcctcatactcatctggtacaccaagcaaaccccagaggttcagcaggcccttgctgtactcccacaaagatgtctccaattaatatgctatattttaatggcaaagaacaaataatatatgggaaaattccagcgatggtagtagaccgctgtgggtgctcatgagatttatattaagcgttcataacttcctaaaacatggaaggttttcccctcaacaattttgaagctgtgaaattaagtaccacaggctataggcctagagtatgctacagtcacttaagcataagctacagtatgtaaactaaaagggggaatatatgcaatggttggcatttaaccatccaaacaaatcatacaagaaagttttatgatttccagagtttttgagctagaaggagatcaaattacatttatgttcctatatattacaacatcggcgaggaaatgaaagcgattctccttgagttctgatgaattaaaggagtatgctttaaagtctatttctttaaagttttgtttaatatttacagaaaaatccacatacagtattggtaaaatgcaggattgttatataccatcattcgaatcatccttaaacacttgaatttatattgtatggtagtatacttggtaagataaaattccacaaaaatagggatggtgcagcatatgcaatttccattcctattataattgacacagtacattaacaatccatgccaacggtgctaatacgataggctgaatgtctgaggctaccaggtttatcacataaaaaacattcagtaaaatagtaagtttctcttttcttcaggtgcattttcctacacctccaaatgaggaatggattttctttaatgtaagaagaatcatttttctagaggttggctttcaattctgtagcatacttggagaaactgcattatcttaaaaggcagtcaaatggtgtttgtttttatcaaaatgtcaaaataacatacttggagaagtatgtaattttgtctttggaaaattacaacactgcctttgcaacactgcagtttttatggtaaaataatagaaatgatcgactctatcaatattgtataaaaagactgaaacaatgcatttatataatatgtatacaatattgttttgtaaataagtgtctccttttttatttactttggtatatttttacactaaggacatttcaaattaagtactaaggcacaaagacatgtcatgcatcacagaaaagcaactacttatatttcagagcaaattagcagattaaatagtggtcttaaaactccatatgttaatgattagatggttatattacaatcattttatatttttttacatgattaacattcacttatggattcatgatggctgtataaagtgaatttgaaatttcaatggtttactgtcattgtgtttaaatctcaacgttccattattttaatacttgcaaaaacattactaagtataccaaaataattgactctattatctgaaatgaagaataaactgatgctatctcaacaataactgttacttttattttataatttgataatgaatatatttctgcatttatttacttctgttttgtaaattgggattttgttaatcaaatttattgtactatgactaaatgaaattatttcttacatctaatttgtagaaacagtataagttatattaaagtgttttcacatttttttgaaagac

SEQ ID NO: 14 - protein, 375 aa, Homo sapiens
MQKLQLCVYIYLFMLIVAGPVDLNENSEQKENVEKEGLCNACTWRQNTKSSRIEAIKIQILSKLRLETAPNISKDVIRQLLPKAPPLRELIDQYDVQRDDSSDGSLEDDDYHATTETIITMPTESDFLMQVDGKPKCCFFKFSSKIQYNKVVKAQLWIYLRPVETPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRSRRDFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 15 - DNA, 34 bp, Artificial Sequence
  oligonucleotide for PCR
cgcggatccgtggatctaaatgagaacagtgagc

SEQ ID NO: 16 - DNA, 37 bp, Artificial Sequence
  oligonucleotide for PCR
cgcgaattctcaggtaatgattgtttccgttgtagcg

SEQ ID NO: 17 - DNA, 20 bp, Artificial Sequence
  oligonucleotide for PCR
acactaaatcttcaagaata

SEQ ID NO: 18 - DNA, 1128 bp, Baboon
  CDS (1)..(1125)
atgcaaaaactgcaactctgtgtttatatttacctgtttatgctgattgttgctggtccagtggatctaaatgagaacagtgagcaaaaagaaaatgtggaaaaagaggggctgtgtaatgcatgtacttggagacaaaacactaaatcttcaagaatagaagccattaaaatacaaatcctcagtaaacttcgtctggaaacagctcctaacatcagcaaagatgctataagacaacttttacccaaagcgcctccactccgggaactgattgatcagtatgatgtccagagggatgacagcagcgatggctctttggaagatgacgattatcacgctacaacggaaacaatcattaccatgcctacagagtctgattttttaatgcaagtggatggaaaacccaaatgttgcttctttaaatttagctctaaaatacaatacaataaagtggtaaaggcccaactatggatatatttgagacccgtcgagactcctacaacagtgtttgtgcaaatcctgagactcatcaaacctatgaaagacggtacaaggtatactggaatccgatctctgaaacttgacatgaacccaggcactggtatttggcagagcattgatgtgaagacagtgttgcaaaattggctcaaacaacctgaatccaacttaggcattgaaataaaagctttagatgagaatggtcatgatcttgctgtaaccttcccaggaccaggagaagatgggctgaatccctttttagaggtcaaggtaacagacacacccaaaagatccagaagggattttggtcttgactgtgatgagcactcaacagaatcgcgatgctgtcgttaccctctaactgtggattttgaagctcttggatgggattggattatcgctcctaaaagatataaggccaattactgctctggagagtgtgaatttgtatttttacaaaaatatcctcatactcatctggtacaccaagcaaaccccagaggttcagcaggcccttgctgtactcccacaaagatgtctccaattaatatgctatattttaatggcaaagaacaaataatatatgggaaaattccagccatggtagtagaccgctgcgggtgctcatga

SEQ ID NO: 19 - protein, 375 aa, Baboon
MQKLQLCVYIYLFMLIVAGPVDLNENSEQKENVEKEGLCNACTWRQNTKSSRIEAIKIQILSKLRLETAPNISKDAIRQLLPKAPPLRELIDQYDVQRDDSSDGSLEDDDYHATTETIITMPTESDFLMQVDGKPKCCFFKFSSKIQYNKVVKAQLWIYLRPVETPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRSRRDFGLDCDEHSTESRCCRYPLTVDFEALGWDWIIAPKRYKANYCSGECEFVFLQKYPHTHLVHQANPRGSAGPCCTPTKMSPINMLYFNGKEQIIYGKIPAMVVDRCGCS

SEQ ID NO: 20 - DNA, 1128 bp, Bovine
  CDS (1)..(1125)
atgcaaaaactgcaaatctctgtttatatttacctatttatgctgattgttgctggcccagtggatctgaatgagaacagcgagcagaaggaaaatgtggaaaaagaggggctgtgtaatgcatgtttgtggagggaaaacactacatcgtcaagactagaagccataaaaatccaaatcctcagtaaacttcgcctggaaacagctcctaacatcagcaaagatgctatcagacaacttttgcccaaggctcctccactcctggaactgattgatcagttcgatgtccagagagatgccagcagtgacggctccttggaagacgatgactaccacgccaggacggaaacggtcattaccatgcccacggagtctgatcttctaacgcaagtggaaggaaaacccaaatgttgcttctttaaatttagctctaagatacaatacaataaactagtaaaggcccaactgtggatatatctgaggcctgtcaagactcctgcgacagtgtttgtgcaaatcctgagactcatcaaacccatgaaagacggtacaaggtatactggaatccgatctctgaaacttgacatgaacccaggcactggtatttggcagagcattgatgtgaagacagtgttgcagaactggctcaaacaacctgaatccaacttaggcattgaaatcaaagctttagatgagaatggccatgatcttgctgtaaccttcccagaaccaggagaagatggactgactccctttttagaagtcaaggtaacagacacaccaaaaagatctaggagagattttgggcttgattgtgatgaacactccacagaatctcgatgctgtcgttaccctctaactgtggattttgaagcttttggatgggattggattattgcacctaaaagatataaggccaattactgctctggagaatgtgaatttgtatttttgcaaaagtatcctcatacccatcttgtgcaccaagcaaaccccagaggttcagccggcccctgctgtactcctacaaagatgtctccaattaatatgctatattttaatggcgaaggacaaataatatacgggaagattccagccatggtagtagatcgctgtgggtgttcatga

SEQ ID NO: 21 - protein, 375 aa, Bovine
MQKLQISVYIYLFMLIVAGPVDLNENSEQKENVEKEGLCNACLWRENTTSSRLEAIKIQILSKLRLETAPNISKDAIRQLLPKAPPLLELIDQFDVQRDASSDGSLEDDDYHARTETVITMPTESDLLTQVEGKPKCCFFKFSSKIQYNKLVKAQLWIYLRPVKTPATVFVQILRLIKPMKDGTRYTGIRSLKLDMNPGTGIWQSIDVKTVLQNWLKQPESNLGIEIKALDENGHDLAVTFPEPGEDGLTPFLEVKVTDTPKRSRRDFGLDCDEHSTESRCCRYPLTVDFEAFGWDWIIAPKRYKANYCSGECEFVFLQKYPHTHLVHQANPRGSAGPCCTPTKMSPINMLYFNGEGQIIYGKIPAMVVDRCGCS

SEQ ID NO: 22 - DNA, 1128 bp, Gallus gallus
  CDS (1)..(1125)
atgcaaaagctggcagtctatgtttatatttacctgttcatgcagatcgcggttgatccggtggctctggatggcagtagtcagcccacagagaacgctgaaaaagacggactgtgcaatgcttgtacgtggagacagaatacaaaatcctccagaatagaagccataaaaattcaaatcctcagcaaactgcgcctggaacaagcacctaacattagcagggacgttattaagcagcttttacccaaagctcctccactgcaggaactgattgatcagtatgatgtccagagggacgacagtagcgatggctctttggaagacgatgactatcatgccacaaccgagacgattatcacaatgcctacggagtctgattttcttgtacaaatggagggaaaaccaaaatgttgcttctttaagtttagctctaaaatacaatataacaaagtagtaaaggcacaattatggatatacttgaggcaagtccaaaaacctacaacggtgtttgtgcagatcctgagactcattaagcccatgaaagacggtacaagatatactggaattcgatctttgaaacttgacatgaacccaggcactggtatctggcagagtattgatgtgaagacagtgctgcaaaattggctcaaacagcctgaatccaatttaggcatcgaaataaaagcttttgatgagactggacgagatcttgctgtcacattcccaggaccaggagaagatggattgaacccatttttagaggtcagagttacagacacaccgaaacggtcccgcagagattttggccttgactgtgatgagcactcaacggaatcccgatgttgtcgctacccgctgacagtggatttcgaagcttttggatgggactggattatagcacctaaaagatacaaagccaattactgctccggagaatgcgaatttgtgtttctacagaaatacccgcacactcacctggtacaccaagcaaatcccagaggctcagcaggcccttgctgcacacccaccaagatgtcccctataaacatgctgtatttcaatggaaaagaacaaataatatatggaaagataccagccatggttgtagatcgttgcgggtgctcatga

SEQ ID NO: 23 - protein, 375 aa, Gallus gallus
MQKLAVYVYIYLFMQIAVDPVALDGSSQPTENAEKDGLCNACTWRQNTKSSRIEAIKIQILSKLRLEQAPNISRDVIKQLLPKAPPLQELIDQYDVQRDDSSDGSLEDDDYHATTETIITMPTESDFLVQMEGKPKCCFFKFSSKIQYNKVVKAQLWIYLRQVQKPTTVFVQILRLIKPMKDGTRYTGIRSLKLDMNPGTGIWQSIDVKTVLQNWLKQPESNLGIEIKAFDETGRDLAVTFPGPGEDGLNPFLEVRVTDTPKRSRRDFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 24 - DNA, 1131 bp, Rattus norvegicus
  CDS (1)..(1128))
atgattcaaaaaccgcaaatgtatgtttatatttacctgtttgtgctgattgctgctggcccagtggatctaaatgaggacagtgagagagaggcgaatgtggaaaaagaggggctgtgtaatgcgtgtgcgtggagacaaaacacaaggtactccagaatagaagccataaaaattcaaatcctcagtaaactccgcctggaaacagcgcctaacatcagcaaagatgctataagacaacttctgcccagagcgcctccactccgggaactgatcgatcagtacgacgtccagagggatgacagcagtgacggctctttggaagatgacgattatcacgctaccacggaaacaatcattaccatgcctaccgagtctgactttctaatgcaagcggatggaaagcccaaatgttgcttttttaaatttagctctaaaatacagtacaacaaagtggtaaaggcccagctgtggatatatctgagagccgtcaagactcctacaacagtgtttgtgcaaatcctgagactcatcaaacccatgaaagacggtacaaggtataccggaatccgatctctgaaacttgacatgagcccaggcactggtatttggcagagtattgatgtgaagacagtgttgcaaaattggctcaaacagcctgaatccaacttaggcattgaaatcaaagctttggatgagaatgggcatgatcttgctgtaaccttcccaggaccaggagaagatgggctgaatccctttttagaagtcaaagtaacagacacacccaagaggtcccggagagactttgggcttgactgcgatgaacactccacggaatcgcggtgctgtcgctaccccctcacggtcgatttcgaagcctttggatgggactggattattgcacccaaaagatataaggctaattactgctctggagagtgtgaatttgtgttcttacaaaaatatccgcatactcatcttgtgcaccaagcaaaccccagaggctcggcaggcccttgctgcacgccaacaaaaatgtctcccattaatatgctatattttaatggcaaagaacaaataatatatgggaaaattccagccatggtagtagaccggtgtgggtgctcgtga

SEQ ID NO: 25 - protein, 376 aa, Rattus norvegicus
MIQKPQMYVYIYLFVLIAAGPVDLNEDSEREANVEKEGLCNACAWRQNTRYSRIEAIKIQILSKLRLETAPNISKDAIRQLLPRAPPLRELIDQYDVQRDDSSDGSLEDDDYHATTETIITMPTESDFLMQADGKPKCCFFKFSSKIQYNKVVKAQLWIYLRAVKTPTTVFVQILRLIKPMKDGTRYTGIRSLKLDMSPGTGIWQSIDVKTVLQNWLKQPESNLGIEIKALDENGHDLAVTFPGPGEDGLNPFLEVKVTDTPKRSRRDFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 26 - DNA, 1128 bp, Meleagris gallopavo
  CDS (1)..(1125)
atgcaaaagctagcagtctatgtttatatttacctgttcatgcagattttagttcatccggtggctcttgatggcagtagtcagcccacagagaacgctgaaaaagacggactgtgcaatgcttgcacgtggagacagaatactaaatcctccagaatagaagccataaaaattcaaatcctcagcaaactgcgcctggaacaagcacctaacattagcagggacgttattaaacaacttttacccaaagctcctccgctgcaggaactgattgatcagtatgacgtccagagagacgacagtagcgatggctctttggaagacgatgactatcatgccacaaccgaaacgattatcacaatgcctacggagtctgattttcttgtacaaatggagggaaaaccaaaatgttgcttctttaagtttagctctaaaatacaatataacaaagtagtaaaggcacaattatggatatacttgaggcaagtccaaaaacctacaacggtgtttgtgcagatcctgagactcattaaacccatgaaagacggtacaagatatactggaattcgatctttgaaacttgacatgaacccaggcactggtatctggcagagtattgatgtgaagacagtgttgcaaaattggctcaaacagcctgaatccaatttaggcatcgaaataaaagcttttgatgagaatggacgagatcttgctgtaacattcccaggaccaggtgaagatggactgaacccatttttagaggtcagagttacagacacaccaaaacggtcccgcagagattttggccttgactgcgacgagcactcaacggaatctcgatgttgtcgctacccgctgacagtggattttgaagcttttggatgggactggattatagcacctaaaagatacaaagccaattactgctctggagaatgtgaattcgtatttctacagaaatacccgcacactcacctggtacaccaagcaaatccaagaggctcagcaggcccttgctgcacacccaccaagatgtcccctataaacatgctgtatttcaatggaaaagaacaaataatatatggaaagataccagccatggttgtagatcgttgcgggtgctcatga

SEQ ID NO: 27 - protein, 375 aa, Meleagris gallopavo
MQKLAVYVYIYLFMQILVHPVALDGSSQPTENAEKDGLCNACTWRQNTKSSRIEAIKIQILSKLRLEQAPNISRDVIKQLLPKAPPLQELIDQYDVQRDDSSDGSLEDDDYHATTETIITMPTESDFLVQMEGKPKCCFFKFSSKIQYNKVVKAQLWIYLRQVQKPTTVFVQILRLIKPMKDGTRYTGIRSLKLDMNPGTGIWQSIDVKTVLQNWLKQPESNLGIEIKAFDENGRDLAVTFPGPGEDGLNPFLEVRVTDTPKRSRRDFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 28 - DNA, 1128 bp, Porcine
  CDS (1)..(1125)
atgcaaaaactgcaaatctatgtttatatttacctgtttatgctgattgttgctggtcccgtggatctgaatgagaacagcgagcaaaaggaaaatgtggaaaaagaggggctgtgtaatgcatgtatgtggagacaaaacactaaatcttcaagactagaagccataaaaattcaaatcctcagtaaacttcgcctggaaacagctcctaacattagcaaagatgctataagacaacttttgcccaaagctcctccactccgggaactgattgatcagtacgatgtccagagagatgacagcagtgatggctccttggaagatgatgattatcacgctacgacggaaacgatcattaccatgcctacagagtctgatcttctaatgcaagtggaaggaaaacccaaatgctgcttctttaaatttagctctaaaatacaatacaataaagtagtaaaggcccaactgtggatatatctgagacccgtcaagactcctacaacagtgtttgtgcaaatcctgagactcatcaaacccatgaaagacggtacaaggtatactggaatccgatctctgaaacttgacatgaacccaggcactggtatttggcagagcattgatgtgaagacagtgttgcaaaattggctcaaacaacctgaatccaacttaggcattgaaatcaaagctttagatgagaatggtcatgatcttgctgtaaccttcccaggaccaggagaagatgggctgaatccctttttagaagtcaaggtaacagacacaccaaaaagatccaggagagattttggactcgactgtgatgagcactcaacagaatctcgatgctgtcgttaccctctaactgtggattttgaagcttttggatgggactggattattgcacccaaaagatataaggccaattactgctctggagagtgtgaatttgtatttttacaaaaataccctcacactcatcttgtgcaccaagcaaaccccagaggttcagcaggcccctgctgtactcccacaaagatgtctccaatcaatatgctatattttaatggcaaagaacaaataatatatgggaaaattccagccatggtagtagatcgctgtgggtgctcatga

SEQ ID NO: 29 - protein, 375 aa, Porcine
MQKLQIYVYIYLFMLIVAGPVDLNENSEQKENVEKEGLCNACMWRQNTKSSRLEAIKIQILSKLRLETAPNISKDAIRQLLPKAPPLRELIDQYDVQRDDSSDGSLEDDDYHATTETIITMPTESDLLMQVEGKPKCCFFKFSSKIQYNKVVKAQLWIYLRPVKTPTTVFVQILRLIKPMKDGTRYTGIRSLKLDMNPGTGIWQSIDVKTVLQNWLKQPESNLGIEIKALDENGHDLAVTFPGPGEDGLNPFLEVKVTDTPKRSRRDFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 30 - DNA, 1128 bp, Ovine
  CDS (1)..(1125)
atgcaaaaactgcaaatctttgtttatatttacctatttatgctgcttgttgctggcccagtggatctgaatgagaacagcgagcagaaggaaaatgtggaaaaaaaggggctgtgtaatgcatgcttgtggagacaaaacaataaatcctcaagactagaagccataaaaatccaaatcctcagtaagcttcgcctggaaacagctcctaacatcagcaaagatgctataagacaacttttgcccaaggctcctccactccgggaactgattgatcagtacgatgtccagagagatgacagcagcgacggctccttggaagacgatgactaccacgttacgacggaaacggtcattaccatgcccacggagtctgatcttctagcagaagtgcaagaaaaacccaaatgttgcttctttaaatttagctctaagatacaacacaataaagtagtaaaggcccaactgtggatatatctgagacctgtcaagactcctacaacagtgtttgtgcaaatcctgagactcatcaaacccatgaaagacggtacaaggtatactggaatccgatctctgaaacttgacatgaacccaggcactggtatttggcagagcattgatgtgaagacagtgttgcaaaactggctcaaacaacctgaatccaacttaggcattgaaatcaaagctttagatgagaatggtcatgatcttgctgtaaccttcccagaaccaggagaagaaggactgaatccttttttagaagtcaaggtaacagacacaccaaaaagatctaggagagattttgggcttgattgtgatgagcactccacagaatctcgatgctgtcgttaccctctaactgtggattttgaagcttttggatgggattggattattgcacctaaaagatataaggccaattactgctctggagaatgtgaatttttatttttgcaaaagtatcctcatacccatcttgtgcaccaagcaaaccccaaaggttcagccggcccttgctgtactcctacaaagatgtctccaattaatatgctatattttaatggcaaagaacaaataatatatgggaagattccaggcatggtagtagatcgctgtgggtgctcatga

SEQ ID NO: 31 - protein, 375 aa, Ovine
MQKLQIFVYIYLFMLLVAGPVDLNENSEQKENVEKKGLCNACLWRQNNKSSRLEAIKIQILSKLRLETAPNISKDAIRQLLPKAPPLRELIDQYDVQRDDSSDGSLEDDDYHVTTETVITMPTESDLLAEVQEKPKCCFFKFSSKIQHNKVVKAQLWIYLRPVKTPTTVFVQILRLIKPMKDGTRYTGIRSLKLDMNPGTGIWQSIDVKTVLQNWLKQPESNLGIEIKALDENGHDLAVTFPEPGEEGLNPFLEVKVTDTPKRSRRDFGLDCDEHSTESRCCRYPLTVDFEAFGWDWIIAPKRYKANYCSGECEFLFLQKYPHTHLVHQANPKGSAGPCCTPTKMSPINMLYFNGKEQIIYGKIPGMVVDRCGCS

SEQ ID NO: 32 - DNA, 480 bp, Rattus norvegicus
  CDS (1)..(390)
gaagatgggctgaatccctttttagaagtcaaagtaacagacacacccaagaggtcccggagagactttgggcttgactgcgatgaacactccacggaatcgcggtgctgtcgctaccccctcacggtcgatttcgaagcctttggatgggactggattattgcacccaaaagatataaggctaattactgctctggagagtgtgaatttgtgttcttacaaaaatatccgcatactcatcttgtgcaccaagcaaaccccagaggctcggcaggcccttgctgcacgccaacaaaaatgtctcccattaatatgctatattttaatggcaaagaacaaataatatatgggaaaattccagccatggtagtagaccggtgtgggtgctcgtgagctttgcattagctttaaaatttcccaaatcgtggaaggtcttcccctcgatttcgaaactgtgaatttatgtaccacaggctgtag

SEQ ID NO: 33 - protein, 130 aa, Rattus norvegicus
EDGLNPFLEVKVTDTPKRSRRDFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 34 - DNA, 790 bp, Gallus gallus
  CDS (1)..(678)
ttagtagtaaaggcacaattatggatatacttgaggcaagtccaaaaacctacaacggtgtttgtgcagatcctgagactcattaagcccatgaaagacggtacaagatatactggaattcgatctttgaaacttgacatgaacccaggcactggtatctggcagagtattgatgtgaagacagtgctgcaaaattggctcaaacagcctgaatccaatttaggcatcgaaataaaagcttttgatgagactggacgagatcttgctgtcacattcccaggaccaggagaagatggattgaacccatttttagaggtcagagttacagacacaccgaaacggtcccgcagagattttggccttgactgtgatgagcactcaacggaatcccgatgttgtcgctacccgctgacagtggatttcgaagcttttggatgggactggattatagcacctaaaagatacaaagccaattactgctccggagaatgcgaatttgtgtttctacagaaatacccgcacactcacctggtacaccaagcaaatcccagaggctcagcaggcccttgctgcacacccaccaagatgtcccctataaacatgctgtatttcaatggaaaagaacaaataatatatggaaagataccagccatggttgtagatcgttgcgggtgctcatgaggctgtcgtgagatccaccattcgataaattgtggaagccaccaaaaaaaaaagctatatcccctcatccatctttgaaactgtgaaattacgtacgctaggcattgcc

SEQ ID NO: 35 - protein, 226 aa, Gallus gallus
LVVKAQLWIYLRQVQKPTTVFVQILRLIKPMKDGTRYTGIRSLKLDMNPGTGIWQSIDVKTVLQNWLKQPESNLGIEIKAFDETGRDLAVTFPGPGEDGLNPFLEVRVTDTPKRSRRDFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 36 - protein, 123 aa, Homo sapiens
RPRRDAEPVLGGGPGGACRARRLYVSFREVGWHRWVIAPRGFLANYCQGQCALPVALSGSGGPPALNHAVLRALMHAAAPGAADLPCCVPARLSPISVLFFDNSDNVVLRQYEDMVVDECGCR

SEQ ID NO: 37 - protein, 118 aa, Homo sapiens
REKRQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 38 - protein, 118 aa, Homo sapiens
KRSPKHHSQRARKKNKNCRRHSLYVDFSDVGWNDWIVAPPGYQAFYCHGDCPFPLADHLNSTNHAIVQTLVNSVNSSIPKACCVPTELSAISMLYLDEYDKVVLKNYQEMVVEGCGCR

SEQ ID NO: 39 - protein, 119 aa, Homo sapiens
SRGSGSSDYNGSELKTACKKHELYVSFQDLGWQDWIIAPKGYAANYCDGECSFPLNAHMNATNHAIVQTLVHLMNPEYVPKPCCAPTKLNAISVLYFDDNSNVILKKYRNMVVRACGCH

SEQ ID NO: 40 - protein, 119 aa, Homo sapiens
LRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 41 - protein, 119 aa, Homo sapiens
SRMSSVGDYNTSEQKQACKKHELYVSFRDLGWQDWIIAPEGYAAFYCDGECSFPLNAHMNATNHAIVQTLVHLMFPDHVPKPCCAPTKLNAISVLYFDDSSNVILKKYRNMVVRSCGCH

SEQ ID NO: 42 - protein, 120 aa, Homo sapiens
EQTLKKARRKQWIEPRNCARRYLKVDFADIGWSEWIISPKSFDAYYCSGACQFPMPKSLKPSNHATIQSIVRAVGVVPGIPEPCCVPEKMSSLSILFFDENKNVVLKVYPNMTVESCACR

SEQ ID NO: 43 - protein, 116 aa, Homo sapiens
GPGRAQRSAGATAADGPCALRELSVDLRAERSVLIPETYQANNCQGVCGWPQSDRNPRYGNHVVLLLKMQARGAALARPPCCVPTAYAGKLLISLSEERISAHHVPNMVATECGCR

SEQ ID NO: 44 - protein, 122 aa, Homo sapiens
ALRLLQRPPEEPAAHANCHRVALNISFQELGWERWIVYPPSFIFHYCHGGCGLHIPPNLSLPVPGAPPTPAQPYSLLPGAQPCCAALPGTMRPLHVRTTSDGGYSFKYETVPNLLTQHCACI

SEQ ID NO: 45 - protein, 122 aa, Homo sapiens
HRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 46 - protein, 121 aa, Homo sapiens
HRIRKRGLECDGRTNLCCRQQFFIDFRLIGWNDWIIAPTGYYGNYCEGSCPAYLAGVPGSASSFHTAVVNQYRMRGLNPGTVNSCCIPTKLSTMSMLYFDDEYNIVKRDVPNMIVEECGCA

SEQ ID NO: 47 - protein, 115 aa, Homo sapiens
HRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 48 - protein, 115 aa, Homo sapiens
KKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 49 - protein, 115 aa, Homo sapiens
KKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCLCS

SEQ ID NO: 50 - protein, 4 aa, Artificial Sequence
  proteolytic cleavage site
RXXR

SEQ ID NO: 51 - protein, 4 aa, Artificial Sequence
  Eukaryotic-  proteolytic processing site
RSRR

SEQ ID NO: 52 - protein, 405 aa, Mus musculus
MVLAAPLLLGFLLLALELRPRGEAAEGPAAAAAAAAAAAGVGGERSSRPAPSAPPEPDGCPVCVWRQHSRELRLESIKSQILSKLRLKEAPNISREVVKQLLPKAPPLQQILDLHDFQGDALQPEDFLEEDEYHATTETVISMAQETDPAVQTDGSPLCCHFHFSPKVMFNKVLKAQLWVYLRPVPRPATVYLQILRLKPLTGEGTAGGGGGGRRHIRIRSLKIELHSRSGHWQSIDFKQVLHSWFRQPQSNWGIEINAFDPSGTDLAVTSLGPGAEGLHPFMELRVLENTKRSRRNLGLDCDEHSSESRCCRYPLTVDFEAFGWDWIIAPKRYKANYCSGQCEYMFMQKYPHTHLVQQANPRGSAGPCCTPTKMSPINMLYFNDKQQIIYGKIPGMVVDRCGCS

SEQ ID NO: 53 - protein, 407 aa, Homo sapiens
MVLAAPLLLGFLLLALELRPRGEAAEGPAAAAAAAAAAAAAGVGGERSSRPAPSVAPEPDGCPVCVWRQHSRELRLESIKSQILSKLRLKEAPNISREVVKQLLPKAPPLQQILDLHDFQGDALQPEDFLEEDEYHATTETVISMAQETDPAVQTDGSPLCCHFHFSPKVMFTKVLKAQLWVYLRPVPRPATVYLQILRLKPLTGEGTAGGGGGGRRHIRIRSLKIELHSRSGHWQSIDFKQVLHSWFRQPQSNWGIEINAFDPSGTDLAVTSLGPGAEGLHPFMELRVLENTKRSRRNLGLDCDEHSSESRCCRYPLTVDFEAFGWDWIIAPKRYKANYCSGQCEYMFMQKYPHTHLVQQANPRGSAGPCCTPTKMSPINMLYFNDKQQIIYGKIPGMVVDRCGCS

(19) United States
(12) Patent Application Publication
(10) Pub. No.: US 2004/0055027 A1
(43) Pub. Date: Mar. 18, 2004

(54) Growth differentiation factor-8

(75) Inventors: Se-Jin Lee, Baltimore, MD (US); Alexandra C. McPherron, Baltimore, MD (US)

Correspondence Address: Lisa A. Haile, J.D., Ph.D., GRAY CARY WARE & FREIDENRICH LLP, Suite 1100, 4365 Executive Drive, San Diego, CA, 92121-2133, US

(73) Assignee: The Johns Hopkins University School of Medicine

(21) Appl. No.: 10/463,973

(22) Filed: Jun. 17, 2003

Related U.S. Application Data
(63) Continuation of application No. 09/872,856, filed on Jun. 1, 2001.
Application No. 09/872,856 is a continuation of application No. 09/124,180, filed on Jul. 28, 1998, now abandoned.
Application No. 09/124,180 is a continuation-in-part of application No. 09/019,070, filed on Feb. 5, 1998, now abandoned.
Application No. 09/019,070 is a continuation-in-part of application No. 08/862,445, filed on May 23, 1997, now abandoned.
Application No. 08/862,445 is a continuation-in-part of application No. 08/847,910, filed on Apr. 28, 1997, now abandoned.
Application No. 08/847,910 is a continuation-in-part of application No. 08/795,071, filed on Feb. 5, 1997, now Pat. No. 5,994,618.
Application No. 08/795,071 is a continuation-in-part of application No. 08/525,596, filed on Oct. 26, 1995, now Pat. No. 5,827,733.
Application No. 08/525,596 is a 371 of international application No. PCT/US94/03019, filed on Mar. 18, 1994.
Application No. 08/525,596 is a continuation-in-part of application No. 08/033,923, filed on Mar. 19, 1993, now abandoned.

(51) Int. Cl. A01K 67/027

(52) U.S. Cl. 800/17; 800/20

(57) ABSTRACT

A transgenic non-human animal of the species selected from the group consisting of avian, bovine, ovine and porcine having a transgene which results in disrupting the production of and/or activity of growth differentiation factor-8 (GDF-8) chromosomally integrated into the germ cells of the animal is provided. Also provided are methods for making such animals, and methods of treating animals, including humans, with antibodies or antisense directed to GDF-8. The animals so treated are characterized by increased muscle tissue and bone content.

This application is a continuation of U.S. Ser. No. 09/872,856, filed Jun. 1, 2001, which is a continuation of U.S. Ser. No. 09/124,180, filed Jul. 28, 1998 (now abandoned), which is a continuation-in-part (CIP) of U.S. Ser. No. 09/019,070, filed Feb. 5, 1998 (now abandoned), which is a CIP of U.S. Ser. No. 08/862,445, filed May 23, 1997 (now abandoned), which is a CIP of U.S. Ser. No. 08/847,910, filed Apr. 28, 1997 (now abandoned), which is a CIP of U.S. Ser. No. 08/795,071, filed Feb. 5, 1997 (now U.S. Pat. No. 5,994,618), which is a CIP of U.S. Ser. No. 08/525,596, filed Oct. 25, 1995 (now U.S. Pat. No.25,827,733), which is a 371 application of PCT/US94/03019 filed Mar. 18, 1994, which is a CIP of U.S. Ser. No. 08/033,923 filed Mar. 19, 1993 (now abandoned), each of which is incorporated herein by reference.

### BACKGROUND OF THE INVENTION

1. Field of the Invention

The invention relates generally to growth factors and specifically to a new member of the transforming growth factor beta (TGF-β) superfamily, which is denoted, growth differentiation factor-8 (GDF-8) and methods of use for modulating muscle, bone, kidney and adipose cell and tissue growth.

2. Description of Related Art

The transforming growth factor β (TGF-β) superfamily encompasses a group of structurally-related proteins which affect a wide range of differentiation processes during embryonic development. The family includes, Mullerian inhibiting substance (MIS), which is required for normal male sex development (Behringer et al., Nature, 345:167, 1990), Drosophila decapentaplegic (DPP) gene product, which is required for dorsal-ventral axis formation and morphogenesis of the imaginal disks (Padgett et al., Nature, 325:81-84, 1987), the Xenopus Vg-1 gene product, which localizes to the vegetal pole of eggs (Weeks et al., Cell, 51:861-867, 1987), the activins (Mason, et al., Biochem. Biophys. Res. Comm., 135:957-964, 1986), which can induce the formation of mesoderm and anterior structures in Xenopus embryos (Thomsen et al., Cell, 63:485, 1990), and the bone morphogenetic proteins (BMPs, osteogenin, OP-1) which can induce de novo cartilage and bone formation (Sampath, et al., J. Biol. Chem., 265:13198, 1990). The TGF-βs can influence a variety of differentiation processes, including adipogenesis, myogenesis, chondrogenesis, hematopoiesis, and epithelial cell differentiation (for review, see Massague, Cell 49:437, 1987).

The proteins of the TGF-β family are initially synthesized as a large precursor protein which subsequently undergoes proteolytic cleavage at a cluster of basic residues approximately 110-140 amino acids from the C-terminus. The C-terminal regions, or mature regions, of the proteins are all structurally related and the different family members can be classified into distinct subgroups based on the extent of their homology. Although the homologies within particular subgroups range from 70% to 90% amino acid sequence identity, the homologies between subgroups are significantly lower, generally ranging from only 20% to 50%. In each case, the active species appears to be a disulfide-linked dimer of C-terminal fragments. Studies have shown that when the pro-region of a member of the TGF-β family is coexpressed with a mature region of another member of the TGF-β family, intracellular dimerization and secretion of biologically active homodimers occur (Gray et al., Science, 247:1328, 1990). Additional studies by Hammonds et al., (Molec. Endocrinol. 5:149, 1991) showed that the use of the BMP-2 pro-region combined with the BMP-4 mature region led to dramatically improved expression of mature BMP-4. For most of the family members that have been studied, the homodimeric species has been found to be biologically active, but for other family members, like the inhibins (Ling et al., Nature, 321 :779, 1986) and the TGF-βs (Cheifetz et al., Cell, 48:409, 1987), heterodimers have also been detected, and these appear to have different biological properties than the respective homodimers.

In addition it is desirable to produce livestock and game animals, such as cows, sheep, pigs, chicken and turkey, fish which are relatively high in musculature and protein, and low in fat content. Many drug and diet regimens exist which may help increase muscle and protein content and lower undesirably high fat and/or cholesterol levels, but such treatment is generally administered after the fact, and is begun only after significant damage has occurred to the vasculature. Accordingly, it would be desirable to produce animals which are genetically predisposed to having higher muscle and/or bone content, without any ancillary increase in fat levels.

The food industry has put much effort into increasing the amount of muscle and protein in foodstuffs. This quest is relatively simple in the manufacture of synthetic foodstuffs, but has been met with limited success in the preparation of animal foodstuffs. Attempts have been made, for example, to lower cholesterol levels in beef and poultry products by including cholesterol-lowering drugs in animal feed (see, e.g. Elkin and Rogler, J. Agric. Food Chem. 38:1635-1641, 1990). However, there remains a need for more effective methods of increasing muscle and reducing fat and cholesterol levels in animal food products.

### SUMMARY OF THE INVENTION

The present invention provides a cell growth and differentiation factor, GDF-8, a polynucleotide sequence which encodes the factor, and antibodies which are immunoreactive with the factor. This factor appears to relate to various cell proliferative disorders, especially those involving muscle, nerve, bone, kidney and adipose tissue.

In one embodiment, the invention provides a method for detecting a cell proliferative disorder of muscle, nerve, bone, kidney or fat origin and which is associated with GDF-8. In another embodiment, the invention provides a method for treating a cell proliferative disorder by suppressing or enhancing GDF-8 activity.

In another embodiment, the subject invention provides non-human transgenic animals which are useful as a source of food products with high muscle, bone and protein content, and reduced fat and cholesterol content. The animals have been altered chromosomally in their germ cells and somatic cells so that the production of GDF-8 is produced in reduced amounts, or is completely disrupted, resulting in animals with decreased levels of GDF-8 in their system and higher than normal levels of muscle tissue and bone tissue, such as ribs, preferably without increased fat and/or cholesterol levels. Accordingly, the present invention also includes food products provided by the animals. Such food products have increased nutritional value because of the increase in muscle tissue and bone content. The transgenic non-human animals of the invention include bovine, porcine, ovine and avian animals, for example.

The subject invention also provides a method of producing animal food products having increased bone content. The method includes modifying the genetic makeup of the germ cells of a pronuclear embryo of the animal, implanting the embryo into the oviduct of a pseudopregnant female thereby allowing the embryo to mature to full term progeny, testing the progeny for presence of the transgene to identify transgene-positive progeny, cross-breeding transgene-positive progeny to obtain further transgene-positive progeny and processing the progeny to obtain foodstuff. The modification of the germ cell comprises altering the genetic composition so as to disrupt or reduce the expression of the naturally occurring gene encoding for production of GDF-8 protein. In a particular embodiment, the transgene comprises antisense polynucleotide sequences to the GDF-8 protein. Alternatively, the transgene may comprise a non-functional sequence which replaces or intervenes in the native GDF-8 gene.

The subject invention also provides a method of producing avian food products having improved muscle and/or bone content. The method includes modifying the genetic makeup of the germ cells of a pronuclear embryo of the avian animal, implanting the embryo into the oviduct of a pseudopregnant female into an embryo of a chicken, culturing the embryo under conditions whereby progeny are hatched, testing the progeny for presence of the genetic alteration to identify transgene-positive progeny, cross-breeding transgene-positive progeny and processing the progeny to obtain foodstuff.

The invention also provides a method for treating a muscle, bone, kidney or adipose tissue disorder in a subject. The method includes administering a therapeutically effective amount of a GDF-8 agent to the subject, thereby inhibiting abnormal growth of muscle, bone or adipose tissue. The GDF-8 agent may include an antibody, a GDF-8 antisense molecule or a dominant negative polypeptide, for example. In one aspect, a method for inhibiting the growth regulating actions of GDF-8 by contacting an anti-GDF-8 monoclonal antibody, a GDF-8 antisense molecule or a dominant negative polypeptide (or polynucleotide encoding a dominant negative polypeptide) with fetal or adult muscle cells, bone cells or progenitor cells is included. These agents can be administered to a patient suffering from a disorder such as muscle wasting disease, neuromuscular disorder, muscle atrophy, osteoporosis, bone degenerative diseases, obesity or other adipocyte cell disorders, and aging, for example. In another aspect of the invention, the agent may be an agonist of GDF-8 activity. In this embodiment, the agonist may be administered to promote kidney cell growth and differentiation in kidney tissue.

The invention also provides a method for identifying a compound that affects GDF-8 activity or gene expression including incubating the compound with GDF-8 polypeptide, or with a recombinant cell expressing GDF-8 under conditions sufficient to allow the compounds to interact and determining the effect of the compound on GDF-8 activity or expression.

### BRIEF DESCRIPTION OF THE DRAWINGS

FIG. 1A is a northern blot showing expression of GDF-8 mRNA in adult tissues. The probe was a partial murine GDF-8 clone.

FIG. 1B is a Southern blot showing GDF-8 genomic sequences identified in mouse, rat, human, monkey, rabbit, cow, pig, dog and chicken.

FIGS. 2A to 2D show partial nucleotide and predicted amino acid sequences of murine GDF-8 (FIG. 2A; SEQ ID NOS: 5 and 6, respectively), human GDF-8 (FIG. 2B; SEQ ID NOS: 7 and 8, respectively), rat GDF-8 (FIG. 2C; SEQ ID NOS: 24 and 25, respectively) and chicken GDF-8 (FIG. 2D; SEQ ID NOS: 22 and 23 respectively). The putative dibasic processing sites in the murine sequence are boxed.

FIG. 3A shows the alignment of the C-terminal sequences of GDF-8 (SEQ ID NO: 14) with other members of the TGF-β superfamily SEQ ID NOS: 36 to 49, respectively. The conserved cysteine residues are boxed. Dashes denote gaps introduced in order to maximize alignment.

FIG. 3B shows the alignment of the C-terminal sequences of GDF-8 from human (SEQ ID NO: 14), murine (SEQ ID NO: 12), rat (SEQ ID NO: 25), and chicken (SEQ ID NO: 23) sequences.

FIG. 4 shows amino acid homologies among different members of the TGF superfamily. Numbers represent percent amino acid identities between each pair calculated from the first conserved cysteine to the C-terminus. Boxes represent homologies among highly-related members within particular subgroups.

FIGS. 5A to 5D show sequences of GDF-8. Nucleotide and amino acid sequences of murine (FIGS. 5A and 5B; GenBank accession number U84005; SEQ ID NOS: 11 and 12, respectively) and human (FIGS. 5C and 5D; SEQ ID NOS: 13 and 14, respectively) GDF-8 cDNA clones are shown. Numbers indicate nucleotide position relative to the 5′ end. Consensus N-linked glycosylation signals are shaded. The putative RXXR (SEQ ID NO: 50) proteolytic cleavage sites are boxed.

FIGS. 6A and 6B show hydropathicity profiles of GDF-8. Average hydrophobicity values for murine (FIG. 6A) and human (FIG. 6B) GDF-8 were calculated using the method of Kyte and Doolittle (J. Mol. Biol., 157:105-132, 1982). Positive numbers indicate increasing hydrophobicity.

FIG. 7 shows a comparison of murine (SEQ ID NO: 12) and human (SEQ ID NO: 14) GDF-8 amino acid sequences. The predicted murine sequence is shown in the top lines and the predicted human sequence is shown in the bottom lines. Numbers indicate amino acid position relative to the N-terminus. Identities between the two sequences are denoted by a vertical line.

FIG. 8 shows the expression of GDF-8 in bacteria. BL21 (DE3) (pLysS) cells carrying a pRSET/GDF-8 expression plasmid were induced with isopropylthio-β-galactoside, and the GDF-8 fusion protein was purified by metal chelate chromatography. Lanes: total=total cell lysate; soluble=soluble protein fraction; insoluble=insoluble protein fraction (resuspended in 10 mM Tris pH 8.0, 50 mM sodium phosphate, 8 M urea, and 10 mM β-mercaptoethanol {buffer B}) loaded onto the column, pellet=insoluble protein fraction discarded before loading the column; flowthrough=proteins not bound by the column; washes=washes carried out in buffer B at the indicated pH's. Positions of molecular weight standards are shown at the right. Arrow indicates the position of the GDF-8 fusion protein.

FIG. 9 shows the expression of GDF-8 in mammalian cells. Chinese hamster ovary cells were transfected with pMSXND/GDF-8 expression plasmids and selected in G418. Conditioned media from G418-resistant cells (prepared from cells transfected with constructs in which GDF-8 was cloned in either the antisense or sense orientation) were concentrated, electrophoresed under reducing conditions, blotted, and probed with anti-GDF-8 antibodies and (¹²⁵I)-iodoprotein-A. Arrow indicates the position of the processed GDF-8 protein.

FIGS. 10A and 10B show the expression of GDF-8 mRNA. PolyA-selected RNA (5 μg each) prepared from adult tissues (FIG. 10A) or placentas end embryos (FIG. 10B) at the indicated days of gestation was electrophoresed on formaldehyde gels, blotted, and probed with full length murine GDF-8.

FIG. 11 shows chromosomal mapping of human GDF-8. DNA samples prepared from human/rodent somatic cell hybrid lines were subjected to PCR, electrophoresed on agarose gels, blotted, and probed. The human chromosome contained in each of the hybrid cell lines is identified at the top of each of the first 24 lanes (1-22, X, and Y). In the lanes designated M, CHO, and H, the starting DNA template was total genomic DNA from mouse, hamster, and human sources, respectively. In the lane marked B1, no template DNA was used. Numbers at left indicate the mobilities of DNA standards.

FIG. 12A shows a map of the GDF-8 locus (top line) and targeting construct (second line). The black and stippled boxes represent coding sequences for the pro- and C-terminal regions, respectively. The white boxes represent 5′ and 3′ untranslated sequences. A probe derived from the region downstream of the 3′ homology fragment and upstream of the most distal Hindll site shown hybridizes to an 11.2 kb HindIII fragment in the GDF-8 gene and a 10.4 kb fragment in an homologously targeted gene. Abbreviations: H, HindIII; X, Xba I.

FIG. 12B shows a Southern blot analysis of offspring derived from a mating of heterozygous mutant mice. The lanes are as follows: DNA prepared from wild type 129 SV/J mice (lane 1), targeted embryonic stem cells (lane 2), F1 heterozygous mice (lanes 3 and 4), and offspring derived from a mating of these mice (lanes 5-13).

FIGS. 13A and 13B show the muscle fiber size distribution in mutant and wild type littermates. FIG. 13A shows the smallest cross-sectional fiber widths measured for wild type (n=1761) and mutant (n=1052) tibialis cranial. FIG. 13B shows wild type (n=900) and mutant (n=900) gastrocnemius muscles, and fiber sizes were plotted as a percent of total fiber number. Standard deviations were 9 and 10 μm, respectively, for wild type and mutant tibialis cranial is and 11 and 9 μm, respectively, for wild type and mutant gastrocnemius muscles. Legend: o-o, wild type; _-_, mutant.

FIG. 14a shows the nucleotide and deduced amino acid sequence for baboon GDF-8 (SEQ ID NO: 18 and 19, respectively).

FIG. 14B shows the nucleotide and deduced amino acid sequence for bovine GDF-8 (SEQ ID NO: 20 and 21, respectively).

FIG. 14C shows the nucleotide and deduced amino acid sequence for chicken GDF-8 (SEQ ID NO: 22 and 23, respectively).

FIG. 14D shows the nucleotide and deduced amino acid sequence for rat GDF-8 (SEQ ID NO: 24 and 25, respectively).

FIG. 14E shows the nucleotide and deduced amino acid sequence for turkey GDF-8 (SEQ ID NO: 26 and 27, respectively).

FIG. 14F shows the nucleotide and deduced amino acid sequence for porcine GDF-8 (SEQ ID NO: 28 and 29, respectively).

FIG. 14G shows the nucleotide and deduced amino acid sequence for ovine GDF-8 (SEQ ID NO: 30 and 31, respectively).

FIGS. 15A and 15B show an alignment between murine, rat, human, porcine, ovine, baboon, bovine, chicken, and turkey GDF-8 amino acid sequences (SEQ ID NOS: 12, 25, 14, 29, 31, 19, 21, 23 and 27, respectively).

FIG. 16 shows the predicted amino acid sequences of murine (SEQ ID NO: 52) and human (SEQ ID NO: 53) GDF-11 aligned with murine (McPherron et al., 1997; SEQ ID NO: 12) and human (McPherron and Lee, 1997; SEQ ID NO: 14) myostatin (MSTN). Shaded boxes represent amino acid homology with the murine and human GDF-11 sequences. Amino acids are numbered relative to the human GDF-11 sequence. The predicted proteolytic processing sites are located at amino acids 295-298.

FIGS. 17A and 17B show the construction of GDF-11 null mice by homologous targeting. FIG. 17A is a map of the GDF-11 locus (top line) and targeting construct (second line). The black and stippled boxes represent coding sequences for the pro-and C-terminal regions, respectively. The targeting construct contains a total of 11 kb of homology with the GDF-11 gene. A probe derived from the region upstream of the 3′ homology fragment and downstream of the first EcoRI site shown hybridizes to a 6.5 kb EcoRI fragment in the GDF-11 gene and a 4.8 kb fragment in a homologously targeted gene. Abbreviations: X, XbaI; E, EcoRI. FIG. 17B shows a genomic Southern blot of DNA prepared from F1 heterozygous mutant mice (lanes 1 and 2) and offspring derived from a mating of these mice (lanes 3-12).

FIG. 18 shows kidney abnormalities in GDF-11 knockout mice. Kidneys of newborn animals were examined and classified according to the number of normal sized or small kidneys as shown at the top. Numbers in the table indicate number of animals falling into each classification according to genotype.

FIGS. 19A to 19J. show homeotic transformations in GDF-11 mutant mice. FIG. 19A shows newborn pups with missing (first and second from left) and normal looking tails. FIGS. 19B to 19J show skeleton preparations for newborn wild-type (FIGS. 19B, 19E and 19H), heterozygous (FIGS. 19C, 19F and 19I) and homozygous (FIGS. 19D, 19G and 19J) mutant mice. Whole skeleton preparations (FIGS. 19B to 19D), vertebral columns (FIGS. 19E to 19G), vertebrosternal ribs (FIGS. 19H to 19J) showing transformations and defects in homozygous and heterozygous mutant mice. Numbers indicate thoracic segments.

FIG. 20 is a table summarizing the anterior transforrnations in wild-type, heterozygous and homozygous GDF-11 mice.

### DETAILED DESCRIPTION OF THE INVENTION

The present invention provides a growth and differentiation factor, GDF-8 and a polynucleotide sequence encoding GDF-8. GDF-8 is expressed at highest levels in muscle and at lower levels in adipose tissue.

The animals contemplated for use in the practice of the subject invention are those animals generally regarded as useful for the processing of food stuffs, i.e. avian such as meat bred and egg laying chicken and turkey, ovine such as lamb, bovine such as beef cattle and milk cows, piscine and porcine. For purposes of the subject invention, these animals are referred to as “transgenic” when such animal has had a heterologous DNA sequence, or one or more additional DNA sequences normally endogenous to the animal (collectively referred to herein as “transgenes”) chromosomally integrated into the germ cells of the animal. The transgenic animal (including its progeny) will also have the transgene integrated into the chromosomes of somatic cells.

The TGF-β superfamily consists of multifunctional polypeptides that control proliferation, differentiation, and other functions in many cell types. Many of the peptides have regulatory, both positive and negative, effects on other peptide growth factors. The structural homology between the GDF-8 protein of this invention and the members of the TGF-β family, indicates that GDF-8 is a new member of the family of growth and differentiation factors. Based on the known activities of many of the other members, it can be expected that GDF-8 will also possess biological activities that will make it useful as a diagnostic and therapeutic reagent.

In particular, certain members of this superfamily have expression patterns or possess activities that relate to the function of the nervous system. For example, the inhibins and activins have been shown to be expressed in the brain (Meunier et al., Proc. Natl. Acad. Sci., USA, 85:247, 1988; Sawchenko et al., Nature, 334:615, 1988), and activin has been shown to be capable of functioning as a nerve cell survival molecule (Schubert et al., Nature, 344:868, 1990). Another family member, namely, GDF-1, is nervous system-specific in its expression pattern (Lee, Proc. Natl. Acad. Sci., USA, 88:4250, 1991), and certain other family members, such as Vgr-1 (Lyons et al., Proc. Natl. Acad. Sci., USA, 86:4554, 1989; Jones et al., Development, 111:531, 1991), OP-1 (Ozkaynak et al., J. Biol. Chem., 267:25220, 1992), and BMP-4 (Jones et al., Development, 111:531, 1991), are also known to be expressed in the nervous system. Because it is known that skeletal muscle produces a factor or factors that promote the survival of motor neurons (Brown, Trends Neurosci., 7:10, 1984), the expression of GDF-8 in muscle suggests that one activity of GDF-8 may be as a trophic factor for neurons. In this regard, GDF-8 may have applications in the treatment of neurodegenerative diseases, such as amyotrophic lateral sclerosis or muscular dystrophy, or in maintaining cells or tissues in culture prior to transplantation.

GDF-8 may also have applications in treating disease processes involving the musculoskeletal system, such as in musculodegenerative diseases, osteoporosis or in tissue repair due to trauma. In this regard, many other members of the TGF-β family are also important mediators of tissue repair. TGF-β has been shown to have marked effects on the formation of collagen and to cause a striking angiogenic response in the newborn mouse (Roberts et al., Proc. Natl. Acad. Sci., USA 83:4167, 1986). TGF-β has also been shown to inhibit the differentiation of myoblasts in culture (Massague et al., Proc. Natl. Acad. Sci., USA 83:8206, 1986). Moreover, because myoblast cells may be used as a vehicle for delivering genes to muscle for gene therapy, the properties of GDF-8 could be exploited for maintaining cells prior to transplantation or for enhancing the efficiency of the fusion. GDF-8 may also have applications in treating disease processes involving the kidney or in kidney repair due to trauma.

The expression of GDF-8 in adipose tissue also raises the possibility of applications for GDF-8 in the treatment of obesity or of disorders related to abnormal proliferation of adipocytes. In this regard, TGF-β has been shown to be a potent inhibitor of adipocyte differentiation in vitro (Ignotz and Massague, Proc. Natl. Acad. Sci., USA 82:8530, 1985).

Polypeptides, Polynucleotides, Vectors and Host Cells

The invention provides substantially pure GDF-8 polypeptide and isolated polynucleotides that encode GDF-8. The term “substantially pure” as used herein refers to GDF-8 which is substantially free of other proteins, lipids, carbohydrates or other materials with which it is naturally associated. One skilled in the art can purify GDF-8 using standard techniques for protein purification. The substantially pure polypeptide will yield a single major band on a non-reducing polyacrylamide gel. The purity of the GDF-8 polypeptide can also be determined by amino-terminal amino acid sequence analysis. GDF-8 polypeptide includes functional fragments of the polypeptide, as long as the activity of GDF-8 remains. Smaller peptides containing the biological activity of GDF-8 are included in the invention.

The invention provides polynucleotides encoding the GDF-8 protein. These polynucleotides include DNA, cDNA and RNA sequences which encode GDF-8. It is understood that all polynucleotides encoding all or a portion of GDF-8 are also included herein, as long as they encode a polypeptide with GDF-8 activity. Such polynucleotides include naturally occurring, synthetic, and intentionally manipulated polynucleotides. For example, GDF-8 polynucleotide may be subjected to site-directed mutagenesis. The polynucleotide sequence for GDF-8 also includes antisense sequences. The polynucleotides of the invention include sequences that are degenerate as a result of the genetic code. There are 20 natural amino acids, most of which are specified by more than one codon. Therefore, all degenerate nucleotide sequences are included in the invention as long as the amino acid sequence of GDF-8 polypeptide encoded by the nucleotide sequence is functionally unchanged.

Specifically disclosed herein is a genomic DNA sequence containing a portion of the GDF-8 gene. The sequence contains an open reading frame corresponding to the predicted C-terminal region of the GDF-8 precursor protein. The encoded polypeptide is predicted to contain two potential proteolytic processing sites (KR and RR). Cleavage of the precursor at the downstream site would generate a mature biologically active C-terminal fragment of 109 and 103 amino acids for murine and human species, respectively, with a predicted molecular weight of approximately 12,400 Da. Also disclosed are full length murine and human GDF-8 cDNA sequences. The murine pre-pro-GDF-8 protein (SEQ ID NO: 12) is 376 amino acids in length, which is encoded by a 2676 base pair nucleotide sequence (SEQ ID NO: 11), beginning at nucleotide 104 and extending to a TGA stop codon at nucleotide 1232. The human GDF-8 protein (SEQ ID NO: 14) is 375 amino acids and is encoded by a 2743 base pair sequence (SEQ ID NO: 13), with the open reading frame beginning at nucleotide 59 and extending to nucleotide 1184. GDF-8 is also capable of forming dimers, or heterodimers, with an expected molecular weight of approximately 23-30 kDa (see Example 4). For example, GDF-8 may form heterodimers with other family members, such as GDF-11.

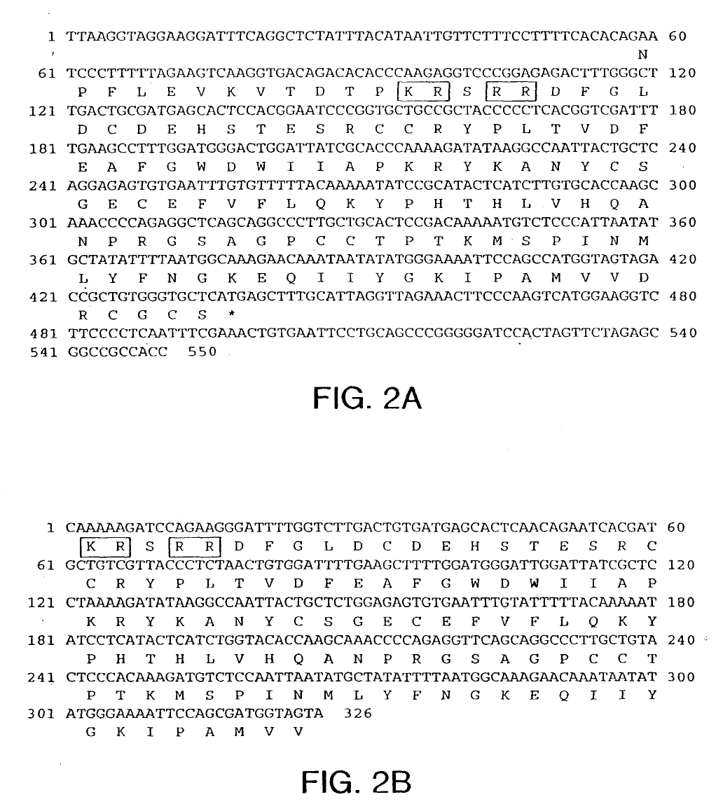

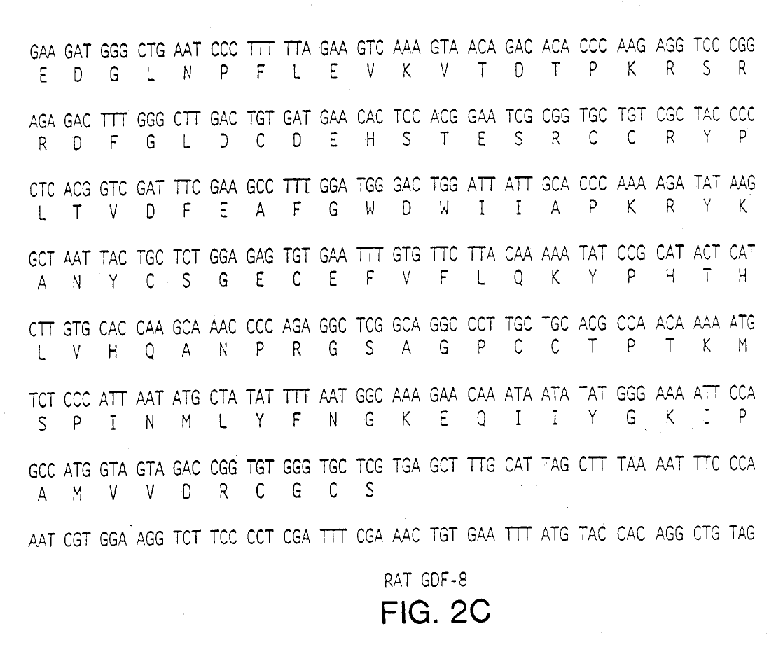

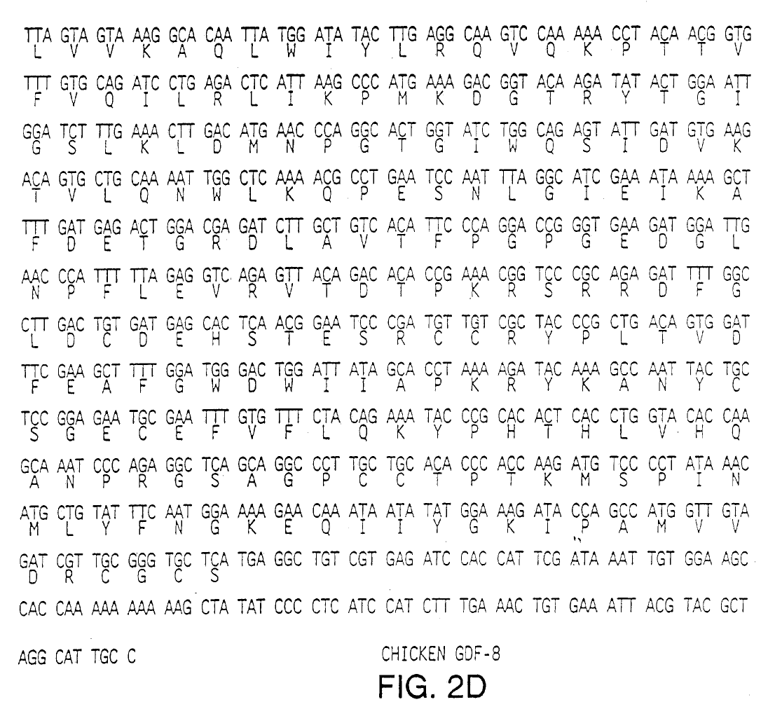

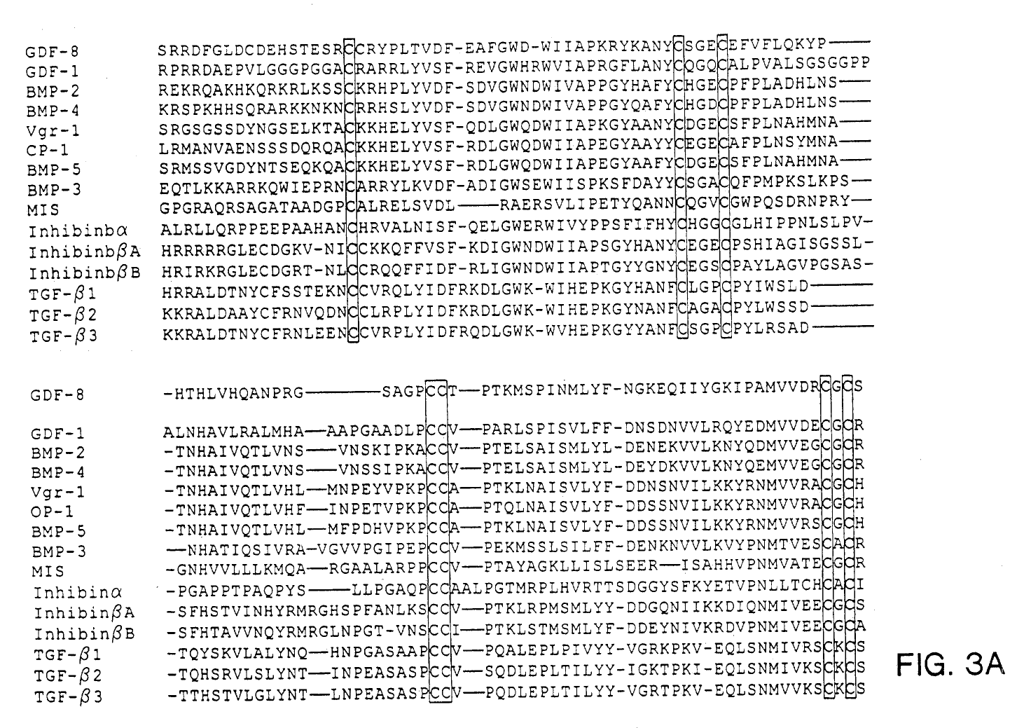

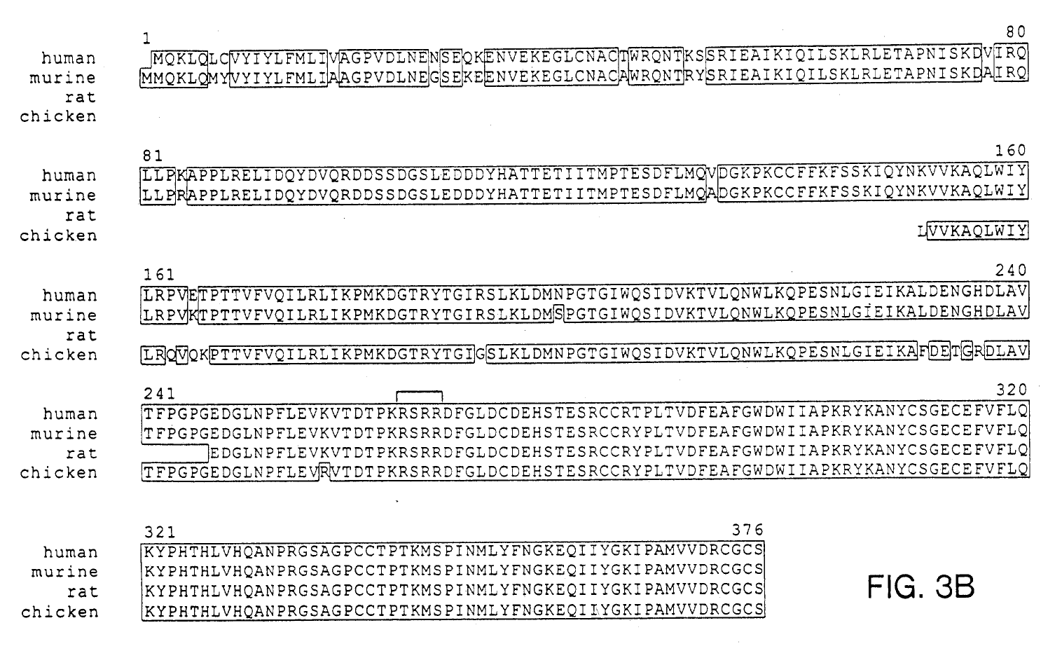

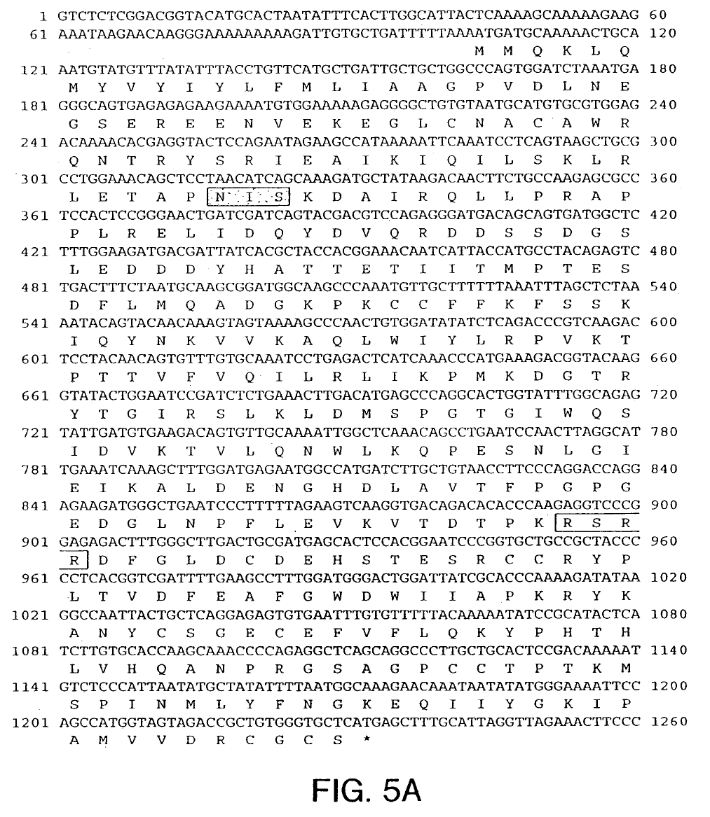

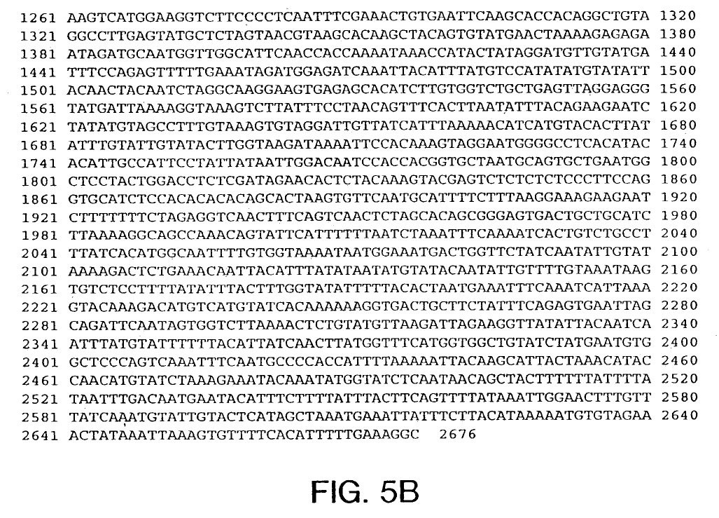

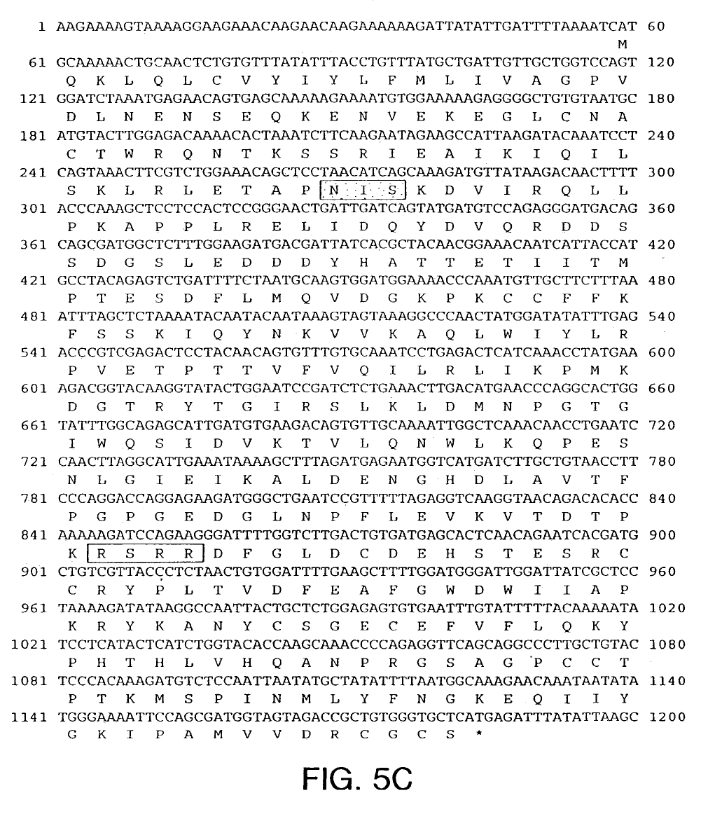

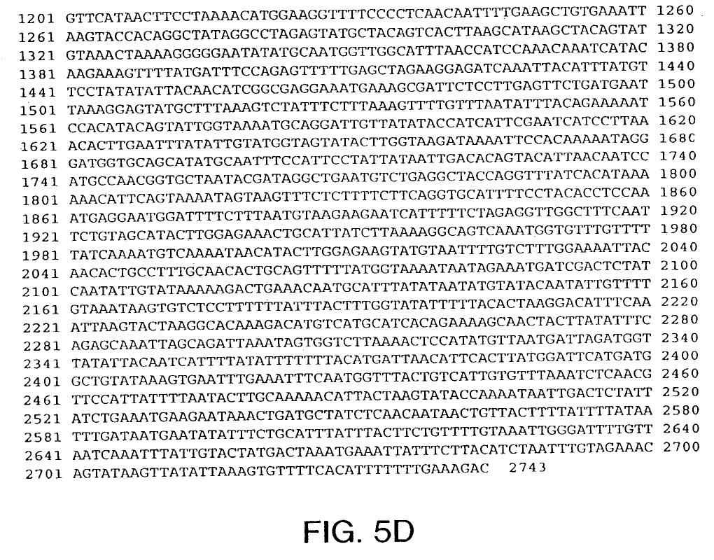

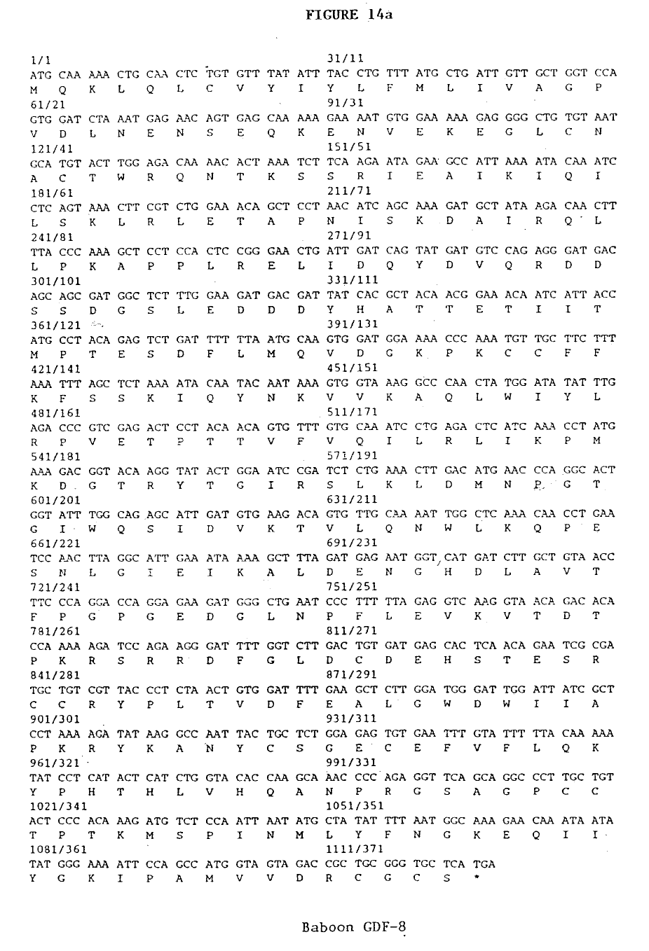

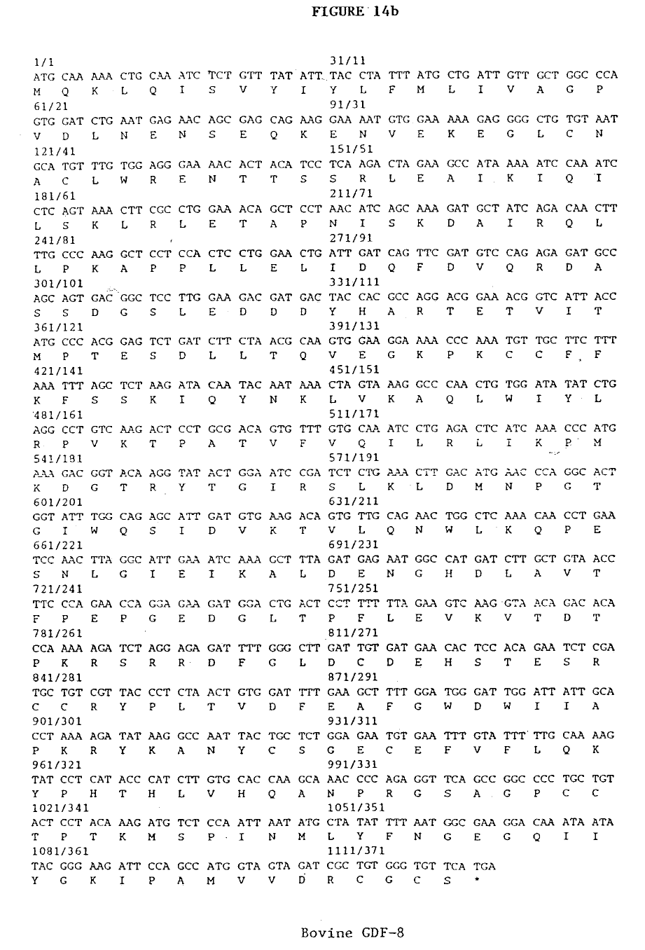

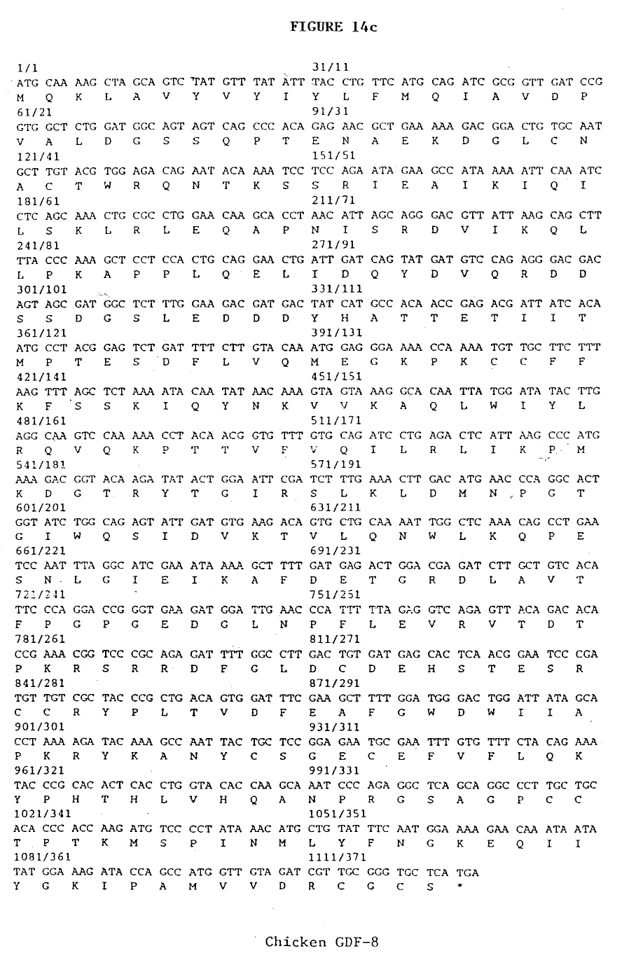

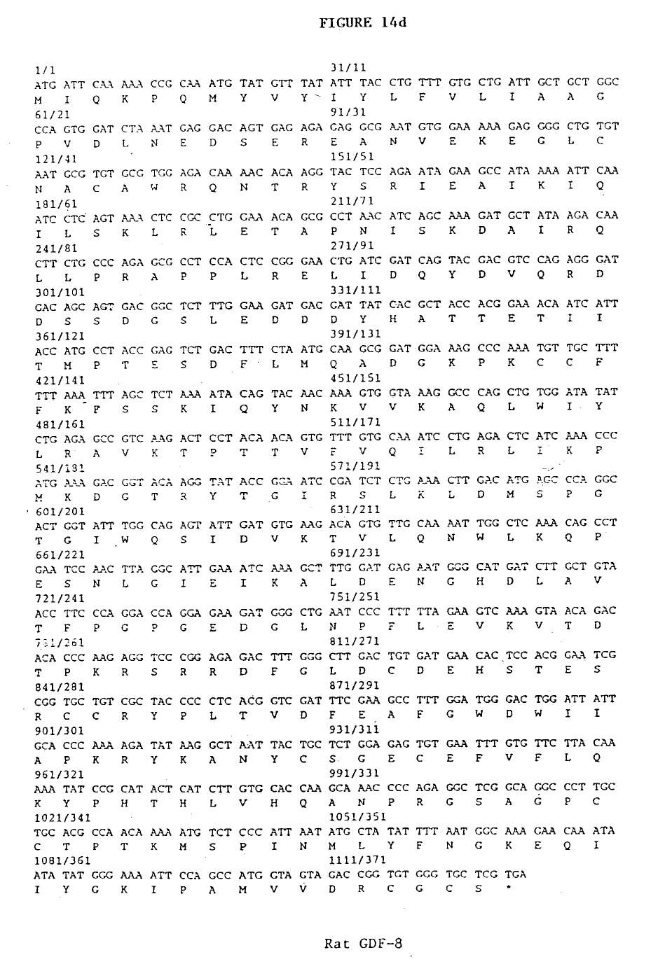

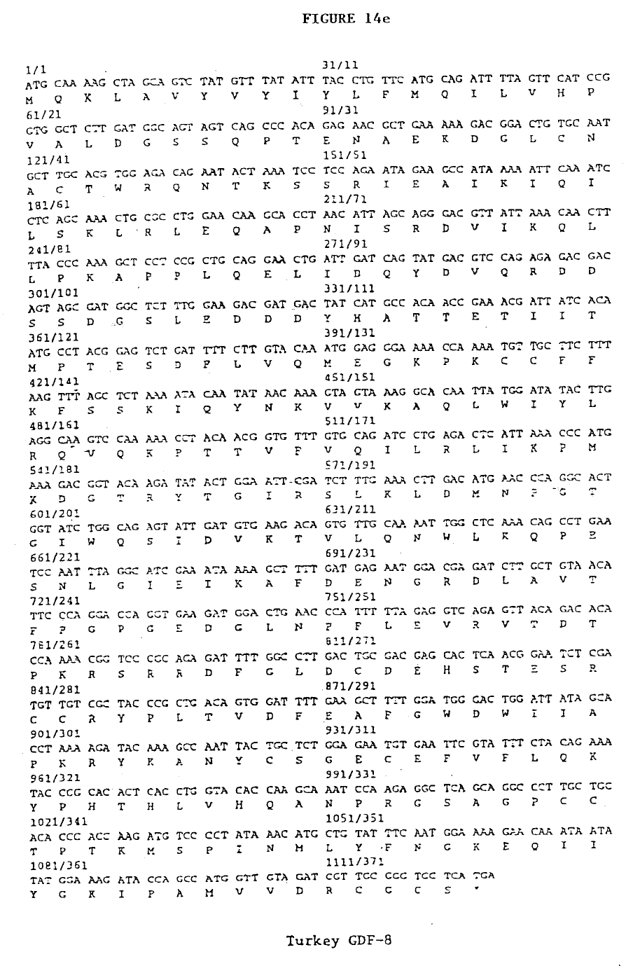

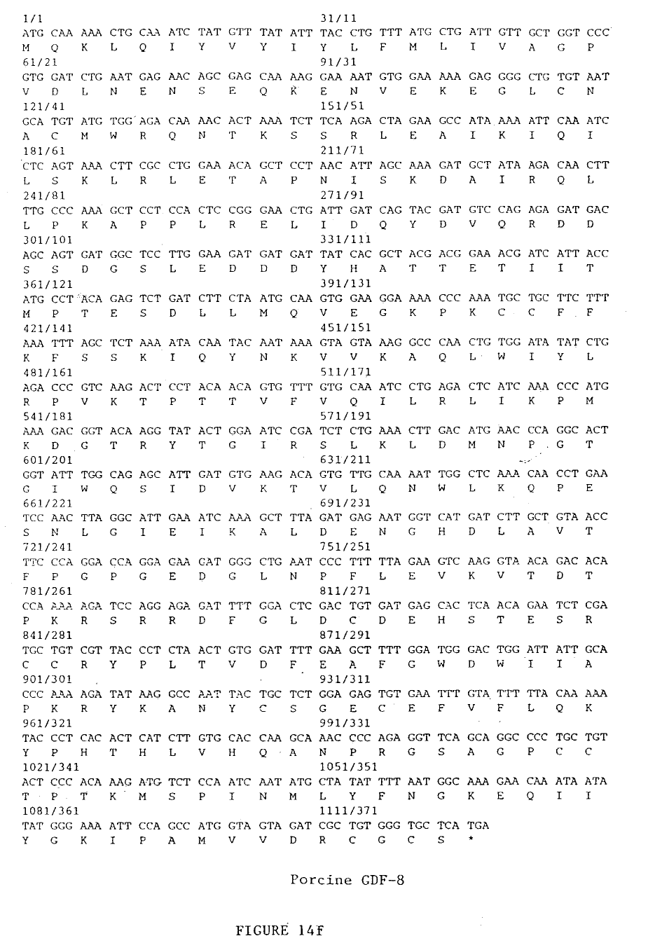

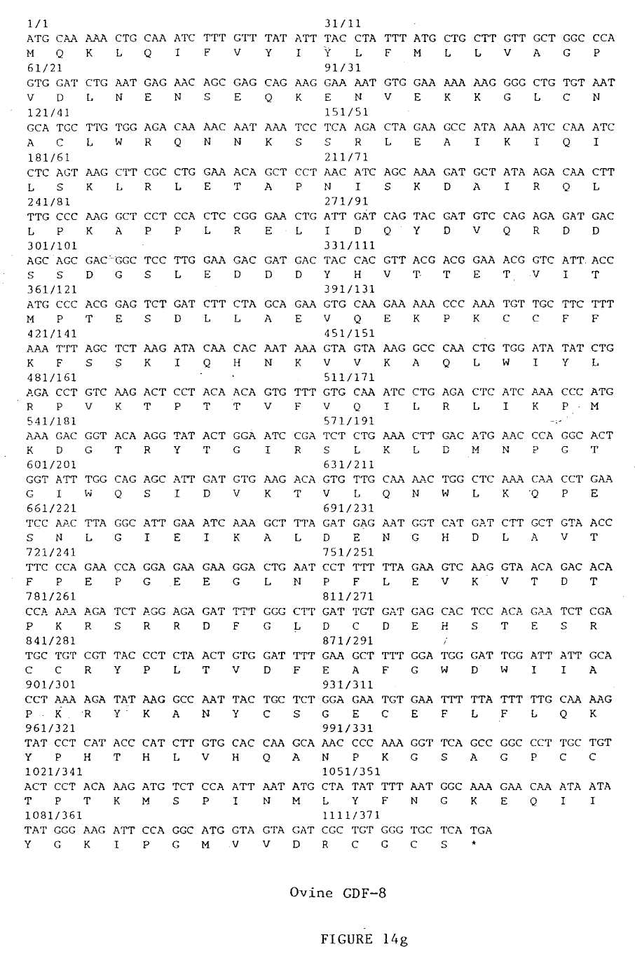

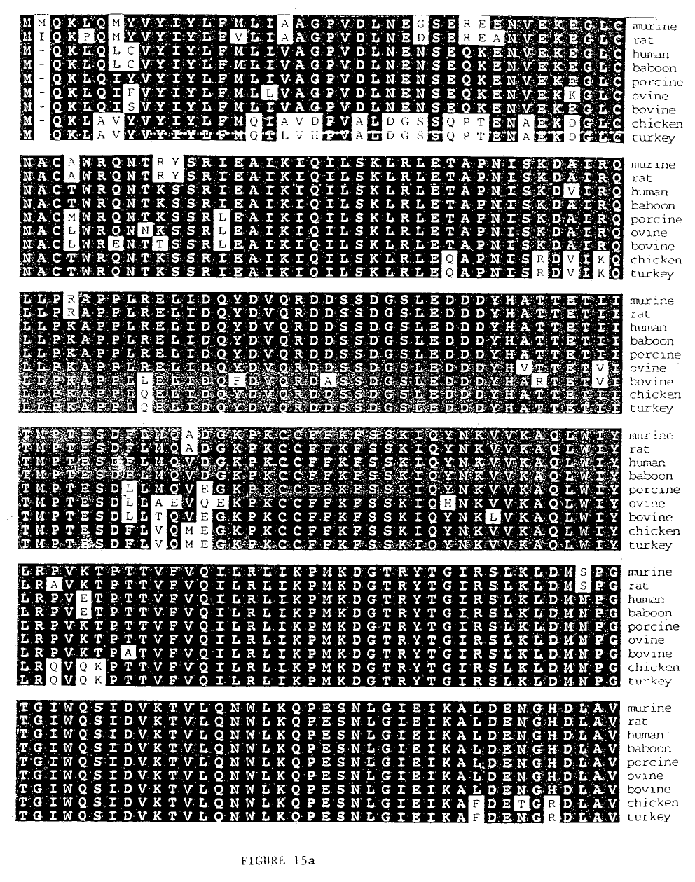

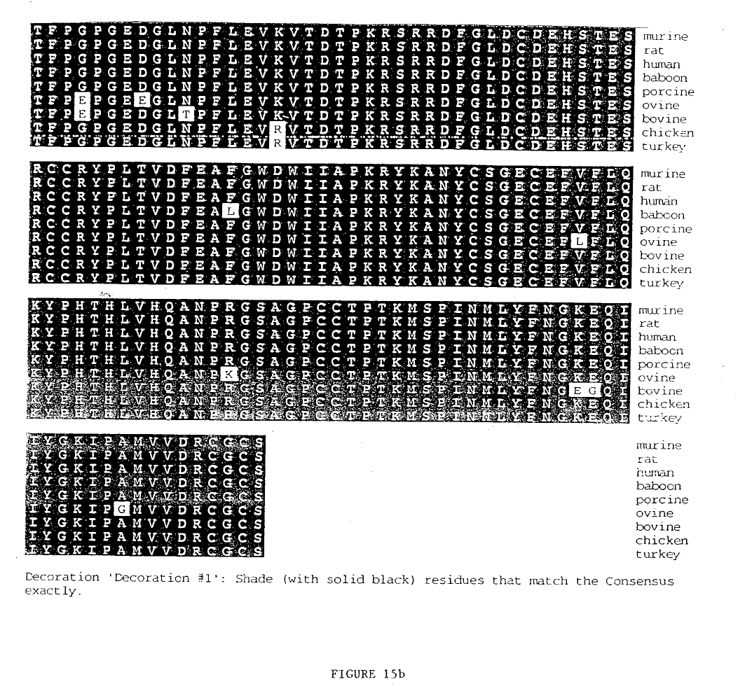

Also provided herein are the biologically active C-terminal fragments of chicken (FIG. 2C) and rat (FIG. 2D) GDF-8. The full length nucleotide and deduced amino acid sequences for baboon, bovine, chicken, rat, ovine, porcine, and turkey are shown in FIGS. 14A to 14G and human and murine are shown in FIG. 5. As shown in FIG. 3B, alignment of the amino acid sequences of human, murine, rat and chicken GDF-8 indicate that the sequences are 100% identical in the C-terminal biologically active fragment. FIGS. 15A and 15B also show the alignment of GDF-8 amino acid sequences for murine, rat, human, baboon, porcine, ovine, bovine, chicken and turkey. Given the extensive conservation of amino acid sequences between species, it would now be routine for one of skill in the art to obtain the GDF-8 nucleic acid and amino acid sequence for GDF-8 from any species, including those provided herein, as well as piscine, for example.

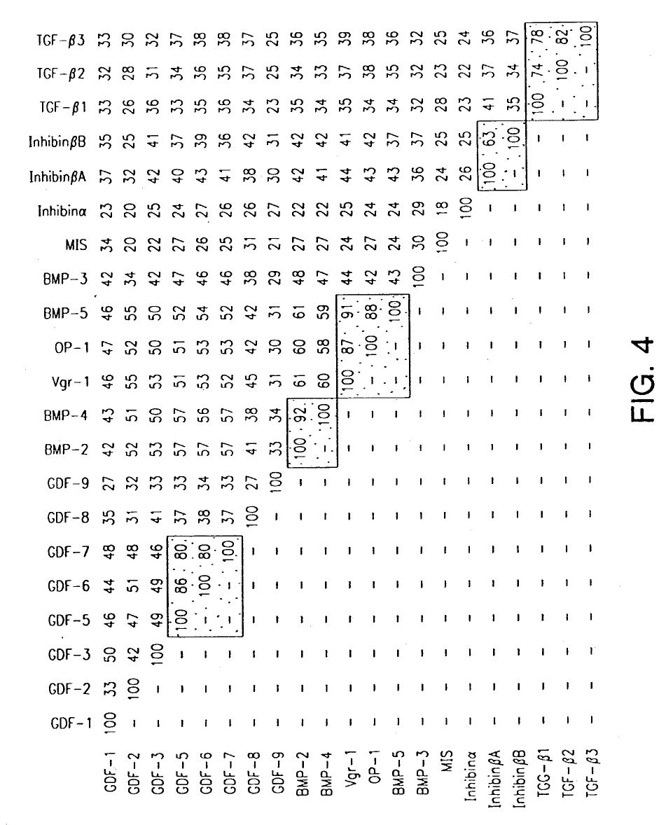

The C-terminal region of GDF-8 following the putative proteolytic processing site shows significant homology to the known members of the TGF-β superfamily. The GDF-8 sequence contains most of the residues that are highly conserved in other family members and in other species (see FIGS. 3A and 3B and 15A and 15B). Like the TGF-βs and inhibin βs, GDF-8 contains an extra pair of cysteine residues in addition to the 7 cysteines found in virtually all other family members. Among the known family members, GDF-8 is most homologous to Vgr-1 (45% sequence identity) (see FIG. 4).

Minor modifications of the recombinant GDF-8 primary amino acid sequence may result in proteins which have substantially equivalent activity as compared to the GDF-8 polypeptide described herein. Such modifications may be deliberate, as by site-directed mutagenesis, or may be spontaneous. All of the polypeptides produced by these modifications are included herein as long as the biological activity of GDF-8 still exists. Further, deletion of one or more amino acids can also result in a modification of the structure of the resultant molecule without significantly altering its biological activity. This can lead to the development of a smaller active molecule which would have broader utility. For example, one can remove amino or carboxy terminal amino acids which are not required for GDF-8 biological activity.

The nucleotide sequence encoding the GDF-8 polypeptide of the invention includes the disclosed sequence and conservative variations thereof The term “conservative variation” as used herein denotes the replacement of an amino acid residue by another, biologically similar residue. Examples of conservative variations include the substitution of one hydrophobic residue such as isoleucine, valine, leucine or methionine for another, or the substitution of one polar residue for another, such as the substitution of arginine for lysine, glutamic for aspartic acid, or glutamine for asparagine, and the like. The term “conservative variation” also includes the use of a substituted amino acid in place of an unsubstituted parent amino acid provided that antibodies raised to the substituted polypeptide also immunoreact with the unsubstituted polypeptide.

DNA sequences of the invention can be obtained by several methods. For example, the DNA can be isolated using hybridization techniques which are well known in the art. These include, but are not limited to: 1) hybridization of genomic or cDNA libraries with probes to detect homologous nucleotide sequences, 2) polymerase chain reaction (PCR) on genomic DNA or cDNA using primers capable of annealing to the DNA sequence of interest, and 3) antibody screening of expression libraries to detect cloned DNA fragments with shared structural features.

Preferably the GDF-8 polynucleotide of the invention is derived from a mammalian organism, and most preferably from mouse, rat, cow, pig, or human. GDF-8 polynucleotides from chicken, turkey, fish and other species are also included herein. Screening procedures which rely on nucleic acid hybridization make it possible to isolate any gene sequence from any organism, provided the appropriate probe is available. Given the extensive nucleotide and amino acid homology between species, it would be routine for one of skill in the art to obtain polynucleotides encoding GDF-8 from any species. Oligonucleotide probes, which correspond to a part of the sequence encoding the protein in question, can be synthesized chemically. This requires that short, oligopeptide stretches of amino acid sequence must be known. The DNA sequence encoding the protein can be deduced from the genetic code, however, the degeneracy of the code must be taken into account. It is possible to perform a mixed addition reaction when the sequence is degenerate. This includes a heterogeneous mixture of denatured double-stranded DNA. For such screening, hybridization is preferably performed on either single-stranded DNA or denatured double-stranded DNA. Hybridization is particularly useful in the detection of cDNA clones derived from sources where an extremely low amount of mRNA sequences relating to the polypeptide of interest are present. In other words, by using stringent hybridization conditions directed to avoid non-specific binding, it is possible, for example, to allow the autoradiographic visualization of a specific cDNA clone by the hybridization of the target DNA to that single probe in the mixture which is its complete complement (Wallace et al., Nucl. Acid Res. 9:879, 1981).

The development of specific DNA sequences encoding GDF-8 can also be obtained by: 1) isolation of double-stranded DNA sequences from the genomic DNA; 2) chemical manufacture of a DNA sequence to provide the necessary codons for the polypeptide of interest; and 3) in vitro synthesis of a double-stranded DNA sequence by reverse transcription of mRNA isolated from a eukaryotic donor cell. In the latter case, a double-stranded DNA complement of mRNA is eventually formed which is generally referred to as cDNA.

Of the three above-noted methods for developing specific DNA sequences for use in recombinant procedures, the isolation of genomic DNA isolates is the least common. This is especially true when it is desirable to obtain the microbial expression of mammalian polypeptides due to the presence of introns.

The synthesis of DNA sequences is frequently the method of choice when the entire sequence of amino acid residues of the desired polypeptide product is known. When the entire sequence of amino acid residues of the desired polypeptide is not known, the direct synthesis of DNA sequences is not possible and the method of choice is the synthesis of cDNA sequences. Among the standard procedures for isolating cDNA sequences of interest is the formation of plasmid- or phage-carrying cDNA libraries which are derived from reverse transcription of mRNA which is abundant in donor cells that have a high level of genetic expression. When used in combination with polymerase chain reaction technology, even rare expression products can be cloned. In those cases where significant portions of the amino acid sequence of the polypeptide are known, the production of labeled single or double-stranded DNA or RNA probe sequences duplicating a sequence putatively present in the target cDNA may be employed in DNA/DNA hybridization procedures which are carried out on cloned copies of the cDNA which have been denatured into a single-stranded form (Jay et al., Nucl. Acid Res., 11:2325, 1983).

A cDNA expression library, such as lambda gt11, can be screened indirectly for GDF-8 peptides having at least one epitope, using antibodies specific for GDF-8. Such antibodies can be either polyclonally or monoclonally derived and used to detect expression product indicative of the presence of GDF-8 cDNA.

In nucleic acid hybridization reactions, the conditions used to achieve a particular level of stringency will vary, depending on the nature of the nucleic acids being hybridized. For example, the length, degree of complementarity, nucleotide sequence composition (e.g., GC v. AT content), and nucleic acid type (e.g., RNA v. DNA) of the hybridizing regions of the nucleic acids can be considered in selecting hybridization conditions. An additional consideration is whether one of the nucleic acids is immobilized, for example, on a filter.

An example of progressively higher stringency conditions is as follows: 2×SSC/0.1% SDS at about room temperature (hybridization conditions); 0.2×SSC/0.1% SDS at about room temperature (low stringency conditions); 0.2×SSC/0.1% SDS at about 42° C. (moderate stringency conditions); and 0.1×SSC at about 68° C. (high stringency conditions). Washing can be carried out using only one of these conditions, e.g., high stringency conditions, or each of the conditions can be used, e.g., for 10-15 minutes each, in the order listed above, repeating any or all of the steps listed. However, as mentioned above, optimal conditions will vary, depending on the particular hybridization reaction involved, and can be determined empirically.

DNA sequences encoding GDF-8 can be expressed in vitro by DNA transfer into a suitable host cell. “Host cells” are cells in which a vector can be propagated and its DNA expressed. The term also includes any progeny of the subject host cell. It is understood that all progeny may not be identical to the parental cell since there may be mutations that occur during replication. However, such progeny are included when the term “host cell” is used. Methods of stable transfer, meaning that the foreign DNA is continuously maintained in the host, are known in the art.

In the present invention, the GDF-8 polynucleotide sequences may be inserted into a recombinant expression vector. The term “recombinant expression vector” refers to a plasmid, virus or other vehicle known in the art that has been manipulated by insertion or incorporation of the GDF-8 genetic sequences. Such expression vectors contain a promoter sequence which facilitates the efficient transcription of the inserted genetic sequence of the host. The expression vector typically contains an origin of replication, a promoter, as well as specific genes which allow phenotypic selection of the transformed cells. Vectors suitable for use in the present invention include, but are not limited to the T7-based expression vector for expression in bacteria (Rosenberg et al., Gene, 56:125, 1987), the pMSXND expression vector for expression in mammalian cells (Lee and Nathans, J. Biol. Chem., 263:3521, 1988) and baculovirus-derived vectors for expression in insect cells. The DNA segment can be present in the vector operably linked to regulatory elements, for example, a promoter (e.g., T7, metallothionein 1, or polyhedrin promoters).

Polynucleotide sequences encoding GDF-8 can be expressed in either prokaryotes or eukaryotes. Hosts can include microbial, yeast, insect and mammalian organisms. Methods of expressing DNA sequences having eukaryotic or viral sequences in prokaryotes are well known in the art. Biologically functional viral and plasmid DNA vectors capable of expression and replication in a host are known in the art. Such vectors are used to incorporate DNA sequences of the invention. Preferably, the mature C-terminal region of GDF-8 is expressed from a cDNA clone containing the entire coding sequence of GDF-8. Alternatively, the C-terminal portion of GDF-8 can be expressed as a fusion protein with the pro-region of another member of the TGF-β family or co-expressed with another pro-region (see for example, Hammonds et al., Molec. Endocrinol., 5:149, 1991; Gray and Mason, Science, 247:1328, 1990).

Transformation of a host cell with recombinant DNA may be carried out by conventional techniques as are well known to those skilled in the art. Where the host is prokaryotic, such as E. coli, competent cells which are capable of DNA uptake can be prepared from cells harvested after exponential growth phase and subsequently treated by the CaCl₂ method using procedures well known in the art. Alternatively, MgCl₂ or RbCl can be used. Transformation can also be performed after forming a protoplast of the host cell if desired.

When the host is a eukaryote, such methods of transfection of DNA as calcium phosphate co-precipitates, conventional mechanical procedures such as microinjection, electroporation, insertion of a plasmid encased in liposomes, or virus vectors may be used. Eukaryotic cells can also be cotransformed with DNA sequences encoding the GDF-8 of the invention, and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein. (see for example, Eukaryotic Viral Vectors, Cold Spring Harbor Laboratory, Gluzman ed., 1982).

Isolation and purification of microbial expressed polypeptide, or fragments thereof, provided by the invention, may be carried out by conventional means including preparative chromatography and immunological separations involving monoclonal or polyclonal antibodies.

GDF-8 Antibodies and Methods of Use

The invention includes antibodies immunoreactive with GDF-8 polypeptide or functional fragments thereof. Antibody which consists essentially of pooled monoclonal antibodies with different epitopic specificities, as well as distinct monoclonal antibody preparations are provided. Monoclonal antibodies are made from antigen containing fragments of the protein by methods well known to those skilled in the art (Kohler et al., Nature, 256:495, 1975). The term antibody as used in this invention is meant to include intact molecules as well as fragments thereof, such as Fab and F(ab′)₂, Fv and SCA fragments which are capable of binding an epitopic determinant on GDF-8.

(1) An Fab fragment consists of a monovalent antigen-binding fragment of an antibody molecule, and can be produced by digestion of a whole antibody molecule with the enzyme papain, to yield a fragment consisting of an intact light chain and a portion of a heavy chain.

(2) An Fab′ fragment of an antibody molecule can be obtained by treating a whole antibody molecule with pepsin, followed by reduction, to yield a molecule consisting of an intact light chain and a portion of a heavy chain. Two Fab′ fragments are obtained per antibody molecule treated in this manner.

(3) An (Fab′)₂ fragment of an antibody can be obtained by treating a whole antibody molecule with the enzyme pepsin, without subsequent reduction. A (Fab′)₂ fragment is a dimer of two Fab′ fragments, held together by two disulfide bonds.

(4) An Fv fragment is defined as a genetically engineered fragment containing the variable region of a light chain and the variable region of a heavy chain expressed as two chains.

(5) A single chain antibody (“SCA”) is a genetically engineered single chain molecule containing the variable region of a light chain and the variable region of a heavy chain, linked by a suitable, flexible polypeptide linker.

As used in this invention, the term “epitope” refers to an antigenic determinant on an antigen, such as a GDF-8 polypeptide, to which the paratope of an antibody, such as an GDF-8-specific antibody, binds. Antigenic determinants usually consist of chemically active surface groupings of molecules, such as amino acids or sugar side chains, and can have specific three-dimensional structural characteristics, as well as specific charge characteristics.

As is mentioned above, antigens that can be used in producing GDF-8-specific antibodies include GDF-8 polypeptides or GDF-8 polypeptide fragments. The polypeptide or peptide used to immunize an animal can be obtained by standard recombinant, chemical synthetic, or purification methods. As is well known in the art, in order to increase immunogenicity, an antigen can be conjugated to a carrier protein. Commonly used carriers include keyhole limpet hemocyanin (KLH), thyroglobulin, bovine serum albumin (BSA), and tetanus toxoid. The coupled peptide is then used to immunize the animal (e.g., a mouse, a rat, or a rabbit). In addition to such carriers, well known adjuvants can be administered with the antigen to facilitate induction of a strong immune response.

The term “cell-proliferative disorder” denotes malignant as well as non-malignant cell populations which often appear to differ from the surrounding tissue both morphologically and genotypically. Malignant cells (i.e. cancer) develop as a result of a multistep process. The GDF-8 polynucleotide that is an antisense molecule or that encodes a dominant negative GDF-8 is useful in treating malignancies of the various organ systems, particularly, for example, cells in muscle, bone, kidney or adipose tissue. Essentially, any disorder which is etiologically linked to altered expression of GDF-8 could be considered susceptible to treatment with a GDF-8 agent (e.g., a suppressing or enhancing agent). One such disorder is a malignant cell proliferative disorder, for example.

The invention provides a method for detecting a cell proliferative disorder of muscle, bone, kidney or adipose tissue which comprises contacting an anti-GDF-8 antibody with a cell suspected of having a GDF-8 associated disorder and detecting binding to the antibody. The antibody reactive with GDF-8 is labeled with a compound which allows detection of binding to GDF-8. For purposes of the invention, an antibody specific for GDF-8 polypeptide may be used to detect the level of GDF-8 in biological fluids and tissues. Any specimen containing a detectable amount of antigen can be used. Preferred samples of this invention include muscle, bone or kidney tissue. The level of GDF-8 in the suspect cell can be compared with the level in a normal cell to determine whether the subject has a GDF-8-associated cell proliferative disorder. Such methods of detection are also useful using nucleic acid hybridization to detect the level of GDF-8 mRNA in a sample or to detect an altered GDF-8 gene. Preferably the subject is human.

The antibodies of the invention can be used in any subject in which it is desirable to administer in vitro or in vivo immunodiagnosis or immunotherapy. The antibodies of the invention are suited for use, for example, in immunoassays in which they can be utilized in liquid phase or bound to a solid phase carrier. In addition, the antibodies in these immunoassays can be detectably labeled in various ways. Examples of types of immunoassays which can utilize antibodies of the invention are competitive and non-competitive immunoassays in either a direct or indirect format. Examples of such immunoassays are the radioimmunoassay (RIA) and the sandwich (immunometric) assay. Detection of the antigens using the antibodies of the invention can be done utilizing immunoassays which are run in either the forward, reverse, or simultaneous modes, including immunohistochemical assays on physiological samples. Those of skill in the art will know, or can readily discern, other immunoassay formats without undue experimentation.

The antibodies of the invention can be bound to many different carriers and used to detect the presence of an antigen comprising the polypeptide of the invention. Examples of well-known carriers include glass, polystyrene, polypropylene, polyethylene, dextran, nylon, amylases, natural and modified celluloses, polyacrylamides, agaroses and magnetite. The nature of the carrier can be either soluble or insoluble for purposes of the invention. Those skilled in the art will know of other suitable carriers for binding antibodies, or will be able to ascertain such, using routine experimentation.

There are many different labels and methods of labeling known to those of ordinary skill in the art. Examples of the types of labels which can be used in the present invention include enzymes, radioisotopes, fluorescent compounds, colloidal metals, chemiluminescent compounds, phosphorescent compounds, and bioluminescent compounds. Those of ordinary skill in the art will know of other suitable labels for binding to the antibody, or will be able to ascertain such, using routine experimentation.

Another technique which may also result in greater sensitivity consists of coupling the antibodies to low molecular weight haptens. These haptens can then be specifically detected by means of a second reaction. For example, it is common to use such haptens as biotin, which reacts with avidin, or dinitrophenyl, puridoxal, and fluorescein, which can react with specific antihapten antibodies.

In using the monoclonal antibodies of the invention for the in vivo detection of antigen, the detectably labeled antibody is given a dose which is diagnostically effective. The term “diagnostically effective” means that the amount of detectably labeled monoclonal antibody is administered in sufficient quantity to enable detection of the site having the antigen comprising a polypeptide of the invention for which the monoclonal antibodies are specific.

The concentration of detectably labeled monoclonal antibody which is administered should be sufficient such that the binding to those cells having the polypeptide is detectable compared to the background. Further, it is desirable that the detectably labeled monoclonal antibody be rapidly cleared from the circulatory system in order to give the best target-to-background signal ratio.

As a rule, the dosage of detectably labeled monoclonal antibody for in vivo diagnosis will vary depending on such factors as age, sex, and extent of disease of the individual. Such dosages may vary, for example, depending on whether multiple injections are given, antigenic burden, and other factors known to those of skill in the art.

For in vivo diagnostic imaging, the type of detection instrument available is a major factor in selecting a given radioisotope. The radioisotope chosen must have a type of decay which is detectable for a given type of instrument. Still another important factor in selecting a radioisotope for in vivo diagnosis is that deleterious radiation with respect to the host is minimized. Ideally, a radioisotope used for in vivo imaging will lack a particle emission, but produce a large number of photons in the 140-250 keV range, which may readily be detected by conventional gamma cameras.

For in vivo diagnosis radioisotopes may be bound to immunoglobulin either directly or indirectly by using an intermediate functional group. intermediate functional groups which often are used to bind radioisotopes which exist as metallic ions to immunoglobulins are the bifunctional chelating agents such as diethylenetriaminepentacetic acid (DTPA) and ethylenediaminetetraacetic acid (EDTA) and similar molecules. Typical examples of metallic ions which can be bound to the monoclonal antibodies of the invention are ¹¹¹In, ⁹⁷Ru, ⁶⁷Ga, ⁶⁸Ga, ⁷²As, ⁸⁹Zr and ²⁰¹Tl.

The monoclonal antibodies of the invention can also be labeled with a paramagnetic isotope for purposes of in vivo diagnosis, as in magnetic resonance imaging (MRI) or electron spin resonance (ESR). In general, any conventional method for visualizing diagnostic imaging can be utilized. Usually gamma and positron emitting radioisotopes are used for camera imaging and paramagnetic isotopes for MRI. Elements which are particularly useful in such techniques include ¹⁵⁷Gd, ⁵⁵Mn, ¹⁶²Dy, ⁵²Cr, and ⁵⁶Fe.

The monoclonal antibodies of the invention can be used in vitro and in vivo to monitor the course of amelioration of a GDF-8-associated disease in a subject. Thus, for example, by measuring the increase or decrease in the number of cells expressing antigen comprising a polypeptide of the invention or changes in the concentration of such antigen present in various body fluids, it would be possible to determine whether a particular therapeutic regimen aimed at ameliorating the GDF-8-associated disease is effective. The term “ameliorate” denotes a lessening of the detrimental effect of the GDF-8-associated disease in the subject receiving therapy.

Additional Methods of Treatment and Diagnosis

The present invention identifies a nucleotide sequence that can be expressed in an altered manner as compared to expression in a normal cell, therefore it is possible to design appropriate therapeutic or diagnostic techniques directed to this sequence. Treatment includes administration of a reagent which modulates activity. The term “modulate” envisions the suppression or expression of GDF-8 when it is over-expressed, or augmentation of GDF-8 expression when it is underexpressed. When a muscle or bone-associated disorder is associated with GDF-8 overexpression, such suppressive reagents as antisense GDF-8 polynucleotide sequence, dominant negative sequences or GDF-8 binding antibody can be introduced into a cell. In addition, an anti-idiotype antibody which binds to a monoclonal antibody which binds GDF-8 of the invention, or an epitope thereof, may also be used in the therapeutic method of the invention. Alternatively, when a cell proliferative disorder is associated with underexpression or expression of a mutant GDF-8 polypeptide, a sense polynucleotide sequence (the DNA coding strand) or GDF-8 polypeptide can be introduced into the cell. Such muscle or bone-associated disorders include cancer, muscular dystrophy, spinal cord injury, traumatic injury, congestive obstructive pulmonary disease (COPD), AIDS or cachexia. In addition, the method of the invention can be used in the treatment of obesity or of disorders related to abnormal proliferation of adipocytes. One of skill in the art can determine whether or not a particular therapeutic course of treatment is successful by several methods described herein (e.g., muscle fiber analysis or biopsy; determination of fat content). The present examples demonstrate that the methods of the invention are useful for decreasing fat content, and therefore would be useful in the treatment of obesity and related disorders (e.g., diabetes). Neurodegenerative disorders are also envisioned as treated by the method of the invention.

Thus, where a cell-proliferative disorder is associated with the expression of GDF-8, nucleic acid sequences that interfere with GDF-8 expression at the translational level can be used. This approach utilizes, for example, antisense nucleic acid and ribozymes to block translation of a specific GDF-8 mRNA, either by masking that mRNA with an antisense nucleic acid or by cleaving it with a ribozyme. Such disorders include neurodegenerative diseases, for example. In addition, dominant-negative GDF-8 mutants would be useful to actively interfere with function of “normal” GDF-8.

Antisense nucleic acids are DNA or RNA molecules that are complementary to at least a portion of a specific mRNA molecule (Weintraub, Scientific American, 262:40, 1990). In the cell, the antisense nucleic acids hybridize to the corresponding mRNA, forming a double-stranded molecule. The antisense nucleic acids interfere with the translation of the mRNA, since the cell will not translate a mRNA that is double-stranded.

Antisense oligomers of about 15 nucleotides are preferred, since they are easily synthesized and are less likely to cause problems than larger molecules when introduced into the target GDF-8-producing cell. The use of antisense methods to inhibit the in vitro translation of genes is well known in the art (Marcus-Sakura, Anal. Biochem., 172:289, 1988).

Ribozymes are RNA molecules possessing the ability to specifically cleave other single-stranded RNA in a manner analogous to DNA restriction endonucleases. Through the modification of nucleotide sequences which encode these RNAs, it is possible to engineer molecules that recognize specific nucleotide sequences in an RNA molecule and cleave it (Cech, J. Amer Med. Assn., 260:3030, 1988). A major advantage of this approach is that, because they are sequence-specific, only mRNAs with particular sequences are inactivated.

There are two basic types of ribozymes namely, tetrahymena-type (Hasselhoff, Nature, 334:585, 1988) and “hammerhead”-type. Tetrahymena-type ribozymes recognize sequences which are four bases in length, while “hammerhead”-type ribozymes recognize base sequences 11-18 bases in length. The longer the recognition sequence, the greater the likelihood that the sequence will occur exclusively in the target mRNA species. Consequently, hammerhead-type ribozymes are preferable to tetrahymena-type ribozymes for inactivating a specific mRNA species and 18-based recognition sequences are preferable to shorter recognition sequences.

In another embodiment of the present invention, a nucleotide sequence encoding a GDF-8 dominant negative protein is provided. For example, a genetic construct that contain such a dominant negative encoding gene may be operably linked to a promoter, such as a tissue-specific promoter. For example, a skeletal muscle specific promoter (e.g., human skeletal muscle α-actin promoter) or developmentally specific promoter (e.g., MyHC 3, which is restricted in skeletal muscle to the embryonic period of development, or an inducible promoter (e.g., the orphan nuclear receptor TIS1).

Such constructs are useful in methods of modulating a subject's skeletal mass. For example, a method include transforming an organism, tissue, organ or cell with a genetic construct encoding a dominant negative GDF-8 protein and suitable promoter in operable linkage and expressing the dominant negative encoding GDF-8 gene, thereby modulating muscle and/or bone mass by interfering with wild-type GDF-8 activity.

GDF-8 most likely forms dimers, homodimers or heterodimers and may even form heterodimers with other GDF family members, such as GDF-11 (see Example 4). Hence, while not wanting to be bound by a particular theory, Hence, while not wanting to be bound by a particular theory, the dominant negative effect described herein may involve the formation of non-functional homodimers or heterodimers of dominant negative and wild-type GDF-8 monomers. More specifically, it is possible that any non-functional homodimer or any heterodimer formed by the dimerization of wild-type and/or dominant negative GDF-8 monomers produces a dominant effect by: 1) being synthesized but not processed or secreted; 2) inhibiting the secretion of wild type GDF-8; 3) preventing normal proteolytic cleavage of the pre-protein thereby producing a nonfunctional GDF-8 molecule; 4) altering the affinity of the non-functional dimer (e.g., homodimeric or heterodimeric GDF-8) to a receptor or generating an antagonistic form of GDF-8 that binds a receptor without activating it; or 5) inhibiting the intracellular processing or secretion of GDF-8 related or TGF-β family proteins.

Non-functional GDF-8 can function to inhibit the growth regulating actions of GDF-8 on muscle cells that include a dominant negative GDF-8 gene. Deletion or missense dominant negative forms of GDF-8 that retain the ability to form dimers with wild-type GDF-8 protein but do not function as wild-type GDF-8 proteins may be used to inhibit the biological activity of endogenous wild- type GDF-8. For example, in one embodiment, the proteolytic processing site of GDF-8 may be altered (e.g., deleted) resulting in a GDF-8 molecule able to undergo subsequent dimerization with endogenous wild-type GDF-8 but unable to undergo further processing into a mature GDF-8 form. Alternatively, a non-functional GDF-8 can function as a monomeric species to inhibit the growth regulating actions of GDF-8 on muscle cells.

Any genetic recombinant method in the art may be used, for example, recombinant viruses may be engineered to express a dominant negative form of GDF-8 which may be used to inhibit the activity of wild-type GDF-8. Such viruses may be used therapeutically for treatment of diseases resulting from aberrant over-expression or activity of GDF-8 protein, such as in denervation hypertrophy or as a means of controlling GDF-8 expression when treating disease conditions involving muscle, such as in musculodegenerative diseases or in tissue repair due to trauma or in modulating GDF-8 expression in animal husbandry (e.g., transgenic animals for agricultural purposes).

The invention provides a method for treating a muscle, bone, kidney (chronic or acute) or adipose tissue disorder in a subject. The method includes administering a therapeutically effective amount of a GDF-8 agent to the subject, thereby inhibiting abnormal growth of muscle, bone, kidney or adipose tissue. The GDF-8 agent may include a GDF-8 antisense molecule or a dominant negative polypeptide, for example. A “therapeutically effective amount” of a GDF-8 agent is that amount that ameliorates symptoms of the disorder or inhibits GDF-8 induced growth of muscle or bone, for example, as compared with a normal subject.

Gene Therapy

The present invention also provides gene therapy for the treatment of cell proliferative or immunologic disorders which are mediated by GDF-8 protein. Such therapy would achieve its therapeutic effect by introduction of the GDF-8 antisense or dominant negative encoding polynucleotide into cells having the proliferative disorder. Delivery of antisense or dominant negative GDF-8 polynucleotide can be achieved using a recombinant expression vector such as a chimeric virus or a colloidal dispersion system. Especially preferred for therapeutic delivery of antisense or dominant negative sequences is the use of targeted liposomes. In contrast, when it is desirable to enhance GDF-8 production, a “sense” GDF-8 polynucleotide or functional equivalent (e.g., the C-term active region) is introduced into the appropriate cell(s).

Various viral vectors which can be utilized for gene therapy as taught herein include adenovirus, herpes virus, vaccinia, or, preferably, an RNA virus such as a retrovirus. Preferably, the retroviral vector is a derivative of a murine or avian retrovirus. Examples of retroviral vectors in which a single foreign gene can be inserted include, but are not limited to: Moloney murine leukemia virus (MoMuLV), Harvey murine sarcoma virus (HaMuSV), murine mammary tumor virus (MuMTV), and Rous Sarcoma Virus (RSV). A number of additional retroviral vectors can incorporate multiple genes. All of these vectors can transfer or incorporate a gene for a selectable marker so that transduced cells can be identified and generated. By inserting a GDF-8 sequence of interest into the viral vector, along with another gene which encodes the ligand for a receptor on a specific target cell, for example, the vector is now target specific. Retroviral vectors can be made target specific by attaching, for example, a sugar, a glycolipid, or a protein. Preferred targeting is accomplished by using an antibody to target the retroviral vector. Those of skill in the art will know of, or can readily ascertain without undue experimentation, specific polynucleotide sequences which can be inserted into the retroviral genome or attached to a viral envelope to allow target specific delivery of the retroviral vector containing the GDF-8 antisense polynucleotide.

Since recombinant retroviruses are defective, they require assistance in order to produce infectious vector particles. This assistance can be provided, for example, by using helper cell lines that contain plasmids encoding all of the structural genes of the retrovirus under the control of regulatory sequences within the LTR. These plasmids are missing a nucleotide sequence which enables the packaging mechanism to recognize an RNA transcript for encapsulation. Helper cell lines which have deletions of the packaging signal include, but are not limited to ψ2, PA317 and PA12, for example. These cell lines produce empty virions, since no genome is packaged. If a retroviral vector is introduced into such cells in which the packaging signal is intact, but the structural genes are replaced by other genes of interest, the vector can be packaged and vector virion produced.

Alternatively, NIH 3T3 or other tissue culture cells can be directly transfected with plasmids encoding the retroviral structural genes gag, pol and env, by conventional calcium phosphate transfection. These cells are then transfected with the vector plasmid containing the genes of interest. The resulting cells release the retroviral vector into the culture medium.

Another targeted delivery system for GDF-8 polynucleotides is a colloidal dispersion system. Colloidal dispersion systems include macromolecule complexes, nanocapsules, microspheres, beads, and lipid-based systems including oil-in-water emulsions, micelles, mixed micelles, and liposomes. The preferred colloidal system of this invention is a liposome. Liposomes are artificial membrane vesicles which are useful as delivery vehicles in vitro and in vivo. It has been shown that large unilamellar vesicles (LUV), which range in size from 0.2-4.0 μm can encapsulate a substantial percentage of an aqueous buffer containing large macromolecules. RNA, DNA and intact virions can be encapsulated within the aqueous interior and be delivered to cells in a biologically active form (Fraley et al., Trends Biochem. Sci., 6:77, 1981). In addition to mammalian cells, liposomes have been used for delivery of polynucleotides in plant, yeast and bacterial cells. In order for a liposome to be an efficient gene transfer vehicle, the following characteristics should be present: (1) encapsulation of the genes of interest at high efficiency while not compromising their biological activity; (2) preferential and substantial binding to a target cell in comparison to non-target cells; (3) delivery of the aqueous contents of the vesicle to the target cell cytoplasm at high efficiency; and (4) accurate and effective expression of genetic information (Manning et al., BioTechniques, 6:682, 1988).

The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations.

Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Particularly useful are diacylphosphatidylglycerols, where the lipid moiety contains from 14-18 carbon atoms, particularly from 16-18 carbon atoms, and is saturated. Illustrative phospholipids include egg phosphatidylcholine, dipalmitoylphosphatidylcholine and distearoylphosphatidylcholine.

The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

The surface of the targeted delivery system may be modified in a variety of ways. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand.

Due to the expression of GDF-8 in muscle, bone, kidney and adipose tissue, there are a variety of applications using the polypeptide, polynucleotide, and antibodies of the invention, related to these tissues. Such applications include treatment of cell proliferative disorders involving these and other tissues, such as neural tissue. In addition, GDF-8 may be useful in various gene therapy procedures. In embodiments where GDF-8 polypeptide is administered to a subject, the dosage range is about 0.1 μg/kg to 100 mg/kg; more preferably from about 1 μg/kg to 75 mg/kg and most preferably from about 10 mg/kg to 50 mg/kg.

Chromosomal Location of GDF-8

The data in Example 6 shows that the human GDF-8 gene is located on chromosome 2. By comparing the chromosomal location of GDF-8 with the map positions of various human disorders, it should be possible to determine whether mutations in the GDF-8 gene are involved in the etiology of human diseases. For example, an autosomal recessive form of juvenile amyotrophic lateral sclerosis has been shown to map to chromosome 2 (Hentati et al., Neurology, 42 {Suppl. 3}:201, 1992). More precise mapping of GDF-8 and analysis of DNA from these patients may indicate that GDF-8 is, in fact, the gene affected in this disease. In addition, GDF-8 is useful for distinguishing chromosome 2 from other chromosomes.

Transgenic Animals and Methods of Making the Same

Various methods to make the transgenic animals of the subject invention can be employed. Generally speaking, three such methods may be employed. In one such method, an embryo at the pronuclear stage (a “one cell embryo”) is harvested from a female and the transgene is microinjected into the embryo, in which case the transgene will be chromosomally integrated into both the germ cells and somatic cells of the resulting mature animal. In another such method, embryonic stem cells are isolated and the transgene incorporated therein by electroporation, plasmid transfection or microinjection, followed by reintroduction of the stem cells into the embryo where they colonize and contribute to the germ line. Methods for microinjection of mammalian species is described in U.S. Pat. No. 4,873,191. In yet another such method, embryonic cells are infected with a retrovirus containing the transgene whereby the germ cells of the embryo have the transgene chromosomally integrated therein. When the animals to be made transgenic are avian, because avian fertilized ova generally go through cell division for the first twenty hours in the oviduct, microinjection into the pronucleus of the fertilized egg is problematic due to the inaccessibility of the pronucleus. Therefore, of the methods to make transgenic animals described generally above, retrovirus infection is preferred for avian species, for example as described in U.S. Pat. No. 5,162,215. If microinjection is to be used with avian species, however, a recently published procedure by Love et al., (BioTechnology, 12, Jan. 1994) can be utilized whereby the embryo is obtained from a sacrificed hen approximately two and one-half hours after the laying of the previous laid egg, the transgene is microinjected into the cytoplasm of the germinal disc and the embryo is cultured in a host shell until maturity. When the animals to be made transgenic are bovine or porcine, microinjection can be hampered by the opacity of the ova thereby making the nuclei difficult to identify by traditional differential interference-contrast microscopy. To overcome this problem, the ova can first be centrifuged to segregate the pronuclei for better visualization.

The “non-human animals” of the invention bovine, porcine, ovine and avian animals (e.g., cow, pig, sheep, chicken, turkey). The “transgenic non-human animals” of the invention are produced by introducing “transgenes” into the germline of the non-human animal. Embryonal target cells at various developmental stages can be used to introduce transgenes. Different methods are used depending on the stage of development of the embryonal target cell. The zygote is the best target for micro-injection. The use of zygotes as a target for gene transfer has a major advantage in that in most cases the injected DNA will be incorporated into the host gene before the first cleavage (Brinster et al., Proc. Nati. Acad. Sci. USA 82:4438-4442, 1985). As a consequence, all cells of the transgenic non-human animal will carry the incorporated transgene. This will in general also be reflected in the efficient transmission of the transgene to offspring of the founder since 50% of the germ cells will harbor the transgene.

The term “transgenic” is used to describe an animal which includes exogenous genetic material within all of its cells. A “transgenic” animal can be produced by cross-breeding two chimeric animals which include exogenous genetic material within cells used in reproduction. Twenty-five percent of the resulting offspring will be transgenic, i.e., animals which include the exogenous genetic material within all of their cells in both alleles. 50% of the resulting animals will include the exogenous genetic material within one allele and 25% will include no exogenous genetic material.

In the microinjection method useful in the practice of the subject invention, the transgene is digested and purified free from any vector DNA e.g. by gel electrophoresis. It is preferred that the transgene include an operatively associated promoter which interacts with cellular proteins involved in transcription, ultimately resulting in constitutive expression. Promoters useful in this regard include those from cytomegalovirus (CMV), Moloney leukemia virus (MLV), and herpes virus, as well as those from the genes encoding metallothionein, skeletal actin, P-enolpyruvate carboxylase (PEPCK), phosphoglycerate (PGK), DHFR, and thymidine kinase. Promoters for viral long terminal repeats (LTRs) such as Rous Sarcoma Virus can also be employed. When the animals to be made transgenic are avian, preferred promoters include those for the chicken β-globin gene, chicken lysozyme gene, and avian leukosis virus. Constructs useful in plasmid transfection of embryonic stem cells will employ additional regulatory elements well known in the art such as enhancer elements to stimulate transcription, splice acceptors, termination and polyadenylation signals, and ribosome binding sites to permit translation.

Retroviral infection can also be used to introduce transgene into a non-human animal, as described above. The developing non-human embryo can be cultured in vitro to the blastocyst stage. During this time, the blastomeres can be targets for retro viral infection (Jaenich, Proc. Natl. Acad. Sci USA 73:1260-1264, 1976). Efficient infection of the blastomeres is obtained by enzymatic treatment to remove the zona pellucida (Hogan et al. (1986) in Manipulating the Mouse Embryo, Cold Spring Harbor Laboratory Press, Cold Spring Harbor, N.Y). The viral vector system used to introduce the transgene is typically a replication-defective retro virus carrying the transgene (Jahner et al., Proc. Natl. Acad. Sci. USA 82:6927-6931, 1985; Van der Putten et al., Proc. Natl. Acad. Sci USA 82:6148-6152, 1985). Transfection is easily and efficiently obtained by culturing the blastomeres on a monolayer of virus-producing cells (Van der Putten, supra; Stewart et al., EMBO J. 6:383-388, 1987). Alternatively, infection can be performed at a later stage. Virus or virus-producing cells can be injected into the blastocoele (Jahner et al., Nature 298:623-628, 1982). Most of the founders will be mosaic for the transgene since incorporation occurs only in a subset of the cells which formed the transgenic nonhuman animal. Further, the founder may contain various retro viral insertions of the transgene at different positions in the genome which generally will segregate in the offspring. In addition, it is also possible to introduce transgenes into the germ line, albeit with low efficiency, by intrauterine retroviral infection of the midgestation embryo (D. Jahner et al., supra).

A third type of target cell for transgene introduction is the embryonal stem cell (ES). ES cells are obtained from pre-implantation embryos cultured in vitro and fused with embryos (Evans et al., Nature 292:154-156, 1981; Bradley et al., Nature 309: 255-258, 1984; Gossler et al., Proc. Natl. Acad. Sci USA 83: 9065-9069, 1986; and Robertson et al., Nature 322:445-448, 1986). Transgenes can be efficiently introduced into the ES cells by DNA transfection or by retrovirus-mediated transduction. Such transformed ES cells can thereafter be combined with blastocysts from a nonhuman animal. The ES cells thereafter colonize the embryo and contribute to the germ line of the resulting chimeric animal. (For review see Jaenisch, Science 240: 1468-1474, 1988).

“Transformed” means a cell into which (or into an ancestor of which) has been introduced, by means of recombinant nucleic acid techniques, a heterologous nucleic acid molecule. “Heterologous” refers to a nucleic acid sequence that either originates from another species or is modified from either its original form or the form primarily expressed in the cell.

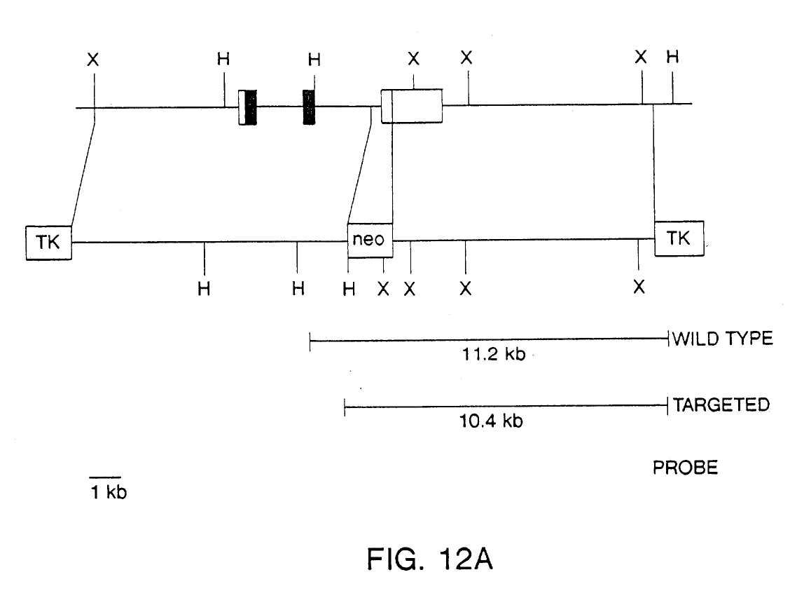

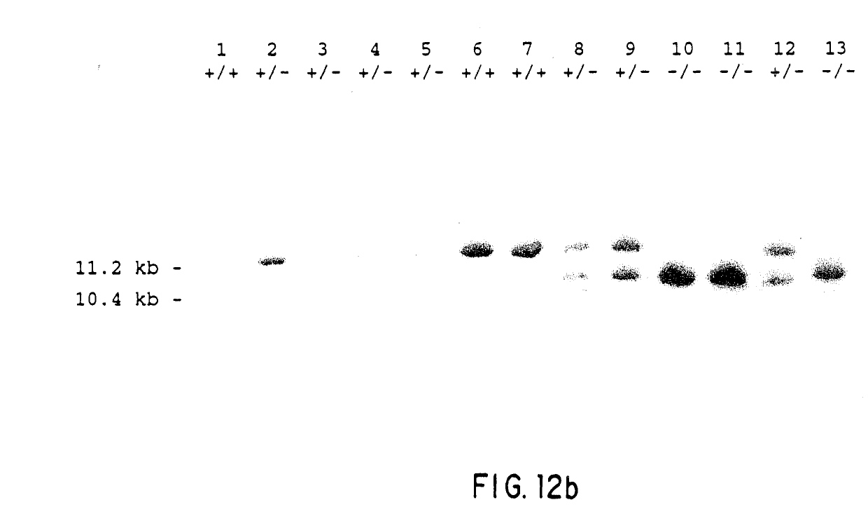

“Transgene” means any piece of DNA which is inserted by artifice into a cell, and becomes part of the genome of the organism (i.e., either stably integrated or as a stable extrachromosomal element) which develops from that cell. Such a transgene may include a gene which is partly or entirely heterologous (i.e., foreign) to the transgenic organism, or may represent a gene homologous to an endogenous gene of the organism. Included within this definition is a transgene created by the providing of an RNA sequence which is transcribed into DNA and then incorporated into the genome. The transgenes of the invention include DNA sequences which encode GDF-8, and include GDF-sense, antisense, dominant negative encoding polynucleotides, which may be expressed in a transgenic non-human animal. The term “transgenic” as used herein additionally includes any organism whose genome has been altered by in vitro manipulation of the early embryo or fertilized egg or by any transgenic technology to induce a specific gene knockout. The term “gene knockout” as used herein, refers to the targeted disruption of a gene in vivo with complete loss of function that has been achieved by any transgenic technology familiar to those in the art. In one embodiment, transgenic animals having gene knockouts are those in which the target gene has been rendered nonfunctional by an insertion targeted to the gene to be rendered non-functional by homologous recombination. As used herein, the term “transgenic” includes any transgenic technology familiar to those in the art which can produce an organism carrying an introduced transgene or one in which an endogenous gene has been rendered non-functional or “knocked out.” An example of a transgene used to “knockout” GDF-8 function in the present Examples is described in Example 8 and FIG. 12A. Thus, in another embodiment, the invention provides a transgene wherein the entire mature C-terminal region of GDF-8 is deleted.

The transgene to be used in the practice of the subject invention is a DNA sequence comprising a modified GDF-8 coding sequence. In a preferred embodiment, the GDF-8 gene is disrupted by homologous targeting in embryonic stem cells. For example, the entire mature C-terminal region of the GDF-8 gene may be deleted as described in the examples below. Optionally, the GDF-8 disruption or deletion may be accompanied by insertion of or replacement with other DNA sequences, such as a non-functional GDF-8 sequence. In other embodiments, the transgene comprises DNA antisense to the coding sequence for GDF-8. In another embodiment, the transgene comprises DNA encoding an antibody or receptor peptide sequence which is able to bind to GDF-8. The DNA and peptide sequences of GDF-8 are known in the art, the sequences, localization and activity disclosed in WO 94/21681, incorporated by reference in its entirety. The disclosure of both of these applications are hereby incorporated herein by reference. Where appropriate, DNA sequences that encode proteins having GDF-8 activity but differ in nucleic acid sequence due to the degeneracy of the genetic code may also be used herein, as may truncated forms, allelic variants and interspecies homologues.

The invention also includes animals having heterozygous mutations in GDF-8 or partial inhibition of GDF-8 function or expression. A heterozygote would exhibit an intermediate increase in muscle and/or bone mass as compared to the homozygote as shown in Table 4 below. In other words, partial loss of function leads to a partial increase in muscle and bone mass. One of skill in the art would readily be able to determine if a particular mutation or if an antisense molecule was able to partially inhibit GDF-8. For example, in vitro testing may be desirable initially by comparison with wild-type or untreated GDF-8 (e.g., comparison of northern blots to examine a decrease in expression).

After an embryo has been microinjected, colonized with transfected embryonic stem cells or infected with a retrovirus containing the transgene (except for practice of the subject invention in avian species which is addressed elsewhere herein) the embryo is implanted into the oviduct of a pseudopregnant female. The consequent progeny are tested for incorporation of the transgene by Southern blot analysis of blood samples using transgene specific probes. PCR is particularly useful in this regard. Positive progeny (G0) are crossbred to produce offspring (G1) which are analyzed for transgene expression by northern blot analysis of tissue samples. To be able to distinguish expression of like-species transgenes from expression of the animals endogenous GDF-8 gene(s), a marker gene fragment can be included in the construct in the 3′ untranslated region of the transgene and the northern probe designed to probe for the marker gene fragment. The serum levels of GDF-8 can also be measured in the transgenic animal to establish appropriate expression. Expression of the GDF-8 transgenes, thereby decreasing the GDF-8 in the tissue and serum levels of the transgenic animals and consequently increasing the muscle tissue or bone tissue content results in the foodstuffs from these animals (i.e., eggs, beef, pork, poultry meat, milk, etc.) having markedly increased muscle and/or bone content, such as ribs, and preferably without increased, and more preferably, reduced levels of fat and cholesterol. By practice of the subject invention, a statistically significant increase in muscle content, preferably at least a 2% increase in muscle content (e.g., in chickens), more preferably a 25% increase in muscle content as a percentage of body weight, more preferably greater than 40% increase in muscle content in these foodstuffs can be obtained. Similarly the subject invention may provide a significant increase in bone content, such as ribs, in these foodstuffs.

Additional Methods of Use

Thus, the present invention includes methods for increasing muscle and bone mass in domesticated animals, characterized by inactivation or deletion of the gene encoding growth and differentiation factor-8 (GDF-8). The domesticated animal is preferably selected from the group consisting of ovine, bovine, porcine, piscine and avian. The animal may be treated with an isolated polynucleotide sequence encoding growth and differentiation factor-8 which polynucleotide sequence is also from a domesticated animal selected from the group consisting of ovine, bovine, porcine, piscine and avian. The present invention includes methods for increasing the muscle and/or bone mass in domesticated animals characterized by administering to a domesticated animal monoclonal antibodies directed to the GDF-8 polypeptide. The antibody may be an anti-GDF-8, and may be either a monoclonal antibody or a polyclonal antibody.

The invention includes methods comprising using an anti-GDF-8 monoclonal antibody, antisense, or dominant negative mutants as a therapeutic agent to inhibit the growth regulating actions of GDF-8 on muscle and bone cells. Muscle and bone cells are defined to include fetal or adult muscle cells, as well as progenitor cells which are capable of differentiation into muscle or bone. The monoclonal antibody may be a humanized (e.g., either fully or a chimeric) monoclonal antibody, of any species origin, such as murine, ovine, bovine, porcine or avian. Methods of producing antibody molecules with various combinations of “humanized” antibodies are well known in the art and include combining murine variable regions with human constant regions (Cabily et al. Proc. Natl. Acad. Sci. USA, 81:3273, 1984), or by grafting the murine-antibody complementary determining regions (CDRs) onto the human framework (Richmann et al., Nature 332:323, 1988). Other general references which teach methods for creating humanized antibodies include Morrison et al., Science, 229:1202, 1985; Jones et al., Nature, 321:522, 1986; Monroe et al., Nature 312:779, 1985; Oi et al., BioTechniques, 4:214, 1986; European Patent Application No. 302,620; and U.S. Pat. No. 5,024,834. Therefore, by humanizing the monoclonal antibodies of the invention for in vivo use, an immune response to the antibodies would be greatly reduced.

The monoclonal antibody, GDF-8 polypeptide, or GDF-8 polynucleotide (all “GDF-8 agents”) may have the effect of increasing the development of skeletal muscles and bones, such as ribs. In preferred embodiments of the claimed methods, the GDF-8 monoclonal antibody, polypeptide, or polynucleotide is administered to a patient suffering from a disorder selected from the group consisting of muscle wasting disease, neuromuscular disorder, muscle atrophy, bone degenerative diseases, osteoporosis, renal disease or aging. The GDF-8 agent may also be administered to a patient suffering from a disorder selected from the group consisting of muscular dystrophy, spinal cord injury, traumatic injury, congestive obstructive pulmonary disease (COPD), AIDS or cachexia. In a preferred embodiment, the GDF-8 agent is administered to a patient suffering from any of these diseases by intravenous, intramuscular or subcutaneous injection; preferably, a monoclonal antibody is administered within a dose range between about 0.1 mg/kg to about 100 mg/kg; more preferably between about 1 μg/kg to 75 mg/kg; most preferably from about 10 mg/kg to 50 mg/kg. The antibody may be administered, for example, by bolus injunction or by slow infusion. Slow infusion over a period of 30 minutes to 2 hours is preferred. The GDF-8 agent may be formulated in a formulation suitable for administration to a patient. Such formulations are known in the art.

The dosage regimen will be determined by the attending physician considering various factors which modify the action of the GDF-8 protein, e.g., amount of tissue desired to be formed, the site of tissue damage, the condition of the damaged tissue, the size of a wound, type of damaged tissue, the patient's age, sex, and diet, the severity of any infection, time of administration and other clinical factors. The dosage may vary with the type of matrix used in the reconstitution and the types of agent, such as anti-GDF-8 antibodies, to be used in the composition. Generally, systemic or injectable administration, such as intravenous (IV), intramuscular (IM) or subcutaneous (Sub-Q) injection. Administration will generally be initiated at a dose which is minimally effective, and the dose will be increased over a preselected time course until a positive effect is observed. Subsequently, incremental increases in dosage will be made limiting such incremental increases to such levels that produce a corresponding increase in effect, while taking into account any adverse affects that may appear. The addition of other known growth factors, such as IGF I (insulin like growth factor I), human, bovine, or chicken growth hormone which may aid in increasing muscle and bone mass, to the final composition, may also affect the dosage. In the embodiment where an anti-GDF-8 antibody is administered, the anti-GDF-8 antibody is generally administered within a dose range of about 0.1 μg/kg to about 100 mg/kg.; more preferably between about 10 mg/kg to 50 mg/kg.

Progress can be monitored by periodic assessment of tissue growth and/or repair. The progress can be monitored, for example, X-rays, histomorphometric determinations and tetracycline labeling.

Screening for GDF-8 Modulating Compounds

In another embodiment, the invention provides a method for identifying a compound or molecule that modulates GDF-8 protein activity or gene expression. The method includes incubating components comprising the compound, GDF-8 polypeptide or with a recombinant cell expressing GDF-8 polypeptide, under conditions sufficient to allow the components to interact and determining the effect of the compound on GDF-8 activity or expression. The effect of the compound on GDF-8 activity can be measured by a number of assays, and may include measurements before and after incubating in the presence of the compound. Compounds that affect GDF-8 activity or gene expression include peptides, peptidomimetics, polypeptides, chemical compounds and biologic agents. Assays include northern blot analysis of GDF-8 MRNA (for gene expression), western blot analysis (for protein level) and muscle fiber analysis (for protein activity).

The above screening assays may be used for detecting the compounds or molecules that bind to the GDF-8 receptor or GDF-8 polypeptide, in isolating molecules that bind to the GDF-8 gene, for measuring the amount of GDF-8 in a sample, either polypeptide or RNA (mRNA), for identifying molecules that may act as agonists or antagonists, and the like. For example, GDF-8 antagonists are useful for treatment of muscular and adipose tissue disorders (e.g., obesity).

Incubating includes conditions which allow contact between the test compound and GDF-8 polypeptide or with a recombinant cell expressing GDF-8 polypeptide. Contacting includes in solution and in solid phase, or in a cell. The test compound may optionally be a combinatorial library for screening a plurality of compounds. Compounds identified in the method of the invention can be further evaluated, detected, cloned, sequenced, and the like, either in solution or after binding to a solid support, by any method usually applied to the detection of a specific DNA sequence such as PCR, oligomer restriction (Saiki et al., BioTechnology, 3:1008-1012, 1985), allele-specific oligonucleotide (ASO) probe analysis (Conner et al., Proc. Natl. Acad. Sci. USA, 80:278, 1983), oligonucleotide Landegren et al., Science, 241:1077, 1988), and the like. Molecular techniques for DNA analysis have been reviewed (Landegren et al., Science, 242:229-237, 1988).

All references cited herein are hereby incorporated by reference in their entirety.

The following examples are intended to illustrate but not limit the invention. While they are typical of those that might be used, other procedures known to those skilled in the art may alternatively be used.

### EXAMPLE 1

Identification and Isolation of a Novel TGF-β Family Member

To identify a new member of the TGF-β superfamily, degenerate oligonucleotides were designed which corresponded to two conserved regions among the known family members: one region spanning the two tryptophan residues conserved in all family members except MIS and the other region spanning the invariant cysteine residues near the C-terminus. These primers were used for polymerase chain reactions on mouse genomic DNA followed by subcloning the PCR products using restriction sites placed at the 5′ ends of the primers, picking individual E. coli colonies carrying these subcloned inserts, and using a combination of random sequencing and hybridization analysis to eliminate known members of the superfamily.

GDF-8 was identified from a mixture of PCR products obtained with the primers SJL141:

| | |
|---|---|
| 5′-CCGGAATTCGGITGG(G/C/A)A(G/A/T/C)(A/G)A(T/C)TGG(A/G)TI(A/G)TI(T/G)CICC-3′; | (SEQ ID NO:1) |
| SJL147: 5′-CCGGAATTC(G/A)CAI(G/C)C(G/A)CA(G/A)CT(GIA/T/C)TCIACI(G/A)(T/C)CAT-3′. | (SEQ ID NO:2) |

PCR using these primers was carried out with 2 μg mouse genomic DNA at 94° C. for 1 min, 50° C. for 2 min, and 72° C. for 2 min for 40 cycles.

PCR products of approximately 280 bp were gel-purified, digested with EcoRI, gel-purified again, and subcloned in the BLUESCRIPT vector (Stratagene, San Diego, Calif.). Bacterial colonies carrying individual subclones were picked into 96 well microtiter plates, and multiple replicas were prepared by plating the cells onto nitrocellulose. The replicate filters were hybridized to probes representing known members of the family, and DNA was prepared from nonhybridizing colonies for sequence analysis.

The primer combination of SJL141 and SJL147, encoding the amino acid sequences GW(H/Q/N/K/D/E)(DIN)W(V/I/M)(V/I/M)(A/S)P (SEQ ID NO: 9) and M(V/I/M/T/A)V(D/E)SC(G/A)C (SEQ ID NO: 10), respectively, yielded four previously identified sequences (BMP-4, inhibin βB, GDF-3 and GDF-5) and one novel sequence, which was designated GDF-8, among 110 subclones analyzed.

Human GDF-8 was isolated using the primers:

(SEQ ID NO:3)

ACM13: 5′-CGCGGATCCAGAGTCAAGGTGACAGACACAC-3′;
and

(SEQ ID NO:4)

ACM14: 5′-CGCGGATCCTCCTCATGAGCACCCACAGCGGTC-3′.

PCR using these primers was carried out with one pg human genomic DNA at 94° C. for 1 min, 58° C. for 2 min, and 72° C. for 2 min for 30 cycles. The PCR produc digested with Bam HI, gel-purified, and subcloned in the BLUESCRIPT vector (Stratagene).

### EXAMPLE 2

Expression Pattern and Sequence of GDF-8

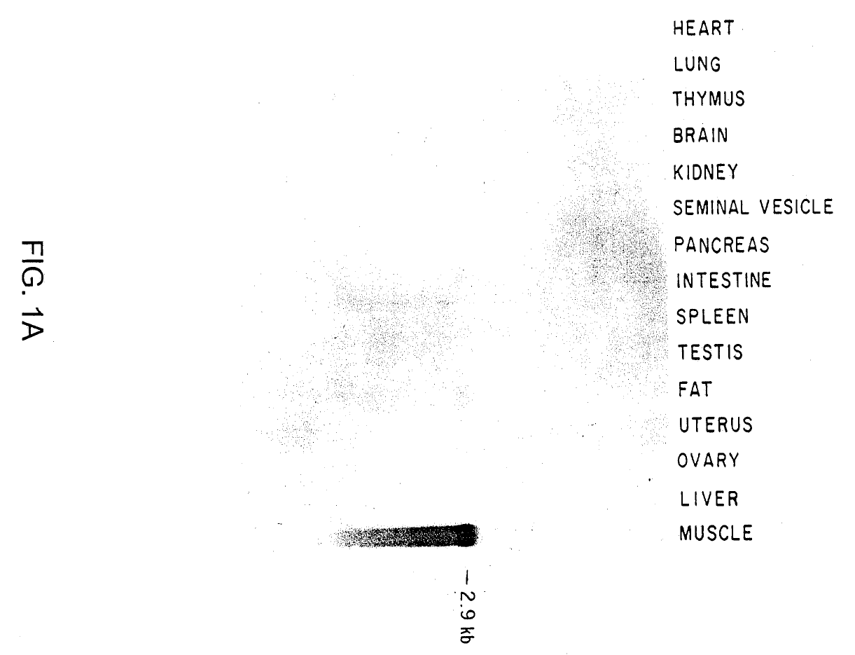

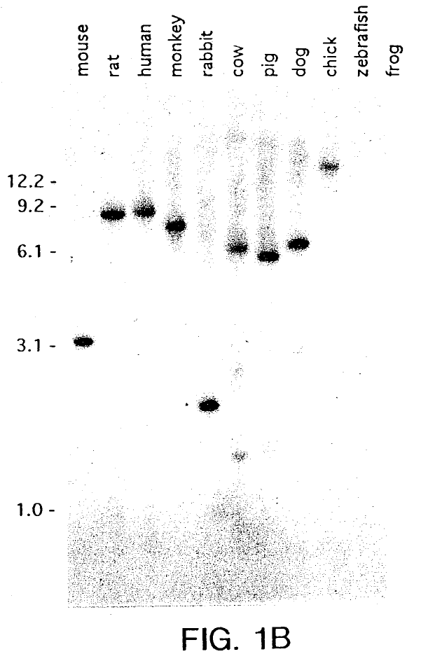

To determine the expression pattern of GDF-8, RNA samples prepared from a variety of adult tissues were screened by Northern analysis. RNA isolation and northern blot analysis were carried out as described previously (Lee., Mol. Endocrinol., 4:1034, 1990) except that hybridization was carried out in 5×SSPE, 10% dextran sulfate, 50% formamide, 1% SDS, 200 Ag/ml salmon DNA, and 0.1% each of bovine serum albumin, ficoll, and polyvinylpyrrolidone. Five micrograms of twice poly A-selected RNA prepared from each tissue (except for muscle, for which only 2 μg RNA was used) were electrophoresed on formaldehyde gels, blotted, and probed with GDF-8. As shown in FIG. 1, the GDF-8 probe detected a single mRNA species expressed at highest levels in muscle and at significantly lower levels in adipose tissue.

To obtain a larger segment of the GDF-8 gene, a mouse genomic library was screened with a probe derived from the GDF-8 PCR product. The partial sequence of a GDF-8 genomic clone is shown in FIG. 2A (SEQ ID NO: 5). The sequence contains an open reading frame corresponding to the predicted C-terminal region of the GDF-8 precursor protein. The predicted GDF-8 sequence contains two potential proteolytic processing sites, which are boxed. Cleavage of the precursor at the second of these sites would generate a mature C-terminal fragment 109 amino acids in length with a predicted molecular weight of 12,400 Da. The partial sequence of human GDF-8 is shown in FIG. 2B (SEQ ID NO: 7). Assuming no PCR-induced errors during the isolation of the human clone, the human and mouse amino acid sequences in this region are 100% identical.

The C-terminal region of GDF-8 following the putative proteolytic processing site shows significant homology to the known members of the TGF-β; superfamily (FIG. 3; SEQ ID NOS: 36 to 49). FIG. 3 shows the alignment of the C-terminal sequences of GDF-8 with the corresponding regions of human GDF-1 (Lee, Proc. Natl. Acad. Sci. USA, 88:4250-4254, 1991), human BMP-2 and 4 (Wozney et al., Science, 242:1528-1534, 1988), human Vgr-1 (Celeste et al., Proc. Natl. Acad. Sci. USA, 87:9843-9847, 1990), human OP-1 (Ozkaynak et al., EMBO J., 9:2085-2093, 1990), human BMP-5 (Celeste et al., Proc. Natl. Acad. Sci. USA, 87:9843-9847, 1990), human BMP-3 (Wozney et al., Science, 242:1528-1534, 1988), human MiS (Cate et al., Cell, 45:685-698,1986), human inhibin alpha, βA, and βB (Mason et al., Biochem, Biophys. Res. Comm., 135:957-964, 1986), human TGF-β1 (Derynek et al., Nature, 316:701-705, 1985), human TGF-β2 (deMartin et al., EMBO J., 6:3673-3677, 1987), and human TGF-β3 (ten Dijke et al., Proc. Natl. Acad. Sci. USA, 85:4715-4719, 1988). The conserved cysteine residues are boxed. Dashes denote gaps introduced in order to maximize the alignment.

GDF-8 contains most of the residues that are highly conserved in other family members, including the seven cysteine residues with their characteristic spacing. Like the TGF-βs and inhibin βs, GDF-8 also contains two additional cysteine residues. In the case of TGF-β2, these two additional cysteine residues are known to form an intramolecular disulfide bond (Daopin et al., Science, 257:369, 1992; Schlunegger and Grutter, Nature, 358:430, 1992).

Figure shows the amino acid homologies among the different members of the TGF-β superfamily. Numbers represent percent amino acid identities between each pair calculated from the first conserved cysteine to the C terminus. Boxes represent homologies among highly-related members within particular subgroups. In this region, GDF-8 is most homologous to Vgr-1 (45% sequence identity).

### EXAMPLE 3

Isolation of cDNA Clones Encoding Murine and Human GDF-8

In order to isolate full-length cDNA clones encoding murine and human GDF-8, cDNA libraries were prepared in the lambda ZAP II vector (Stratagene) using RNA prepared from skeletal muscle. From 5 μg of twice poly A-selected RNA prepared from murine and human muscle, cDNA libraries consisting of 4.4 million and 1.9 million recombinant phage, respectively, were constructed according to the instructions provided by Stratagene. These libraries were screened without amplification. Library screening and characterization of cDNA inserts were carried out as described previously (Lee, Mol. Endocrinol., 4:1034-1040).

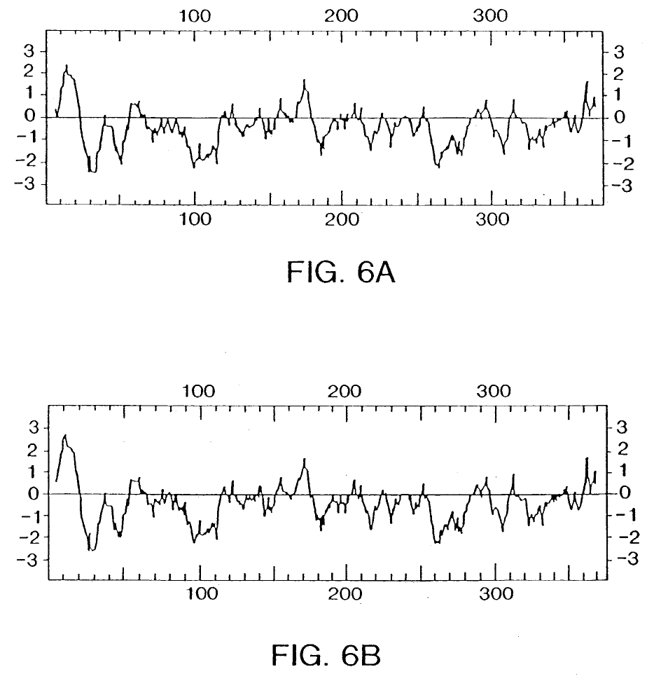

From 2.4×10⁶ recombinant phage screened from the murine muscle cDNA library, greater than 280 positive phage were identified using a murine GDF-8 probe derived from a genomic clone, as described in Example 1. The entire nucleotide sequence of the longest cDNA insert analyzed is shown in FIGS. 5A and 5B and SEQ ID NO: 11. The 2676 base pair sequence contains a single long open reading frame beginning with a methionine codon at nucleotide 104 and extending to a TGA stop codon at nucleotide 1232. Upstream of the putative initiating methionine codon is an in-frame stop codon at nucleotide 23. The predicted pre-pro-GDF-8 protein is 76 amino acids in length. The sequence contains a core of hydrophobic amino acids at the N-terminus suggestive of a signal peptide for secretion (FIG. 6A), one potential N-glycosylation site at asparagine 72, a putative RXXR (SEQ ID NO: 50) proteolytic cleavage site at amino acids 264-267, and a C-terminal region showing significant homology to the known members of the TGF-β superfamily. Cleavage of the precursor protein at the putative RXXR (SEQ ID NO: 50) site would generate a mature C-terminal GDF-8 fragment 109 amino acids in length with a predicted molecular weight of approximately 12,400 Da.

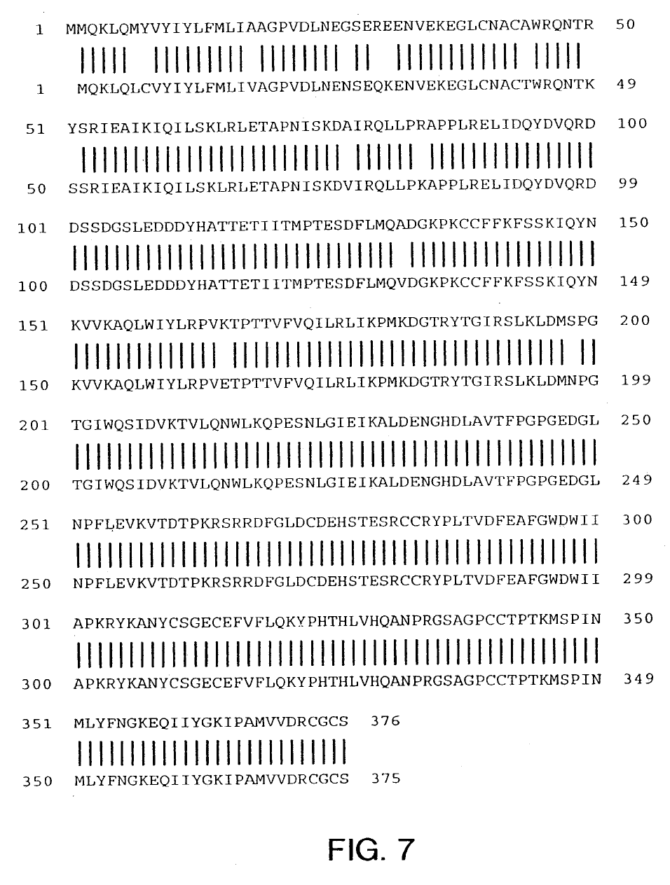

From 1.9×10⁶ recombinant phage screened from the human muscle cDNA library, 4 positive phage were identified using a human GDF-8 probe derived by polymerase chain reaction on human genomic DNA. The entire nucleotide sequence of the longest cDNA insert is shown in FIGS. 5C and 5D and SEQ ID NO: 13. The 2743 base pair sequence contains a single long open reading frame beginning with a methionine codon at nucleotide 59 and extending to a TGA stop codon at nucleotide 1184. The predicted pre-pro-GDF-8 protein is 375 amino acids in length. The sequence contains a core of hydrophobic amino acids at the N-terminus suggestive of a signal peptide for secretion (FIG. 6B), one potential N-glycosylation site at asparagine 71, and a putative RXXR (SEQ ID NO: 50) proteolytic cleavage site at amino acids 263-266. FIG. 7 shows a comparison of the predicted murine (top) and human (bottom) GDF-8 amino acid sequences. Numbers indicate amino acid position relative to the N-terminus. Identities between the two sequences are denoted by a vertical line. Murine and human GDF-8 are approximately 94% identical in the predicted pro-regions and 100% identical following the predicted RXXR (SEQ ID NO: 50) cleavage sites.

### EXAMPLE 4

Dimerization of GDF-8

To determine whether the processing signals in the GDF-8 sequence are functional and whether GDF-8 forms dimers like other members of the TGF-β superfamily, the GDF-8 cDNA was stably expressed in CHO cells. The GDF-8 coding sequence was cloned into the pMSXND expression vector (Lee and Nathans, J. Biol. Chem., 263:3521, 1988) and transfected into CHO cells. Following G418 selection, the cells were selected in 0.2 μM methotrexate, and conditioned medium from resistant cells was concentrated and electrophoresed on SDS gels. Conditioned medium was prepared by Cell Trends, Inc. (Middletown, Md.). For preparation of anti-GDF-8 serum, the C-terminal region of GDF-8 (amino acids 268 to 376) was expressed in bacteria using the RSET vector (Invitrogen Corp., San Diego, Calif.), purified using a nickel chelate column, and injected into rabbits. All immunizations were carried out by Spring Valley Labs (Woodbine, Md.). Western blot analysis using (¹²⁵I)-iodoprotein-A was carried out as described (Burnette, Anal. Biochem., 112:195, 1981). Western blot analysis of conditioned medium prepared from these cells using an antiserum raised against a bacterially expressed C-terminal fragment of GDF-8 detected two protein species with apparent molecular weights of approximately 52 kDa and 15 kDa under reducing conditions, consistent with unprocessed and processed forms of GDF-8, respectively. No bands were obtained either with preimmune serum or with conditioned medium from CHO cells transfected with an antisense construct. Under non-reducing conditions, the GDF-8 antiserum detected two predominant protein species with apparent molecular weights of approximately 101 kDa and 25 kDa, consistent with dimeric forms of unprocessed and processed GDF-8, respectively. Hence, like other TGF-β family members, GDF-8 appears to be secreted and proteolytically processed, and the C-terminal region appears to be capable of forming a disulfide-linked dimer.

### EXAMPLE 5

Preparation of Antibodies Against GDF-8 and Expression of GDF-8 in Mammalian Cells

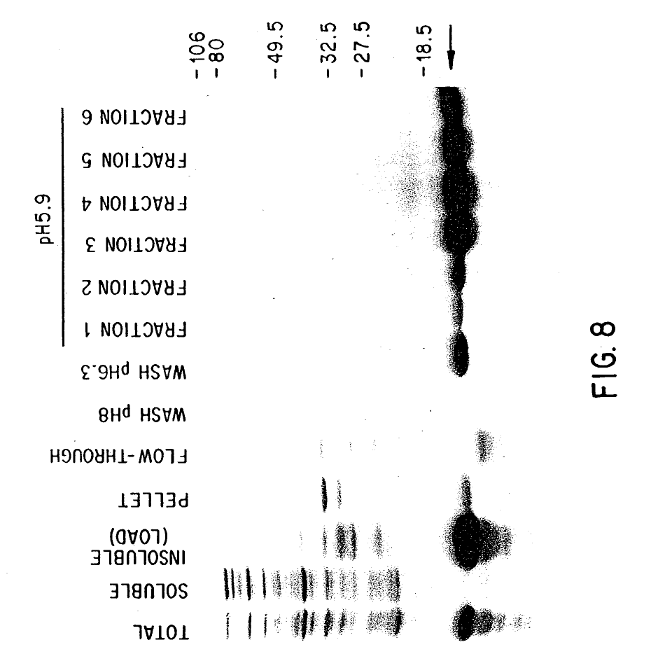

In order to prepare antibodies against GDF-8, GDF-8 antigen was expressed as a fusion protein in bacteria. A portion of murine GDF-8 cDNA spanning amino acids 268-376 (mature region) was inserted into the pRSET vector (Invitrogen) such that the GDF-8 coding sequence was placed in frame with the initiating methionine codon present in the vector; the resulting construct created an open reading frame encoding a fusion protein with a molecular weight of approximately 16,600 kDa. The fusion construct was transformed into BL21 (DE3) (pLysS) cells, and expression of the fusion protein was induced by treatment with isopropylthio-β-galactoside as described (Rosenberg et al., Gene, 56:125-135). The fusion protein was then purified by metal chelate chromatography according to the instructions provided by Invitrogen. A Coomassie blue-stained gel of unpurified and purified fusion proteins is shown in FIG. 8.

The purified fusion protein was used to immunize both rabbits and chickens. Immunization of rabbits was carried out by Spring Valley Labs (Sykesville, Md.), and immunization of chickens was carried out by HRP, Inc. (Denver, Pa.). Western blot analysis of sera both from immunized rabbits and from immunized chickens demonstrated the presence of antibodies directed against the fusion protein.

To express GDF-8 in mammalian cells, the murine GDF-8 cDNA sequence from nucleotides 48-1303 was cloned in both orientations downstream of the metallothionein I promoter in the pMSXND expression vector; this vector contains processing signals derived from SV40, a dihydrofolate reductase gene, and a gene conferring resistance to the antibiotic G418 (Lee and Nathans, J. Biol. Chem., 263:3521-3527). The resulting constructs were transfected into Chinese hamster ovary cells, and stable transfectants were selected in the presence of G418. Two milliliters of conditioned media prepared from the G418-resistant cells were dialyzed, lyophilized, electrophoresed under denaturing, reducing conditions, transferred to nitrocellulose, and incubated with anti-GDF-8 antibodies (described above) and (¹²⁵I)-iodoprotein-A.

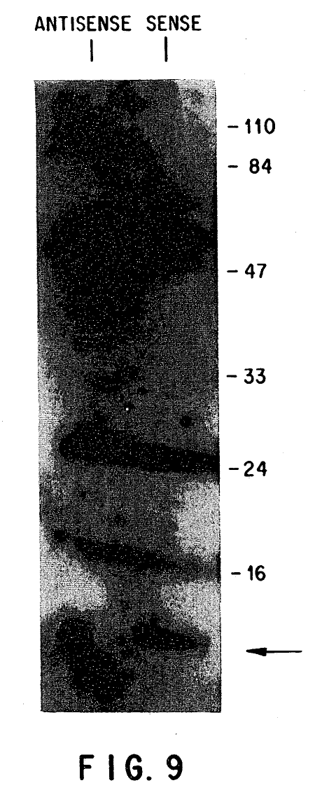

As shown in FIG. 9, the rabbit GDF-8 antibodies (at a 1:500 dilution) detected a protein of approximately the predicted molecular weight for the mature C-terminal fragment of GDF-8 in the conditioned media of cells transfected with a construct in which GDF-8 had been cloned in the correct (sense) orientation with respect to the metallothionein promoter (lane 2); this band was not detected in a similar sample prepared from cells transfected with a control antisense construct (lane 1). Similar results were obtained using antibodies prepared in chickens. Hence, GDF-8 is secreted and proteolytically processed by these transfected mammalian cells.

### EXAMPLE 6

Expression Pattern of GDF-8

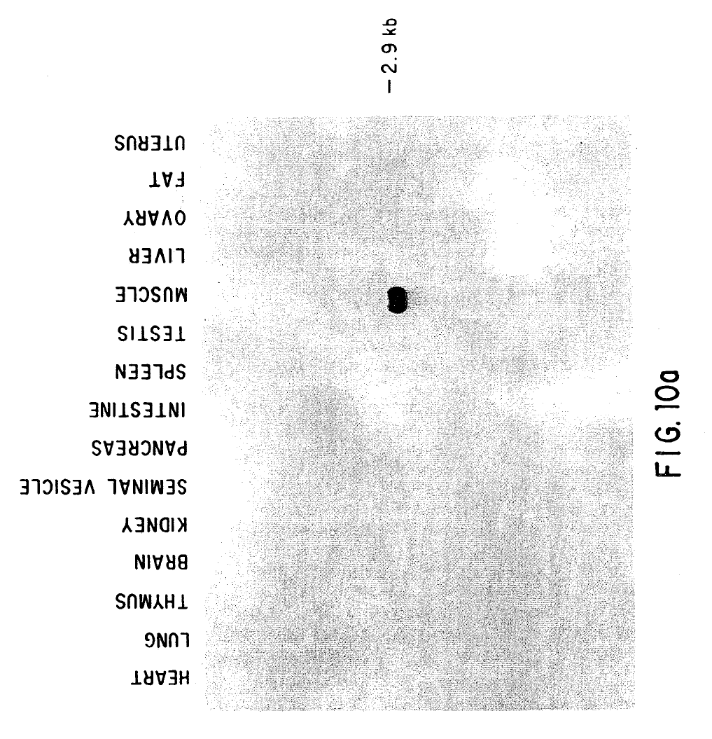

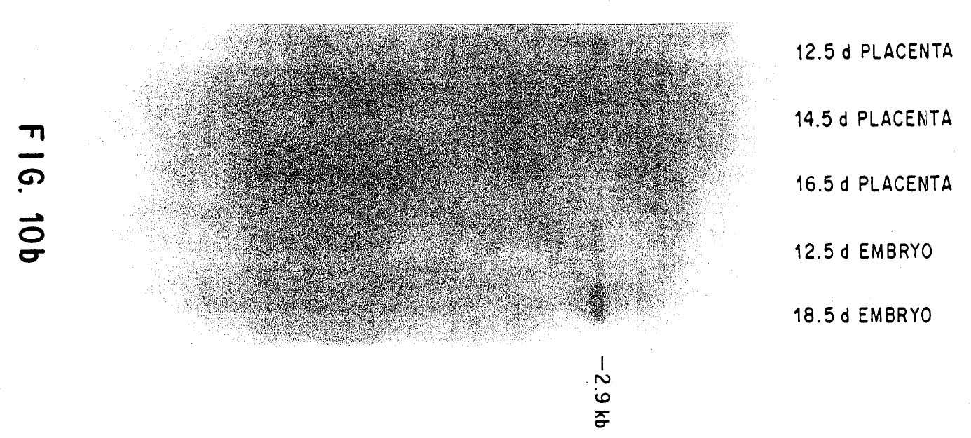

To determine the pattern of GDF-8, 5 μg of twice poly A-selected RNA prepared from a variety of murine tissue sources were subjected to northern blot analysis. As shown in FIG. 10A (and as shown previously in Example 2), the GDF-8 probe detected a single mRNA species present almost exclusively in skeletal muscle among a large number of adult tissues surveyed. On longer exposures of the same blot, significantly lower but detectable levels of GDF-8 mRNA were seen in fat, brain, thymus, heart, and lung. Hence, these results confirm the high degree of specificity of GDF-8 expression in skeletal muscle. GDF-8 mRNA was also detected in mouse embryos at both gestational ages (day 12.5 and day 18.5 post-coital) examined but not in placentas at various stages of development (FIG. 10B).

To further analyze the expression pattern of GDF-8, in situ hybridization was performed on mouse embryos isolated at various stages of development. For all in situ hybridization experiments, probes corresponding to the C-terminal region of GDF-8 were excluded in order to avoid possible cross-reactivity with other members of the superfamily. Whole mount in situ hybridization analysis was carried out as described (Wilkinson, In Situ Hybridization, A Practical Approach, pp. 75-83, IRL. Press, Oxford, 1992) except that blocking and antibody incubation steps were carried out as in Knecht et al. (Development, 121:1927, 1955). Alkaline phosphatase reactions were carried out for 3 hours for day 10.5 embryos and overnight for day 9.5 embryos. Hybridization was carried out using digoxigenin-labeled probes spanning nucleotides 8-811 and 1298-2676, which correspond to the pro-region and 3′ untranslated regions, respectively. In situ hybridization to sections was carried out as described (Wilkinson et al., Cell, 50:79, 1987) using ³⁵S-labeled probes ranging from approximately 100-650 bases in length and spanning nucleotides 8-793 and 1566-2595. Following hybridization and washing, slides were dipped in NTB-3 photographic emulsion, exposed for 16-19 days, developed and stained with either hematoxylin and eosin or toluidine blue. RNA isolation, poly A selection, and Northern analysis were carried out as described previously (McPherron and Lee, J. Biol. Chem., 268:3444, 1993).

At all stages examined, the expression of GDF-8 mRNA appeared to be restricted to developing skeletal muscle. At early stages, GDF-8 expression was restricted to developing somites. By whole mount in situ hybridization analysis, GDF-8 mRNA could first be detected as early as day 9.5 post coitum in approximately one-third of the somites. At this stage of development, hybridization appeared to be restricted to the most mature (9 out of 21 in this example), rostral somites. By day 10.5 p.c., GDF-8 expression was clearly evident in almost every somite (28 out of 33 in this example shown). Based on in situ hybridization analysis of sections prepared from day 10.5 p.c. embryos, the expression of GDF-8 in somites appeared to be localized to the myotome compartment. At later stages of development, GDF-8 expression was detected in a wide range of developing muscles.

GDF-8 continues to be expressed in adult animals as well. By northern blot analysis, GDF-8 mRNA expression was seen almost exclusively in skeletal muscle among the different adult tissues examined. A significantly lower though clearly detectable signal was also seen in adipose tissue. Based on northern blot analysis of RNA prepared from a large number of different adult skeletal muscles, GDF-8 expression appeared to be widespread although the expression levels varied among individual muscles.

### EXAMPLE 7

Chromosomal Localization of GDF-8

In order to map the chromosomal location of GDF-8, DNA samples from human/rodent somatic cell hybrids (Drwinga et al., Genomics, 16:311-413, 1993; Dubois and Naylor, Genomics, 16:315-319, 1993) were analyzed by polymerase chain reaction followed by Southern blotting. Polymerase chain reaction was carried out using primer #83, 5′-CGCGGATCCGTGGATCTAAATGAGAACAGTGAGC-³ (SEQ ID NO: 15) and primer #84, 5′-CGCGAATTCTCAGGTAATGATTGTTTCCGTTGTAGCG-³′ (SEQ ID NO: 16) for 40 cycles at 94° C. for 2 minutes, 60° C. for 1 minute, and 72° C. for 2 minutes. These primers correspond to nucleotides 119 to 143 (flanked by a Bam HI recognition sequence), and nucleotides 394 to 418 (flanked by an Eco RI recognition sequence), respectively, in the human GDF-8 cDNA sequence. PCR products were electrophoresed on agarose gels, blotted, and probed with oligonucleotide #100, 5′-ACACTAAATCTTCAAGAATA-3′ (SEQ ID NO: 17), which corresponds to a sequence internal to the region flanked by primer #83 and #84. Filters were hybridized in 6×SSC, 1× Denhardt's solution, 100 μg/ml yeast transfer RNA, and 0.05% sodium pyrophosphate at 50° C.

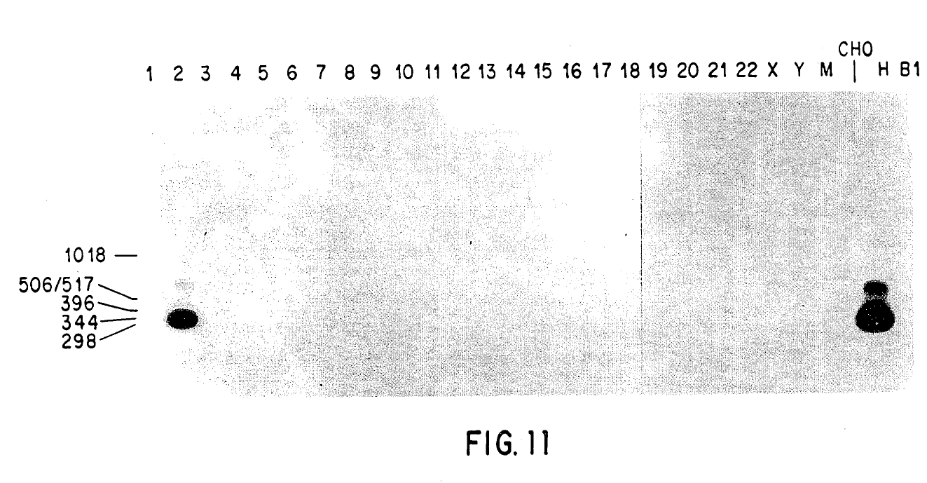

As shown in FIG. 11, the human-specific probe detected a band of the predicted size (approximately 320 base pairs) in the positive control sample (total human genomic DNA) and in a single DNA sample from the human/rodent hybrid panel. This positive signal corresponds to human chromosome 2. The human chromosome contained in each of the hybrid cell lines is identified at the top of each of the first 24 lanes (1-22, X, and Y). In the lanes designated M, CHO, and H, the starting DNA template was total genomic DNA from mouse, hamster, and human sources, respectively. In the lane marked B1, no template DNA was used. Numbers at left indicate the mobilities of DNA standards. These data show that the human GDF-8 gene is located on chromosome 2.

### EXAMPLE 8

GDF-8 Transgenic Knockout Mice

The GDF-8, we disrupted the GDF-8 gene was disrupted by homologous targeting in embryonic stem cells. To ensure that the resulting mice would be null for GDF-8 function, the entire mature C-terminal region was deleted and replaced by a neo cassette (FIG. 12A). A murine 129 SV/J genomic library was prepared in lambda FIX II according to the instructions provided by Stratagene. The structure of the GDF-8 gene was deduced from restriction mapping and partial sequencing of phage clones isolated from this library. Vectors for preparing the targeting construct were kindly provided by Philip Soriano and Kirk Thomas University. R1 ES cells were transfected with the targeting construct, selected with gancyclovir (2 μM) and G418 (250 μg/ml), and analyzed by Southern blot analysis. Homologously targeted clones were injected into C57BL/6 blastocysts and transferred into pseudopregnant females. Germline transmission of the targeted allele was obtained in a total of 9 male chimeras from 5 independently-derived ES clones. Genomic Southern blots were hybridized at 42° C. as described above and washed in 0.2×SSC, 0.1% SDS at 42° C.

For whole leg analysis, legs of 14 week old mice were skinned, treated with 0.2 M EDTA in PBS at 4° C. for 4 weeks followed by 0.5 M sucrose in PBS at 4° C. For fiber number and size analysis, samples were directly mounted and frozen in isopentane as described (Brumback and Leech, Color Atlas of Muscle Histochemistry, pp. 9-33, PSG Publishing Company, Littleton, Mass., 1984). Ten to 30 μm sections were prepared using a cryostat and stained with hematoxylin and eosin. Muscle fiber numbers were determined from sections taken from the widest part of the tibialis cranialis muscle. Muscle fiber sizes were measured from photographs of sections of tibialis cranialis and gastrocnemius muscles. Fiber type analysis was carried out using the myosin ATPase assay after pretreatment at pH 4.35 as described (Cumming et al., Color Atlas of Muscle Pathology, pp. 184-185, 1994) and by immunohistochemistry using an antibody directed against type I myosin (MY32, Sigma) and the VECTASTAIN stating method (Vector Labs); in the immunohistochemical experiments, no staining was seen when the primary antibodies were left out. Carcasses were prepared from shaved mice by removing the all of the internal organs and associated fat and connective tissue. Fat content of carcasses from 4 month old males was determined as described (Leshner et al., Physiol. Behavior, 9:281, 1972).

For protein and DNA analysis, tissue was homogenized in 150 mM NaCl, 100 mM EDTA. Protein concentrations were determined using the BioRad protein assay. DNA was isolated by adding SDS to 1%, treating with 1 mg/ml proteinase K overnight at 55° C., extracting 3 times with phenol and twice with chloroform, and precipitating with ammonium acetate and EtOH. DNA was digested with 2 mg/ml RNase for 1 hour at 37° C., and following proteinase K digestion and phenol and chloroform extractions, the DNA was precipitated twice with ammonium acetate and EtOH.

Homologous targeting of the GDF-8 gene was seen in 13/131 gancyclovir/G418 doubly-resistant ES cell clones. Following injection of these targeted clones into blastocysts, we obtained chimeras from 5 independently-derived ES clones that produced heterozygous pups when crossed to C57BL/6 females (FIG. 12B). Genotypic analysis of 678 offspring derived from crosses of F1 heterozygotes showed 170+/+(25%), 380+/−(56%), and 128−/−(19%). Although the ratio of genotypes was close to the expected ratio of 1:2:1, the smaller than expected number of homozygous mutants appeared to be statistically significant (p<0.001).

Homozygous mutants were viable and fertile when crossed to C57BL/6 mice and to each other. Homozygous mutant animals, however, were approximately 30% larger than their heterozygous and wild type littermates (Table 1). The difference between mutant and wild type body weights appeared to be relatively constant irrespective of age and sex in adult animals. Adult mutants also displayed an abnormal body shape, with pronounced shoulders and hips. When the skin was removed from animals that had been sacrificed, it was apparent that the muscles of the mutants were much larger than those of wild type animals. The increase in skeletal muscle mass appeared to be widespread throughout the body. Individual muscles isolated from homozygous mutant animals weighed approximately 2-3 times more than those isolated from wild type littermates (Table 2). Although the magnitude of the weight increase appeared to roughly correlate with the level of GDF-8 expression in the muscles examined. To determine whether the increased muscle mass could account for the entire difference in total body weights between wild type and mutant animals or whether many tissues were generally larger in the mutants, we compared the total body weights to carcass weights. As shown in Table 3, the difference in carcass weights between wild type and mutant animals was comparable to the difference in total body weights. Moreover, because the fat content of mutant and wild type animals was similar, these data are consistent with all of the total body weight difference resulting from an increase in skeletal muscle mass, although we have not formally ruled out the possibility that differences in bone mass might also contribute to the differences in total body mass.

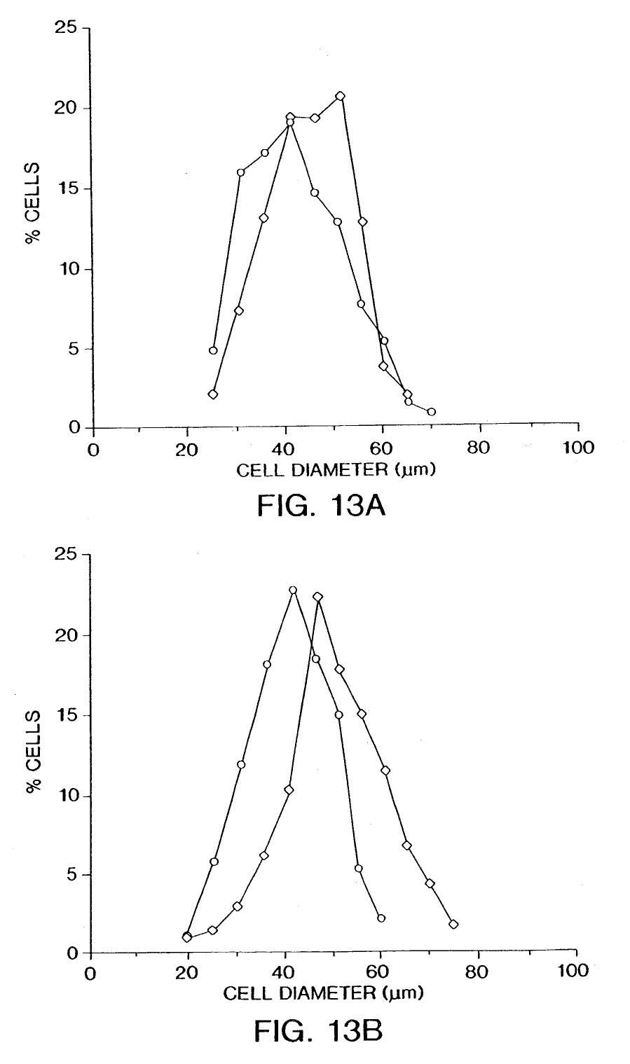

To determine whether the increase in skeletal muscle mass resulted from hyperplasia or from hypertrophy, histologic analysis of several different muscle groups was performed. The mutant muscle appeared grossly normal. No excess connective tissue or fat was seen nor were there any obvious signs of degeneration, such as widely varying fiber sizes (see below) or centrally-placed nuclei. Quantitation of the number of muscle fibers showed that at the widest portion of the tibialis cranialis muscle, the total cell number was 86% higher in mutant animals compared to wild type littermates {mutant=5470+/−121 (n=3), wild type=2936+/−288 (n=3); p<0.01}. Consistent with this result was the finding that the amount of DNA extracted from mutant muscle was roughly 50% higher than from wild type muscle {mutant=350 μg (n=4), wild type=233 μg (n=3) from pooled gastrocnemius, plantaris, triceps brachii, tibialis cranialis, and pectoralis muscles; p=0.05}. Hence, a large part of the increase in skeletal muscle mass resulted from muscle cell hyperplasia. However, muscle fiber hypertrophy also appeared to contribute to the overall increase in muscle mass. As shown in FIG. 13, the mean fiber diameter of the tibialis cranialis muscle and gastrocnemius muscle was 7% and 22% larger, respectively, in mutant animals compared to wild type littermates, suggesting that the cross-sectional area of the fibers was increased by approximately 14% and 49%, respectively. Notably, although the mean fiber diameter was larger in the mutants, the standard deviation in fiber sizes was similar between mutant and wild type muscle, consistent with the absence of muscle degeneration in mutant animals. The increase in fiber size was also consistent with the finding that the protein to DNA ratio (w/w) was slightly increased in mutant compared to wild type muscle {mutant=871+/−111 (n=4), wild type=624+/−85 (n=3); p<0.05}.

Table 4 shows a comparison between muscle weight (in grams) from wild-type (+/+), heterozygous (+/−) and a homozygous knock-out mice (−/−). The muscle mass is increased in heterozygous as compared to wild-type animals.

Finally, fiber type analysis of various muscles was carried out to determine whether the number of both type I (slow) and type II (fast) fibers was increased in the mutant animals. In most of the muscles examined, including the tibialis cranialis muscle, the vast majority of muscle fibers were type II in both mutant and wild type animals. Hence, based on the cell counts discussed above, the absolute number of type II fibers were increased in the tibialis cranialis muscle. In the soleus muscle, where the number of type I fibers was sufficiently high that we could attempt to quantitate the ratio of fiber types could be quantitated, the percent of type I fibers was decreased by approximately 33% in mutant compared to wild type muscle {wild type=39.2+/−8.1 (n=3), mutant=26.4+/−9.3 (n=4)}; however, the variability in this ratio for both wild type and mutant animals was too high to support any firm conclusions regarding the relative number of fiber types.

### EXAMPLE 9

Isolation of Rat and Chicken GDF-8

In order to isolate rat and chicken GDF-8 cDNA clones, skeletal muscle cDNA libraries prepared from these species were obtained from Stratagene and screened with a murine GDF-8 probe. Library screening was carried out as described previously (Lee, Mol. Endocrinol., 4:1034-1040) except that final washes were carried out in 2×SSC at 65° C. Partial sequence analysis of hybridizing clones revealed the presence of open reading frames highly related to murine and human GDF-8. Partial sequences of rat and chicken GDF-8 are shown in FIGS. 2C and 2D, respectively, and an alignment of the predicted rat (SEQ ID NO: 25) and chicken (SEQ ID NO: 23) GDF-8 amino acid sequences with those of murine (SEQ ID NO: 12) and human (SEQ ID NO: 14) GDF-8 are shown in FIG. 3B. Full length rat and chicken GDF-8 is shown in FIGS. 14D (SEQ ID NO: 25) and 14C (SEQ ID NO: 23), respectively and sequence alignment between murine, rat, human, baboon, porcine, ovine, bovine, chicken, and turkey sequences is shown in FIGS. 15A and 15B (SEQ ID NOS: 12, 25, 14, 19, 29, 31, 21, 23, and 27, respectively). All sequences contain an RSRR (SEQ ID NO: 51) sequence that is likely to represent the proteolytic processing site. Following this RSRR (SEQ ID NO: 51) sequence, the sequences contain a C-terminal region that is 100% conserved among all four species. The absolute conservation of the C-terminal region between species as evolutionarily far apart as humans and chickens, and baboons and turkeys, suggests that this region will be highly conserved in many other species as well.

Similar methodology was used to obtain the nucleotide and amino acid sequences for baboon (SEQ ID NO: 18 and 19, respectively; FIG. 14A); bovine (SEQ ID NO: 20 and 21, respectively; FIG. 14B); turkey (SEQ ID NO: 26 and 27, respectively; FIG. 14E); porcine (SEQ ID NO: 28 and 29, respectively; FIG. 14F); and ovine (SEQ ID NO: 30 and 31, respectively; FIG. 14G).

### EXAMPLE 10

GDF-11 Homology in Mammalian Species

The overall homology between GDF-11 and GDF-8 based upon their respective amino acid sequence is approximately 92% (see for example, PCT/US95/08543, which is incorporated herein by reference). Thus, it is expected that animals expressing GDF-8 and GDF-11 will display similar phenotypes. Similarly, animals having a disruption in a GDF-8 or GDF-11 gene will display similar phenotypes. The relationship of GDF-8 to GDF-11 will be further understood in light of the following examples, in which GDF-11 knockout mice were created.

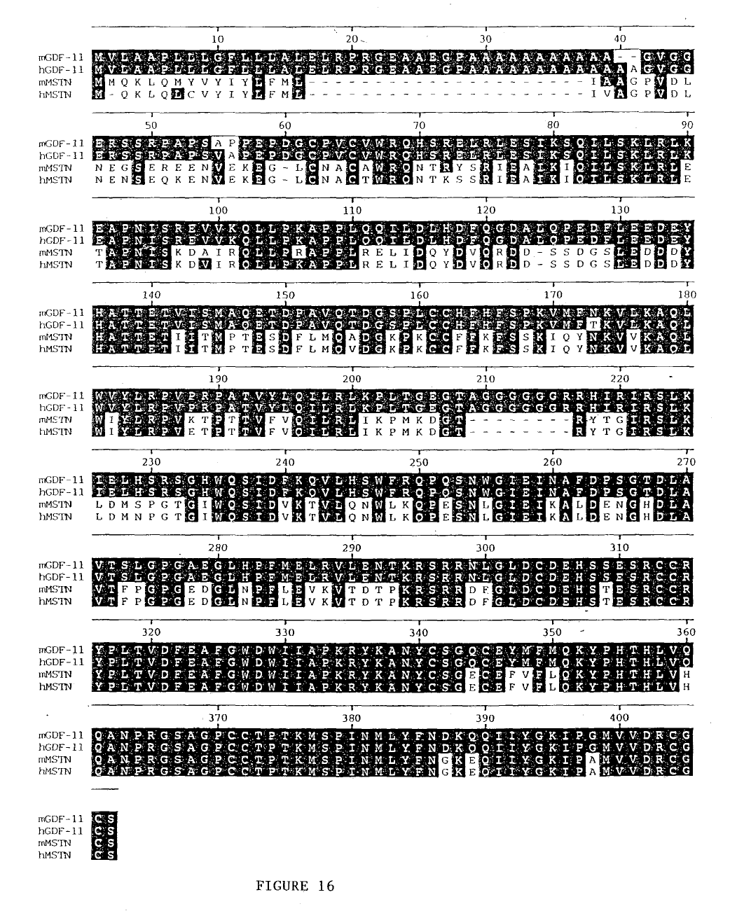

Like most other TGF-β family member, GDF-11 also appears to be highly conserved across species. By genomic Southern analysis, homologous sequences were detected in all mammalian species examined as well as in chickens and frogs (FIG. 16). In most species, the GDF-11 probe also detected a second, more faintly hybridizing fragment corresponding to the myostatin gene (McPherron et al., 1997).

### EXAMPLE 11

GDF-11 Knockout Mice

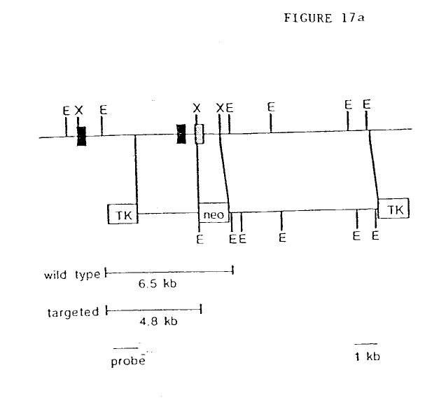

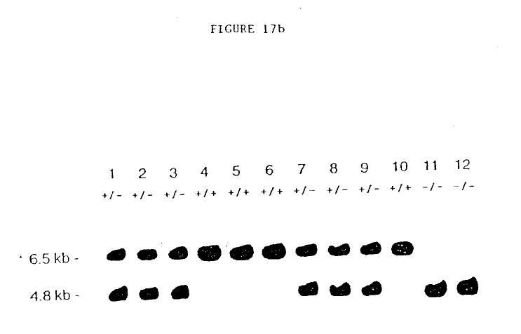

To determine the biological function of GDF-11, we disrupted the GDF-11 gene by homologous targeting in embryonic stem cells. A murine 129 SV/J genomic library was prepared in lambda FIXII vector according to the instructions provided by Stratagene. The structure of the GDF-11 gene was deduced from restriction mapping and partial sequencing of phage clones isolated from the library. Vectors for preparing the targeting construct were kindly provided by Philip Soriano and Kirk Thomas. To ensure that the resulting mice would be null for GDF-11 function, the entire mature C-terminal region was deleted and replaced by a neo cassette (FIGS. 17A and 17B). R1 ES cells were transfected with the targeting construct, selected with gancyclovir (2 μM) and G418 (250 μg/ml), and analyzed by Southern analysis. Homologous targeting of the GDF-11 gene was seen in 8/155 gancyclovir/G418 doubly resistant ES cell clones. Following injection of several targeted clones into C57BL/6J blastocysts, we obtained chimeras from one ES clone that produced heterozygous pups when crossed to both C57BL/6J and 129/SvJ females. Crosses of C57BL/6J/129/SvJ hybrid F1 heterozygotes produced 49 wild-type (34%), 94 heterozygous (66%) and no homozygous mutant adult offspring. Similarly, there were no adult homozygous null animals seen in the 129/SvJ background (32 wild-type (36%) and 56 heterozygous mutant (64%) animals).

To determine the age at which homozygous mutants were dying, we genotyped litters of embryos isolated at various gestational ages from heterozygous females that had been mated to heterozygous males. At all embryonic stages examined, homozygous mutant embryos were present at approximately the predicted frequency of 25%. Among hybrid newborn mice, the different genotypes were also represented at the expected Mendelian ratio of 1:2:1 (34+/+(28%), 61+/−(50%), and 28−/−(23%)). Homozygous mutant mice were born alive and were able to breath and nurse. All homozygous mutants died, however, within the first 24 hours after birth. The precise cause of death was unknown, but the lethality may have been related to the fact that the kidneys in homozygous mutants were either severely hypoplastic or completely absent. A summary of the kidney abnormalities in these mice is shown in FIG. 18.

### EXAMPLE 12

Anatomical Differences in GDF-11 Knockout Mice

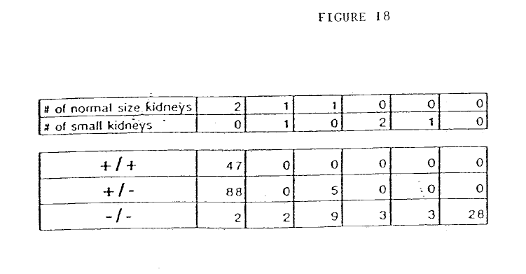

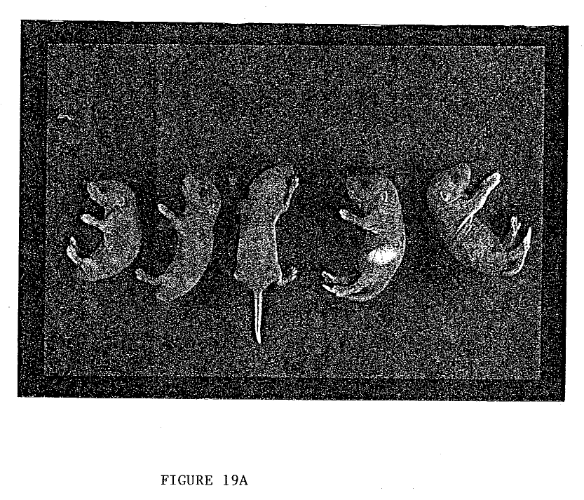

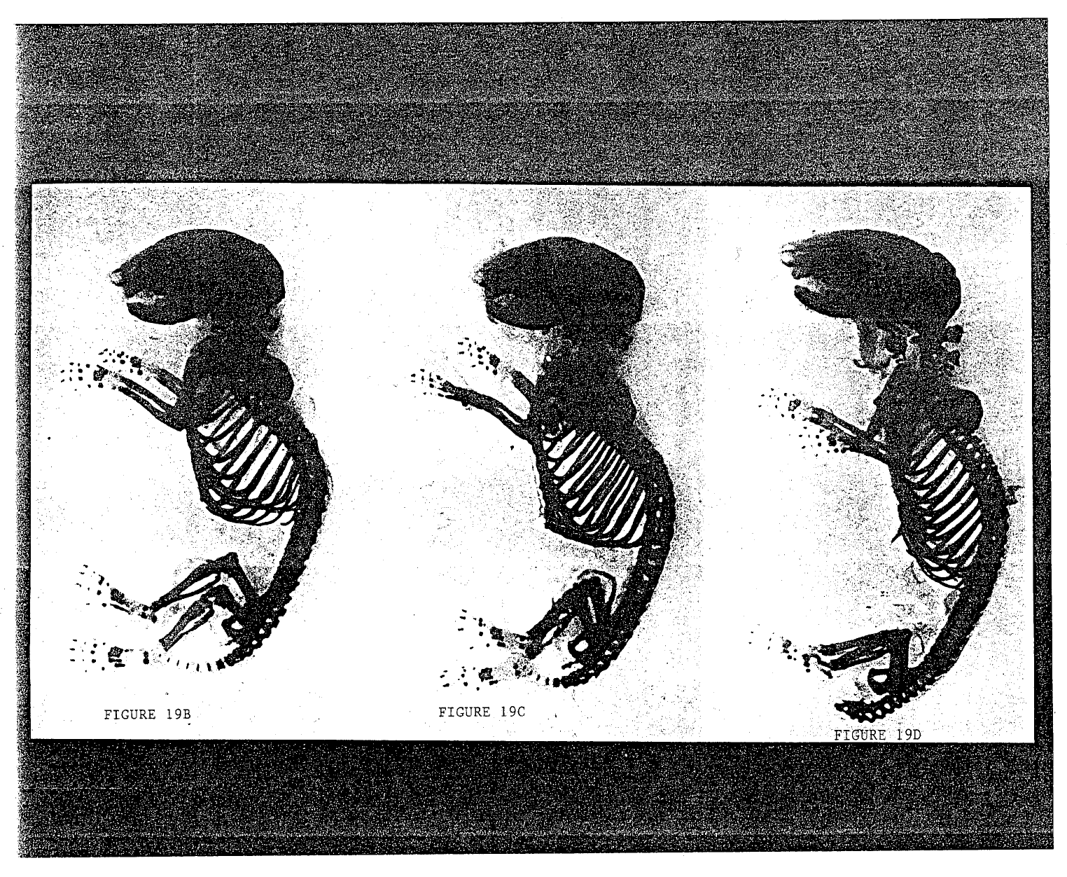

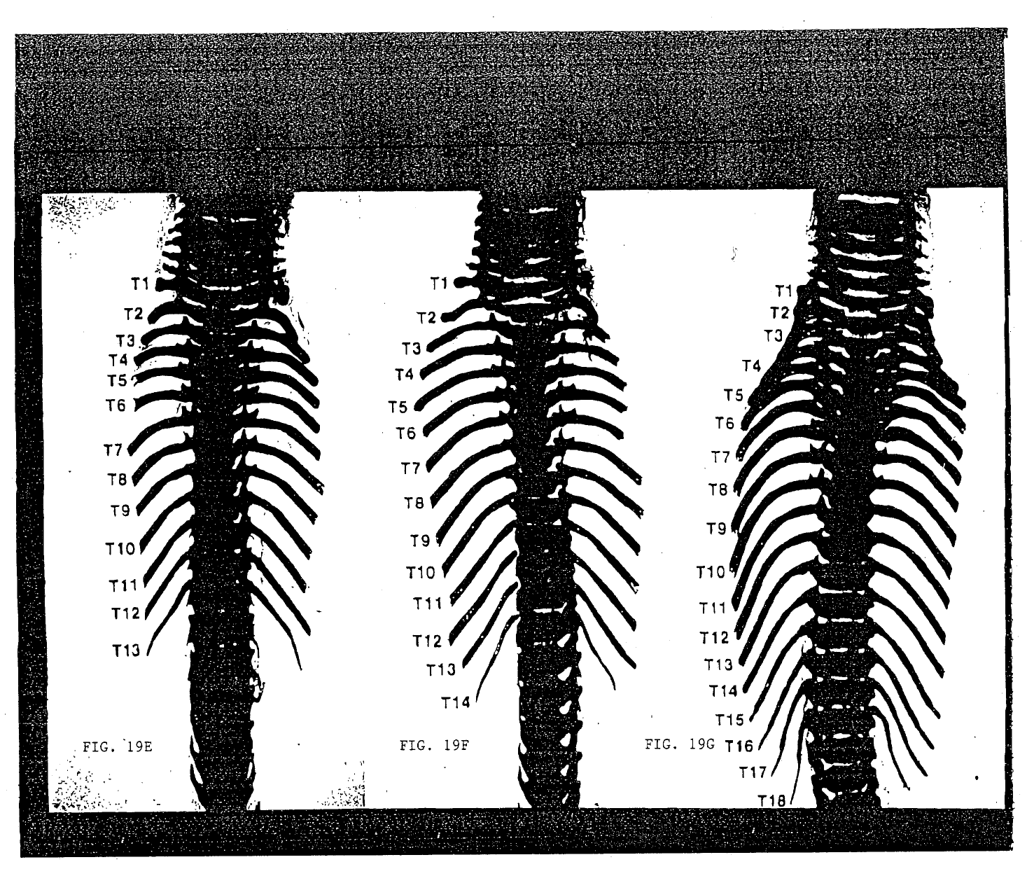

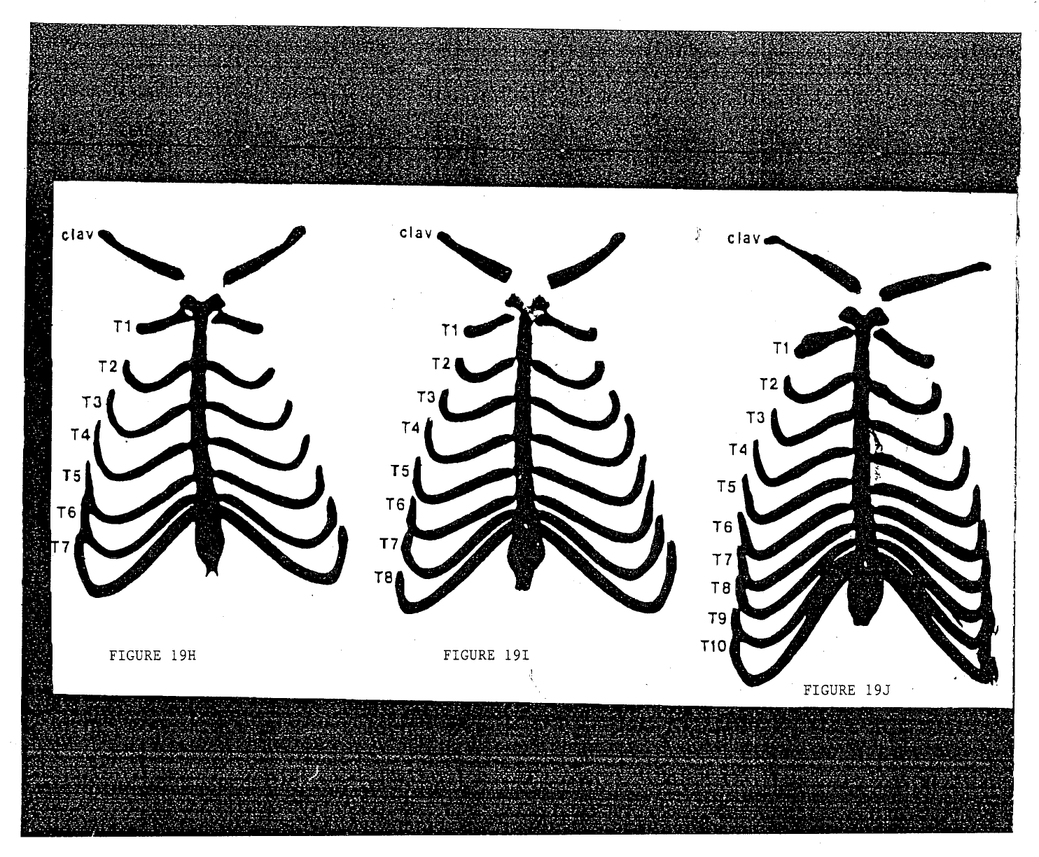

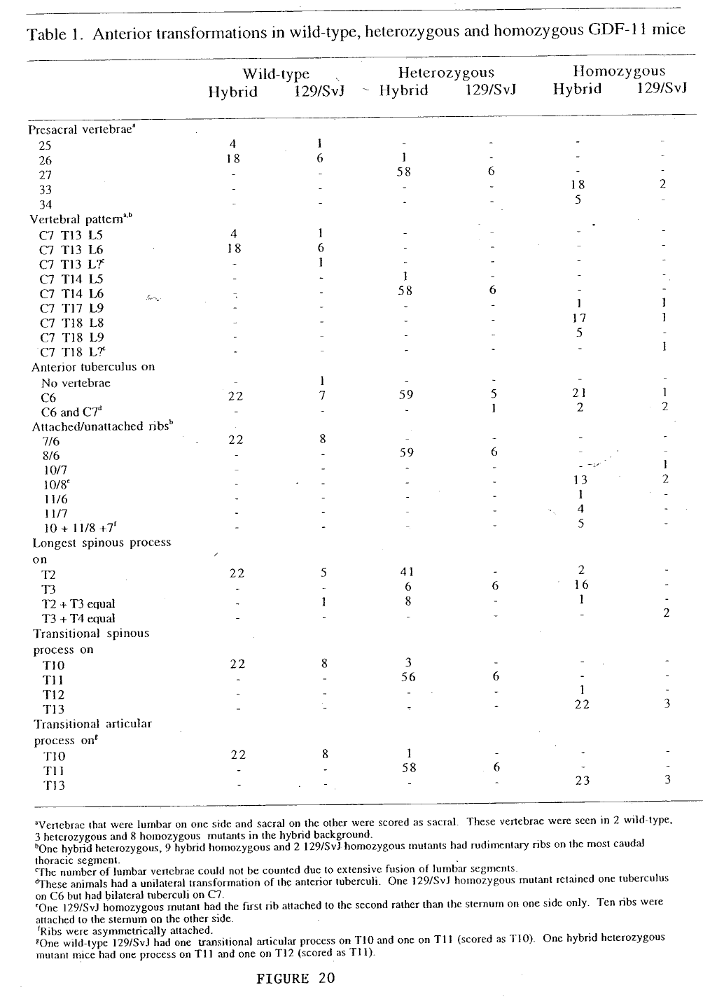

Homozygous mutant animals were easily recognizable by their severely shortened or absent tails (FIG. 19A). To further characterize the tail defects in these homozygous mutant animals, we examined their skeletons to determine the degree of disruption of the caudal vertebrae. A comparison of wild-type and mutant skeleton preparations of late stage embryos and newborn mice, however, revealed differences not only in the caudal region of the animals but in many other regions as well. In nearly every case where differences were noted, the abnormalities appeared to represent homeotic transformations of vertebral segments in which particular segments appeared to have a morphology typical of more anterior segments. These transformations, which are summarized in FIG. 20, were evident throughout the axial skeleton extending from the cervical region to the caudal region. Except for the defects seen in the axial skeleton, the rest of the skeleton, such as the cranium and limb bones, appeared normal.

Anterior transformations of the vertebrae in mutant newborn animals were most readily apparent in the thoracic region, where there was a dramatic increase in the number of thoracic (T) segments. All wild-type mice examined showed the typical pattern of 13 thoracic vertebrae each with its associated pair of ribs (FIGS. 19B and 19E). In contrast, homozygous mutant mice showed a striking increase in the number of thoracic vertebrae. All homozygous mutants examined had 4 to 5 extra pairs of ribs for a total of 17 to 18 (FIGS. 19D and 19G) although in over ⅓ of these animals, the 18th rib appeared to be rudimentary. Hence, segments that would normally correspond to lumbar (L) segments L1 to L4 or L5 appeared to have been transformed into thoracic segments in mutant animals.

Moreover, transformations within the thoracic region in which one thoracic vertebra had a morphology characteristic of another thoracic vertebra were also evident. For example, in wild-type mice, the first 7 pairs of ribs attach to the sternum, and the remaining 6 are unattached or free (FIGS. 19E and 19H). In homozygous mutants, there was an increase in the number of both attached and free pairs of ribs to 10-11 and 7-8, respectively (FIGS. 19G and 19J). Therefore, thoracic segments T8, T9, T10, and in some cases even T11, which all have free ribs in wild-type animals, were transformed in mutant animals to have a characteristic typical of more anterior thoracic segments, namely, the presence of ribs attached to the sternum. Consistent with this finding, the transitional spinous process and transitional articular processes which are normally found on T10 in wild-type animals were instead found on T13 in homozygous mutants (data not shown). Additional transformations within the thoracic region were also noted in certain mutant animals. For example, in wild-type mice, the ribs derived from T1 normally touch the top of the sternum. However, in 2/23 hybrid and ⅔ 129/SvJ homozygous mutant mice examined, T2 appeared to have been transformed to have a morphology resembling that of T1; that is, in these animals, the ribs derived from T2 extended to touch the top of the sternum. In these cases, the ribs derived from T1 appeared to fuse to the second pair of ribs. Finally, in 82% of homozygous mutants, the long spinous process normally present on T2 was shifted to the position of T3. In certain other homozygous mutants, asymmetric fusion of a pair of vertebrosternal ribs was seen at other thoracic levels.

The anterior transformations were not restricted to the thoracic region. The anterior most transformation that we observed was at the level of the 6th cervical vertebra (C6). In wild-type mice, C6 is readily identifiable by the presence of two anterior tuberculi on the ventral side. In several homozygous mutant mice, although one of these two anterior tuberculi was present on C6, the other was present at the position of C7 instead. Hence, in these mice, C7 appeared to have been partially transformed to have a morphology resembling that of C6. One other homozygous mutant had 2 anterior tuberculi on C7 but retained one on C6 for a complete C7 to C6 transformation but a partial C6 to C5 transformation.

Transformations of the axial skeleton also extended into the lumbar region. Whereas wild-type animals normally have only 6 lumbar vertebrae, homozygous mutants had 8-9. At least 6 of the lumbar vertebrae in the mutants must have derived from segments that would normally have given rise to sacral and caudal vertebrae as the data described above suggest that 4 to 5 lumbar segments were transformed into thoracic segments. Hence, homozygous mutant mice had a total of 33-34 presacral vertebrae compared to 26 presacral vertebrae normally present in wild-type mice. The most common presacral vertebral patterns were C7/T18/L8 and C7/T18/L9 for mutant mice compared to C7/T13/L6 for wild-type mice. The presence of additional presacral vertebrae in mutant animals was obvious even without detailed examination of the skeletons as the position of the hind limbs relative to the forelimbs was displaced posteriorly by 7-8 segments.

Although the sacral and caudal vertebrae were also affected in homozygous mutant mice, the exact nature of each transformation was not as readily identifiable. In wild-type mice, sacral segments S1 and S2 typically have broad transverse processes compared to S3 and S4. In the mutants, there did not appear to be an identifiable S1 or S2 vertebra. Instead, mutant animals had several vertebrae that appeared to have morphology similar to S3. In addition, the transverse processes of all 4 sacral vertebrae are normally fused to each other although in newborns often only fusions of the first 3 vertebrae are seen. In homozygous mutants, however, the transverse processes of the sacral vertebrae were usually unfused. In the caudal-most region, all mutant animals also had severely malformed vertebrae with extensive fusions of cartilage. Although the severity of the fusions made it difficult to count the total number of vertebrae in the caudal region, we were able to count up to 15 transverse processes in several animals. We were unable to determine whether these represented sacral or caudal vertebrae in the mutants because we could not establish morphologic criteria for distinguishing S4 from caudal vertebrae even in wild-type newborn animals. Regardless of their identities, the total number of vertebrae in this region was significantly reduced from the normal number of approximately 30. Hence, although the mutants had significantly more thoracic and lumber vertebrae than wild-type mice, the total number of segments was reduced in the mutants due to the truncation of the tails.

Heterozygous mice also showed abnormalities in the axial skeleton although the phenotype was much milder than in homozygous mice. The most obvious abnormality in heterozygous mice was the presence of an additional thoracic segment with an associated pair of ribs (FIGS. 19C and 19F). This transformation was present in every heterozygous animal examined, and in every case, the additional pair of ribs was attached to the sternum (FIG. 19). Hence, T8, whose associated rib normally does not touch the sternum, appeared to have been transformed to a morphology characteristic of a more anterior thoracic vertebra, and L1 appeared to have been transformed to a morphology characteristic of a posterior thoracic vertebra. Other abnormalities indicative of anterior transformations were also seen to varying degrees in heterozygous mice. These included a shift of the long spinous process characteristic of T2 by one segment to T3, a shift of the articular and spinous processes from T10 to T11, a shift of the anterior tuberculus on C6 to C7, and transformation of T2 to T1 where the rib associated with T2 touched the top of the sternum.

In order to understand the basis for the abnormalities in axial patterning seen in GDF-11 mutant mice, we examined mutant embryos isolated at various stages of development and compared them to wild-type embryos. By gross morphological examination, homozygous mutant embryos isolated up to day 9.5 of gestation were not readily distinguishable from corresponding wild-type embryos. In particular, the number of somites present at any given developmental age was identical between mutant and wild-type embryos, suggesting that the rate of somite formation was unaltered in the mutants. By day 10.5-11.5 p.c., mutant embryos could be easily distinguished from wild-type embryos by the posterior displacement of the hind limb by 7-8 somites. The abnormalities in tail development were also readily apparent at this stage. Taken together, these data suggest that the abnormalities observed in the mutant skeletons represented true transformations of segment identities rather than the insertion of additional segments, for example, by an enhanced rate of somitogenesis.

Alterations in expression of homeobox containing genes are known to cause transformations in Drosophila and in vertebrates. To see if the expression patterns of Hox genes (the vertebrate homeobox containing genes) were altered in GDF-11 null mutants we determined the expression pattern of 3 representative Hox genes, Hoxc-6, Hoxc-8 and Hoxc-11, in day 12.5 p.c. wild-type, heterozygous and homozygous mutant embryos by whole mount in situ hybridization. The expression pattern of Hoxc-6 in wild-type embryos spanned prevertebrae 8-15 which correspond to thoracic segments T1-T8. In homozygous mutants, however, the Hoxc-6 expression pattern was shifted posteriorly and expanded to prevertebrae 9-18 (T2-T11). A similar shift was seen with the Hoxc-8 probe. In wild-type embryos, Hoxc-8 was expressed in prevertebrae 13-18 (T6-T11) but, in homozygous mutant embryos, Hoxc-8 was expressed in prevertebrae 14-22 (T7-T15). Finally, Hoxc-11 expression was also shifted posteriorly in that the anterior boundary of expression changed from prevertebrae 28 tin wild-type embryos to prevertebrae 36 in mutant embryos. (Note that because the position of the hind limb is also shifted posteriorly in mutant embryos, the Hoxc-11 expression patterns in wild-type and mutant appeared similar relative to the hind limbs). These data provide further evidence that the skeletal abnormalities seen in mutant animals represent homeotic transformations.

The phenotype of GDF-11 mice suggested that GDF-11 acts early during embryogenesis as a global regulator of axial patterning. To begin to examine the mechanism by which GDF-11 exerts its effects, we determined the expression pattern of GDF-11 in early mouse embryos by whole mount in situ hybridization. At these stages the primary sites of GDF-11 expression correlated precisely with the known sites at which mesodermal cells are generated. Expression of GDF-11 was first detected at day 8.25-8.5 p.c. (8-10 somites) in the primitive streak region, which is the site at which ingressing cells form the mesoderm of the developing embryo. Expression was maintained in the primitive streak at day 8.75, but by day 9.5 p.c., when the tail bud replaces the primitive streak as the source of new mesodermal cells, expression of GDF-11 shifted to the tail bud. Hence at these early stages, GDF-11 appears to be synthesized in the region of the developing embryo where new mesodermal cells arise and presumably acquire their positional identity.

The phenotype of GDF-11 knockout mice in several respects resembles the phenotype of mice carrying a deletion of a receptor for some members of the TGF-β superfamily, the activin type IIB receptor (ActRIIB). As in the case of GDF-11 knockout mice, the ActRIB knockout mice have extra pairs of ribs and a spectrum of kidney defects ranging from hypoplastic kidneys to complete absence of kidneys. The similarity in the phenotypes of these mice raises the possibility that ActRIB may be a receptor for GDF-11. However, ActRIIB cannot be the sole receptor for GDF-11 because the phenotype of GDF-11 knockout mice is more severe than the phenotype of ActRIIB mice. For example, whereas the GDF-11 knockout animals have 4-5 extra pairs of ribs and show homeotic transformations throughout the axial skeleton, the ActRIIB knockout animals have only 3 extra pairs of ribs and do not show transformations at other axial levels. In addition, the data indicate that the kidney defects in the GDF-11 knockout mice are also more severe than those in ActRIIB knockout mice. The ActRIIB knockout mice show defects in left/right axis formation, such as lung isomerism and a range of heart defects that we have not yet observed in GDF-11 knockout mice. ActRIIB can bind the activins and certain BMPs, although none of the knockout mice generated for these ligands show defects in left/right axis formation.

If GDF-11 does act directly on mesodermal cells to establish positional identity, the data presented here would be consistent with either short range or morphogen models for GDF-11 action. That is, GDF-11 may act on mesodermal precursors to establish patterns of Hox gene expression as these cells are being generated at the site of GDF-11 expression, or alternatively, GDF-11 produced at the posterior end of the embryo may diffuse to form a morphogen gradient. Whatever the mechanism of action of GDF-11 may be, the fact that gross anterior/posterior patterning still does occur in GDF-11 knockout animals suggests that GDF-11 may not be the sole regulator of anterior/posterior specification. Nevertheless, it is clear that GDF-11 plays an important role as a global regulator of axial patterning and that further study of this molecule will lead to important new insights into how positional identity along the anterior/posterior axis is established in the vertebrate embryo.

Similar phenotypes are expected in GDF-8 knockout animals. For example, GDF-8 knockout animals are expected to have increased number of ribs, kidney defects and anatomical differences when compared to wild-type.

Although the invention has been described with reference to the presently preferred embodiment, it should be understood that various modifications can be made without departing from the spirit of the invention. Accordingly, the invention is limited only by the following claims.

What is claimed is:

1. A method of producing food products from a transgenic non-human animal having an increased number of ribs, comprising:
a) introducing a transgene disrupting or interfering with expression of growth differentiation factor-8 (GDF-8) into a pronuclear embryo or embryonic stem cells of the animal;
b) implanting the embryo or embryonic stem cells comprising the transgene into the oviduct of a pseudopregnant female of the same species, whereby the embryo or the embryonic stem cells can mature to full term progeny, thereby obtaining transgene positive progeny;
c) cross-breeding transgene-positive progeny with each other to obtain further transgene-positive progeny; and
d) processing the transgene-positive progeny having an increased number of ribs to obtain food products.

2. The method of claim 1, wherein the transgene comprises GDF-8 antisense polynucleotides.

3. The method of claim 1, wherein the transgene comprises a gene encoding a dominant negative GDF-8 polypeptide.

4. A method of producing food products from a transgenic avian species having an increased number of ribs, comprising:
a) introducing a transgene disrupting or interfering with expression of growth differentiation factor-8 (GDF-8) into a pronuclear embryo or embryonic stem cells of the avian species;
b) culturing the pronuclear embryo or embryonic stem cells under conditions whereby progeny are hatched;
c) cross-breeding transgene-positive progeny with each other; and
d) processing transgene-positive progeny having an increased number of ribs to obtain food products.

5. The method of claim 4, wherein the transgene comprises GDF-8 antisense polynucleotides.

6. The method of claim 4, wherein the transgene comprises a gene encoding a dominant negative GDF-8 polypeptide.

7. The transgenic animal of claim 4, wherein the transgene comprises a polynucleotide encoding a truncated GDF-8 polypeptide.

6. The method of claim 4, wherein the transgene comprises a gene encoding a dominant negative GDF-8 polypeptide.

7. The transgenic animal of claim 4, wherein the transgene comprises a polynucleotide encoding a truncated GDF-8 polypeptide.

8. A method of treating a chronic or acute renal disease in a subject having such a disease, comprising administering to the subject a reagent that affects growth differentiation factor-8 (GDF-8) activity or expression.

9. The method of claim 8, wherein the reagent is an agonist of GDF-8.

10. The method of claim 8, wherein the reagent is an antagonist of GDF-8.

11. The method of claim 10, wherein the antagonist is an antibody to GDF-8.

12. The method of claim 10, wherein the antagonist is an antisense polynucleotide to GDF-8.

13. A method of producing avian, porcine or bovine food products having an increased number of ribs, comprising
a) introducing a transgene disrupting or interfering with expression of growth differentiation factor-8 (GDF-8) into an embryo of an avian, porcine or bovine animal;
b) implanting the embryo into an oviduct of a pseudopregnant female, under conditions whereby progeny develop from the embryo;
c) cross-breeding transgene-positive progeny with each other; and
d) processing transgene-positive progeny having an increased number of ribs to obtain food products.

14. The method of claim 13, wherein the transgene comprises a gene encoding a dominant negative GDF-8 polypeptide.

15. A transgene-positive progeny animal produced by the method of claim 13, wherein the transgene comprises a polynucleotide encoding a truncated GDF-8 polypeptide.

16. A transgenic non-human animal having a transgene disrupting or interfering with expression of growth differentiation factor-8 (GDF-8) chromosomally integrated into the germ cells of the animal.

17. The transgenic animal of claim 16, wherein the animal is selected from the group of species consisting of avian, bovine, ovine, piscine, murine, and porcine.

18. The transgenic animal of claim 16, where the species is avian.

19. The transgenic animal of claim 16, where the species is bovine.

20. The transgenic animal of claim 16, where the species is porcine.

21. The transgenic animal of claim 16, where the species is ovine.

22. The transgenic animal of claim 16, where the species is piscine.

23. The transgenic animal of claim 16, wherein the transgene comprises a GDF-8 antisense polynucleotide.

24. The transgenic animal of claim 16, wherein the transgene comprises a polynucleotide encoding a dominant negative GDF-8 polypeptide.

25. The transgenic animal of claim 16, wherein the animal is homozygous or heterozygous for GDF-8 polynucleotide.

26. A chicken or turkey egg produced by the transgenic animal of claim 18.

27. Beef obtained from the transgenic animal of claim 19.

28. Milk obtained from the transgenic animal of claim 19.

29. Pork obtained from the transgenic animal of claim 20.

30. Lamb obtained from the transgenic animal of claim 21.

31. Chicken or turkey meat produced by the transgenic animal of claim 18.

32. A method of producing animal food products having increased muscle mass comprising:
a) introducing a transgene disrupting or interfering with expression of growth differentiation factor-8 (GDF-8) into a pronuclear embryo or embryonic stem cells of the animal;
b) implanting the embryo or embryonic stem cells into the oviduct of a pseudopregnant female of the same species, thereby allowing the embryo or embryonic stem cells to mature to full term progeny;
c) cross-breeding transgene-positive progeny with each other to obtain further transgene-positive progeny; and
d) processing the transgene-positive progeny to obtain food products.

33. The method of claim 32, wherein the transgene comprises GDF-8 antisense polynucleotides.

34. The method of claim 32, wherein the transgene comprises a polynucleotide encoding a dominant negative GDF-8 polypeptide.

35. A method of producing avian food products having reduced cholesterol levels, comprising:
a) introducing a transgene disrupting or interfering with expression of growth differentiation factor-8 (GDF-8) into an embryo of an avian animal;
b) culturing the embryo under conditions whereby progeny are hatched;
c) cross-breeding transgene-positive progeny with each other; and
d) processing the transgene-positive progeny to obtain food products.

36. The method of claim 35, wherein the transgene comprises GDF-8 antisense polynucleotides.

37. The method of claim 36, wherein the transgene comprises a polynucleotide encoding a dominant negative GDF-8 polypeptide.

38. The method of claim 36, wherein the transgene comprises a polynucleotide encoding a truncated GDF-8 polypeptide.

39. A method of inhibiting the growth regulating actions of GDF-8, comprising contacting a GDF-8 agent with fetal or adult muscle cells or progenitor cells.

40. The method of claim 39, wherein the agent is a monoclonal GDF-8 antibody, a GDF-8 antisense nucleic acid, or a GDF-8 dominant negative encoding nucleic acid sequence or polypeptide.

41. The method of claim 40, wherein the antibody is a humanized monoclonal antibody or a chimeric monoclonal antibody or fragment thereof.

42. The method of claim 39, wherein the agent is administered to a patient suffering from a disorder selected from a muscle wasting disease, neuromuscular disorder, muscle atrophy, or aging.

43. The method of claim 39, wherein the agent is administered to a patient suffering from a disorder selected from the group consisting of muscular dystrophy, spinal cord injury, traumatic injury, congestive obstructive pulmonary disease, acquired immunodeficiency syndrome, and cachexia.

44. The method of claim 39, wherein the agent is administered to a patient with muscle wasting disease or disorder by intravenous, intramuscular or subcutaneous injection.

45. The method of claim 40, wherein the monoclonal antibody is administered within a dose range between about 0.1 μg/kg to about 100 mg/kg.

46. The method of claim 40, wherein the monoclonal antibody is formulated in a formulation suitable for administration to a patient.

47. A method for treating a muscle or adipose tissue disorder in a subject, comprising administering a therapeutically effective amount of a GDF-8 agent to the subject, thereby inhibiting abnormal growth of muscle or adipose tissue.

48. The method as in claim 47, wherein the GDF-8 agent is a GDF-8 antisense polynucleotide, a polynucleotide encoding a dominant negative GDF-8 polypeptide, a GDF-8 antibody, or a polynucleotide encoding a truncated GDF-8 polypeptide.

49. The method of claim 47, wherein the disorder is a cancer selected from the group consisting of muscle, connective tissue, or bone.

50. The method of claim 47, wherein the subject has an obesity disorder.

51. A method of inhibiting the growth regulating actions of GDF-8 in a subject, comprising administering to the subject, a GDF-8 agent that inhibits the action of GDF-8 in the subject.

52. The method of 51, wherein the GDF-8 agent is a GDF-8 antisense polynucleotide, a polynucleotide encoding a dominant negative GDF-8 polypeptide, a GDF-8 antibody, or a polynucleotide encoding a truncated GDF-8 polypeptide.

53. A method for identifying a compound that affects GDF-8 activity or gene expression, comprising:
a) incubating the compound with GDF-8 polypeptide, or with a recombinant cell expressing GDF-8 under conditions sufficient to allow the components to interact; and
b) determining the effect of the compound on GDF-8 activity or expression.

54. The method of claim 53, wherein the effect is inhibition of GDF-8 activity or expression.

55. The method of claim 53, wherein the effect is stimulation of GDF-8 activity or expression.

56. An isolated polynucleotide encoding a truncated GDF-8 polypeptide, wherein the truncation is a loss of the C-terminal active fragment of GDF-8.

57. The isolated polynucleotide of claim 56, wherein the polynucleotide is set forth in FIG. 12A.
